(19) United States
(12) Patent Application Publication
(10) Pub. No.: US 2025/0339470 A1
(43) Pub. Date: Nov. 6, 2025

(54) METHODS AND COMPOSITIONS FOR MATURING CARDIOMYOCYTES

(71) Applicant: President and Fellows of Harvard College, Cambridge, MA (US)

(72) Inventors: Richard T. Lee, Cambridge, MA (US); Jia Liu, Cambridge, MA (US); Junya Aoyama, Cambridge, MA (US)

(21) Appl. No.: 19/098,940

(22) Filed: Apr. 2, 2025

Related U.S. Application Data
(60) Provisional application No. 63/573,395, filed on Apr. 2, 2024.

(51) Int. Cl. A61K 35/34; A61K 38/10; C07K 7/08; C12N 5/077

(52) U.S. Cl. CPC A61K 35/34; A61K 38/10; C07K 7/08; C12N 5/0657; C12N 2501/105; C12N 2501/115; C12N 2501/135; C12N 2501/165; C12N 2501/998; C12N 2506/45

(57) ABSTRACT

Disclosed herein are methods for induced pluripotent stem cell-derived cardiomyocytes (hiPSC-CMs) by combining the hiPSC-CMs with a self-assembling peptide (SAP). Also disclosed herein are compositions comprising induced pluripotent stem cell-derived cardiomyocytes and a self-assembling peptide.

### RELATED APPLICATIONS

This application claims the benefit of and priority to U.S. Provisional Application No. 63/573,395, filed on Apr. 2, 2024. The entire teachings of the above application are incorporated herein by reference.

### GOVERNMENT SUPPORT

This invention was made with government support under DK130673 awarded by National Institutes of Health (NIH). The government has certain rights in this invention.

### BACKGROUND OF THE INVENTION

Cardiac tissue engineering has emerged as a promising strategy for repairing damaged myocardium, particularly through the use of human induced pluripotent stem cell-derived cardiomyocytes (hiPSC-CMs) (1). However, despite significant advances, differentiated hiPSC-CMs generated by current methods often exhibit underdeveloped phenotypes characterized by fetal-like sarcomere organization, insufficient expression of adult myosin-heavy chains, and a propensity for arrhythmogenic automaticity (2, 3). hiPSC-CMs transplanted into cardiac tissue can exhibit increased automatic firing, or arrhythmogenic automaticity; this is thought to be the origin of potentially lethal ventricular arrhythmias observed in large animal models (4, 5). Establishing safe, efficacious hiPSC-CM transplantation technologies for human use remains an important translational goal.

### SEQUENCE LISTING

The Sequence Listing associated with this application is provided in .xml format in lieu of a paper copy and is hereby incorporated by reference into the specification. The name of the .xml file containing the Sequence Listing is HRVY-233-101.xml. The xml file is 16,984 bytes, was created on Jul. 2, 2025, and is being submitted electronically via Patent Center.

### SUMMARY OF THE INVENTION

The transplantation of stem cell-derived cardiomyocytes (e.g., human induced pluripotent stem cell-derived cardiomyocytes (hiPSC-CMs)) offers a promising treatment for heart failure, but arrhythmogenic automaticity arising from the transplanted cells can arise. Self-assembling peptides, such as RADA16, may accelerate the transition of hiPSC-CMs to adult-like gene expression profiles, enhanced sarcomere organization, and improved vascularization in the transplanted site. Flexible mesh nanoelectronics revealed fibrillation of transplanted hiPSC-CMs within the beating recipient heart, and RADA16 dramatically reduced the automaticity of hiPSC-CMs. Thus, there is a potential for self-assembling nanofibers to advance cardiac cell therapy and flexible nanomesh technology can improve safety.

Human induced pluripotent stem cell-derived cardiomyocytes (hiPSC-CMs) can exhibit increased automatic firing or arrhythmogenic automaticity when transplanted into cardiac tissue, which may lead to potentially lethal ventricular arrhythmias. Self-assembling peptides were utilized herein to provide injectable microenvironments for the hiPSC-CMs. The combination of a self-assembling peptide with hiPSC-CMs resulted in the cardiomyocytes exhibiting decreased arrhythmogenic automaticity, increased vascularization, and development of adult-like sarcomeres.

In some aspects, mature cardiomyocytes generated according to the methods described herein demonstrate many advantages; for example, they are electrically mature (e.g., exhibit decreased automaticity), contractility mature, and metabolically mature. In addition, the generated cardiomyocytes may provide a new platform for cell therapy (e.g., transplantation into a subject in need of additional and/or functional cardiomyocytes) and research.

Disclosed herein are methods of maturing a stem cell-derived cell or a precursor thereof. The methods may include contacting the stem cell-derived cell or precursor thereof with at least one self-assembling peptide.

In some embodiments, the stem cell-derived cell is selected from the group consisting of beta cells, alpha cells, delta cells, enterochromaffin cells, endothelial cells, satellite cells, cardiomyocytes, dermal cells, hematopoietic cells, and precursors thereof. In some embodiments, the self-assembling peptide is selected from the group consisting of RADA16, IEIK13, KLD12, and QLEL12, more specifically the self-assembling peptide is RADA16.

Also disclosed herein are methods of maturing a population of cardiomyocytes derived from human-induced pluripotent stem cells (hiPSC-CMs). The methods may include contacting the one or more hiPSC-CMs with a self-assembling peptide (SAP).

In some embodiments, the SAP is selected from the group consisting of RADA16, IEIK13, KLD12, and QLEL12. In one embodiment, the SAP comprises RADA16. In some embodiments, the population of hiPSC-CMs contacted with the SAP express one or more of MYH7/6, TNNI3/1, GJA1, MYL2 and KCNJ2. In some embodiments, the population of hiPSC-CMs contacted with the SAP exhibit increased expression of MYH7/6 as compared to control hiPSC-CMs. In some embodiments, the population of hiPSC-CMs contacted with the SAP exhibit increased expression of GJA1 as compared to control hiPSC-CMs. In some embodiments, the population of hiPSC-CMs contacted with the SAP exhibit increased expression of KCNJ2 as compared to control hiPSC-CMs. In some embodiments, the population of hiPSC-CMs contacted with the SAP exhibit increased expression of MYL2 as compared to control hiPSC-CMs. In some embodiments, the population of hiPSC-CMs contacted with the SAP exhibit decreased expression of HCN4 as compared to control hiPSC-CMs. In some embodiments, automaticity of the hiPSC-CMs is reduced after contact with the SAP.

In some embodiments, the population of hiPSC-CMs are contacted with the SAP upon co-administration of the population of hiPSC-CMs and the SAP to a subject. In some embodiments, the population of hiPSC-CMs are contacted with the SAP in a suspension prior to administration to a subject. In some embodiments, the subject is a mammal (e.g., a non-human mammal, such as a rat, or a human). In some embodiments, the population of hiPSC-CMs and the SAP are co-administered to the subject via syringe. Alternatively, in some embodiments, the population of hiPSC-CMs and the SAP are co-administered to the subject via catheter (e.g., a double lumen catheter).

In some embodiments, the SAP promotes engraftment and/or vascularization of the population of hiPSC-CMs in the subject. In some embodiments, vascularization is maintained in the subject for at least 1 month. In some embodiments, vascularization is maintained in the subject for at least 3 months. In some embodiments, the SAP promotes sarcomere organization of the population of hiPSC-CMs in the subject. In some embodiments, the population of hiPSC-CMs contacted with the SAP exhibited greater sarcomere organization upon administration to the subject, as compared to a population of hiPSC-CMs alone. In some embodiments, the population of hiPSC-CMs contacted with the SAP exhibited greater sarcomere organization upon administration to the subject for at least 3 months, as compared to a population of hiPSC-CMs alone. In some embodiments, the population of hiPSC-CMs contacted with the SAP exhibited increased sarcomere length upon administration to the subject, as compared to a population of hiPSC-CMs alone. In some embodiments, the SAP promotes improved electrophysiological integration, wherein the integration is monitored via mesh nanoelectronics (e.g., flexible mesh nanoelectronics).

Also disclosed herein are methods of treatment comprising administering to a subject in need thereof a composition comprising a population of cardiomyocytes derived from human-induced pluripotent stem cells (hiPSC-CMs) and a self-assembling peptide (SAP).

In some embodiments, the subject has, or is at risk of developing, a ventricular arrhythmia, decreased systolic heart function, chronic heart failure, congenital heart disease, or other heart disease. In some embodiments, the subject is a mammal (e.g., a non-human mammal, such as a rat, or a human).

In some embodiments, the population of hiPSC-CMs and the SAP are co-administered to the subject via syringe. In alternative embodiments, the population of hiPSC-CMs and the SAP are co-administered to the subject via catheter (e.g., a double lumen catheter). In some embodiments, the SAP is selected from the group consisting of RADA16, IEIK13, KLD12, and QLEL12, and more specifically comprises RADA16.

Further disclosed herein are uses of a composition in the manufacture of a medicament for treatment of a heart condition, wherein the treatment comprises administration of the medicament to a subject in need thereof, wherein the composition comprises a population of cardiomyocytes derived from human-induced pluripotent stem cells (hiPSC-CMs) and a self-assembling peptide (SAP).

In some embodiments, the subject has, or is at risk of developing, a ventricular arrhythmia, decreased systolic heart function, chronic heart failure, congenital heart disease, or other heart disease. In some embodiments, the subject is a mammal (e.g., a human or non-human mammal, such as a rat). In some embodiments, the population of hiPSC-CMs and the SAP are co-administered to the subject via syringe. In alternative embodiments, the population of hiPSC-CMs and the SAP are co-administered to the subject via catheter. In some embodiments, the SAP is selected from the group consisting of RADA16, IEIK13, KLD12, and QLEL12, and in one embodiment, the SAP comprises RADA16.

Also disclosed herein are pharmaceutical compositions comprising a population of cardiomyocytes derived from human-induced pluripotent stem cells (hiPSC-CMs) and a self-assembling peptide (SAP).

In some embodiments, the SAP is selected from the group consisting of RADA16, IEIK13, KLD12, and QLEL12. In one embodiment, the SAP comprises RADA16. In some embodiments, the composition further includes a pharmaceutically acceptable carrier or excipient. In some embodiments, the composition further includes RBI-PVbBB.

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual, 3rd cd., Cold Spring Harbor Laboratory Press, Cold Spring Harbor, 2001; Harlow, E. and Lane, D., Antibodies-A Laboratory Manual, Cold Spring Harbor Laboratory Press, Cold Spring Harbor, 1988; Freshney, R.I., “Culture of Animal Cells, A Manual of Basic Technique”, 5th ed., John Wiley & Sons, Hoboken, NJ, 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange; 10th cd. (2006) or 11th edition (July 2009). Non-limiting information regarding genes and genetic disorders is found in McKusick, V.A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, MD) and National Center for Biotechnology Information, National Library of Medicine (Bethesda, MD), as of May 1, 2010, World Wide Web URL: ncbi.nlm.nih.gov/omim/and in Online Mendelian Inheritance in Animals (OMIA), a database of genes, inherited disorders and traits in animal species (other than human and mouse), at omia.angis.org.au/contact.shtml. All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

### BRIEF DESCRIPTION OF THE DRAWINGS

The patent or application file contains at least one drawing executed in color. Copies of this patent or patent application publication with color drawings will be provided by the Office upon request and payment of the necessary fec.

FIGS. 1A-1H demonstrate engraftment and vascularization of injected human iPS-cardiomyocytes (hiPSC-CMs) in the rat left ventricular (LV) wall. Further, it was demonstrated that neovessels anastomosed with host vessels within the rat's left ventricular wall. FIG. 1A provides a schematic showing the variation of injection models: injection of hiPSC-CMs suspended in RBI-PVbBB (hiPSC-CM Alone), hiPSC-CMs with RADA16 suspended in RBI-PVbBB (hiPSC-CM+RADA16), and RADA16 suspended in RBI-PVbBB (RADA16 Alone). FIG. 1B provides images of samples stained with anti-human mitochondrial antibody (hMito: a marker of the injected hiPSC-CMs), anti-cardiac troponin T antibody (cTnT: a marker of troponin T which reacts to human and rat troponin T), and DAPI. FIG. 1C provides images of samples stained with cTnT, anti-platelet endothelial cell adhesion molecule-1 antibody (CD31: a marker of rat and human vascular endothelial cells), and DAPI. FIG. 1D provides a graph showing the area of the hiPSC-CMs (hMito positive area) in different rat samples (n=6) in each experimental group (hiPSC-CM Alone and hiPSC-CM+RADA16). FIG. 1E provides a graph showing the proportion of the vascularization area (CD31 positive) within the injected hiPSC-CMs (hMito positive) for different rat samples (n=6). Yellow arrows (FIG. 1B and FIG. 1D) show CD31 positive vessels negative for hMito. Student t-test. n=6/group. ns: not significant, **p<0.01. FIG. 1F provides a schematic showing the mechanism of GSL I infusion into the vessels. Infused GSL I solution flowed into rat blood vessels in the heart through the coronary arteries. Only after neovessels in the injection area anastomose with host vessels will these neovessels be stained with GSL I and thus detectable. FIG. 1G provides a graph showing the ratio of anastomosed vessel area (GSL I positive) within the transplanted hiPSC-CMs (hMito positive) in different rat samples (n=4). The area of anastomosed vessels was significantly greater in the hiPSC-CM+RADA16 group than in the hiPSC-CM Alone group. Student's t-test. n=4/group. *p<0.05. FIG. 1H shows a month after transplantation, vessels were stained with the GSL I infusion method, after which the cryosections of the heart were stained with anti-human mitochondria antibody (hMito), anti-platelet endothelial cell adhesion molecule-1 antibody (CD31), anti-cardiac troponin T antibody (cTnT), and DAPI. Many intact vessels were observed in the transplanted hiPSC-CMs area for each group.

FIGS. 2A-2C demonstrate RADA16 treatment accelerates sarcomeric development of hiPSC-CMs in the rat left ventricular wall. FIG. 2A shows relative expression of adult-like sarcomeric isoforms (MYH7 and MYL2) vs. fetal isoforms (MYH6 and MYL7) is greater at day 7 in hiPSC-CM+RADA16 relative to hiPSC-CM Alone, but this relative difference vanishes by day 21 where hiPSC-CM+RADA16 and hiPSC-CM Alone express similar ratios of adult to fetal sarcomeric isoforms. Red points represent cells expressing the fetal sarcomeric isoform (MYH6 or MYL7). Blue points represent cells expressing the adult sarcomeric isoform (MYH7 and MYL2). Orange points represent cells expressing both isoforms. FIGS. 2B-2C show differential gene expression of only hiPSC-CMs (as isolated based on human TTN expression) supports that RADA16 promotes more rapid sarcomeric development evidenced by differential expression of adult sarcomere isoforms MYH7 and MYL2 in (FIG. 2B) the comparison between hiPSC-CM+RADA16 and hiPSC-CM Alone at day 7 which does not persist to (FIG. 2C) the comparison at day 21. Differential gene expression was calculated using a Wilcoxon Ranked Sum test with Bonferroni multiple-testing correction with a p-adjusted threshold of α<0.05 and |mean log 2FC|>2. Enhanced Volcano was used for plotting.

FIGS. 3A-3H demonstrate sarcomere organization of hiPSC-CMs injected into the rats' left ventricle (LV) walls at 7 days and 3 months post-injection. Sarcomere organization was evaluated with the SarcOmere Texture Analysis (SOTA) method. FIG. 3A provides immunofluorescence images of hiPSC-CM Alone injection model stained with anti-cardiac troponin T antibody (cTnT), anti-human troponin I antibody (hTnI), and DAPI. The area positive for cTnT and hTnI exhibited unclear, fuzzy structures. FIG. 3B shows representative Haralick correlation curves from SOTA for hiPSC-CM Alone models. Sarcomere organization is the maximum amplitude of the decaying sinusoidal trace. FIG. 3C provides immunofluorescence images of hiPSC-CM+RADA16 injection model. hiPSC-CMs (positive for cTnT and hTnI) were observed in clearly segmented structures. FIG. 3D shows representative Haralick correlation curves from SOTA for hiPSC-CM+RADA16 models.

FIG. 3E shows comparison of sarcomere organization between hiPSC-CM+RADA16 and hiPSC-CM Alone at 7 days or 3 months post-injection, n=31, 34, 38, and 70 myofibril bundles for hiPSC-CM Alone and hiPSC-CM+RADA16 at 7 days, hiPSC-CM Alone and hiPSC-CM+RADA16 at 3 months, respectively, ****P<0.0001; one-way ANOVA with Games-Howell's post hoc test. FIG. 3F provides box plots of sarcomere lengths in each group; n=13, 30, 30, 50 sarcomeres, ***p<0.001; two-way ANOVA with Tukey's multiple comparison post hoc test. FIGS. 3G and 3H demonstrate vascularization of hiPSC-CMs injected into the rats' left ventricle (LV) walls at 3 months post-injection. Vascularization within the injected hiPSC-CMs area at 3 months post-injection was evaluated in cryosections stained with specific antibodies. FIG. 3G shows the injected hiPSC-CMs were positive for cTnT and hMito. Vessels within the injected hiPSC-CMs area were positive for CD31. Double positive area for cTnT and hMito represents injected hiPSC-CMs. CD31-positive vessels were observed in the injected hiPSC-CMs area, with more vessels in the hiPSC-CM+RADA16 group than in the hiPSC-CM Alone group. FIG. 3H provides a graph quantifying the ratio of CD31 positive area within the injected hiPSC-CMs area for each group. The ratio of the vessel area was significantly higher in the hiPSC-CM+RADA16 group than in the hiPSC-CM Alone group at the 3-month time point. Student's t-test. n=4/group. **p<0.01.

FIGS. 4A-4G demonstrate electrical signal recording using flexible mesh nanoelectronics on cardiac tissue with hiPSC-CM Alone and hiPSC-CM+RADA16 injection at 80 days of post-transplantation. FIG. 4A provides a schematic illustration of the electrical signal recording workflow. FIG. 4B shows representative bright-field (BF) microscopic images of flexible mesh nanoelectronics; (i) overview of the flexible mesh nanoelectronics on the wafer; (ii) free-standing flexible mesh nanoelectronics after releasing from the wafer.

FIG. 4C provides optical images of electrical signal recording setup; (i) overview of the electrical signal recording setup; (ii) representative BF microscopic images of the flexible mesh nanoelectronics on the reference site of the heart; (iii) representative BF microscopic images of the flexible mesh nanoelectronics on injection site of the heart. FIG. 4D provides representative raw voltage traces showing electrical activity recorded from reference and hiPSC-CM Alone injection sites. FIG. 4E provides representative raw voltage traces showing electrical activity recorded from reference and hiPSC-CM+RADA16 injection sites. FIG. 4F shows zoomed-in view of representative raw voltage traces with activation time (blue) and peak time (red) detection denoted by markers showing electrical activity in the hiPSC-CM injection site. FIG. 4G provides heatmaps illustrating the calculated activation delay and detected peak amplitudes of three occurrences of automaticity rhythm electrical signals from the electrical mapping, projected onto the recording electrode arrangement.

FIGS. 5A-5G demonstrate cardiac automaticity and sinus rhythms across RADA16 treatment conditions. FIG. 5A provides a schematic showing the experiment design. FIG. 5B provides a bar plot illustrating the trend of percentage of samples with automaticity rhythm over time. FIG. 5C provides raster plots denoting the detected sinus rhythm (black) and automaticity rhythm (red) over three second recordings in (i) hiPSC-CM Alone injection sites, (ii) hiPSC-CM+RADA16 injection sites, (iii) RADA16 injection sites, and (iv) SHAM (saline) injection sites. (n=5 three-second samples per subject, n=3 subjects per treatment condition). FIG. 5D provides a density plot showing the distribution of normalized ISI of automaticity rhythm (red) and sinus rhythm (blue). (n=3 subjects per treatment condition).

FIG. 5E provides Violin and scatter plots showing the distribution of normalized ISI of all detected spikes. (n=3 subjects per treatment condition) ****p<0.0001, **p<0.01, two-tailed, unpaired t-test. FIG. 5F provides Violin and scatter plots showing the distribution of ratios of automaticity rhythm spike counts over sinus rhythm spike counts detected within three seconds of recording. (n=5 three-second samples per subject, n=3 subjects per treatment condition) ****p<0.0001, **p<0.01, two-tailed, unpaired t-test. FIG. 5G shows Power spectrum density (PSD) plots of representative raw voltage traces from reference sites (blue) and each injection site (red). (n=2 channels per subject).

FIGS. 6A-6F demonstrate RADA16 uniquely promotes the adult-like gene expression of hiPSC-CMs compared to other self-assembling peptides (SAPs). The study investigates the gene expression patterns of hiPSC-CMs cultured for seven days on various SAP-coated substrates, focusing on their progression toward an adult-like cardiomyocyte phenotype. The findings are summarized as follows: FIG. 6A shows the relative mRNA expression levels of myosin heavy chain isoforms (MYH6 and MYH7) indicate a shift from fetal to adult-like myofibril expression. RADA16 induced the most pronounced increase in the MYH7/MYH6 expression ratio compared to other SAPs. FIG. 6B shows changes in troponin isoform expression, represented by the TNNI1 to TNNI3 transition, were also evaluated. RADA16 promoted a higher TNNI3/TNNI1 expression ratio, consistent with adult human myofibril characteristics, outperforming other SAPs. FIG. 6C shows RADA16 supported the highest expression of GJA1, a gene encoding gap junction proteins, suggesting enhanced intercellular connectivity. FIG. 6D shows mRNA expression of HCN4, associated with pacemaker activity, decreased across all SAPs relative to the control, indicating reduced pacemaker phenotype. FIG. 6E shows the potassium channel gene KCNJ2 exhibited its highest expression in the RADA16 condition. FIG. 6F shows NANOG expression, a marker of pluripotency, significantly declined on RADA16 compared to other SAPs. Control: Geltrex. Statistical analysis: one-way ANOVA with post hoc Tukey test. n=3 per group; *p<0.05, **p<0.01.

FIGS. 7A-7D demonstrate injected hiPSC-CMs are retained in the injection site at day 7. FIG. 7A shows the retention of injected hiPSC-CMs was evaluated via an in vivo imaging system (IVIS). Images show the retention of hiPSC-CMs in the left ventricular (LV) wall (ex vivo) injected with versus without RADA16. The labeled hiPSC-CMs were injected, followed by harvest on day 7. The retention of the labeled hiPSC-CMs in the rats' LV wall was then evaluated with the IVIS. FIG. 7B provides a graph showing the maximum radiant efficiency 7 days after cell injection in 6 different models. Yellow arrows point to the area of injected labeled hiPSC-CMs. Student T-test. n=6/group. ns: not significant. FIG. 7C provides images showing the retention of hiPSC-CMs in the LV wall (in vivo) with versus without RADA16 evaluated with the IVIS. The labeled hiPSC-CMs were injected, followed by an evaluation of cell retention by IVIS on day 7. FIG. 7D provides a graph showing the maximum radiant efficiency 7 days after cell injection in 6 different models of each group. Yellow arrows point to the area of injected labeled hiPSC-CMs. Student T-test. n=6/group. ns: not significant.

FIGS. 8A-8B demonstrate hiPSC-CMs have distinct transcriptional patterns relative to the surrounding myocardium as identifiable by human Titin (TTN) expression. Differential gene expression between UMAP clusters enables the identification of distinct clusters of hiPSC-CMs at (FIG. 8A): 7 days post-injection and (FIG. 8B) 21 days post-injection based on markers for human cardiomyocytes such as TTN, MYH6, NPPA, and MYH7, which have distinctive expression patterns relative to the surrounding rat myocardium.

FIG. 9 demonstrates slide-seq plots of human cardiac troponin showing evidence of spatial clustering of transplanted hiPSC-CMs. Spatial expression plots of human cardiac troponin (GRCh38 TNNT2: blue) indicate successful engraftment of hiPSC-CMs by 7 days, with more significant engraftment occurring by 21 days irrespective of RADA16 treatment.

FIGS. 10A-10C demonstrate ion channel gene expression is relatively uniform relative to TTN expression. The expression of (FIG. 10A) HCN4, (FIG. 10B) RYR2, and (FIG. 10C) ATP2A2 remains consistent relative to TTN expression irrespective of treatment and timepoint. (Points in red are TTN-expressing beads. Points in blue are ion channel-expressing beads. Points in orange are beads expressing both TTN and the channel marker of interest).

FIGS. 11A-11H demonstrate schematic illustration and electrical characterization of the fabrication process for flexible mesh nanoelectronics. FIG. 11A shows a clean glass wafer. FIG. 11B shows deposition of the Ni sacrificial layer on a glass wafer. FIG. 11C shows patterning of the bottom SU-8 passivation layer. FIG. 11D shows deposition and patterning of Cr/Au/Cr interconnect. FIG. 11E shows deposition and patterning of Pt electrodes. FIG. 11F shows patterning of the top SU-8 passivation layer. FIG. 11G shows electrochemical impedance spectroscopy and phase response across frequencies for mesh electronics with Pt black. FIG. 11H shows statistics of impedance measurements at 1 KHz across different samples.

FIGS. 12A-12B demonstrate electrical activity and signal propagation delays at the injection site. FIG. 12A provides representative raw voltage traces showing electrical activity recorded from the injection site with detected sinus rhythm (red). FIG. 12B provides heatmaps illustrating the smoothed detected activation propagation delay of automaticity rhythm (blue) from the electrical mapping, projected onto the recording electrode arrangement.

FIGS. 13A-13C demonstrate electrical activity normalized peak amplitudes and trends of automaticity rhythm. FIG. 13A provides representative raw voltage traces showing electrical activity recorded from the injection site with detected sinus rhythm (red). FIG. 13B provides heatmaps illustrating the detected and normalized peak amplitudes of automaticity rhythm from the electrical mapping, projected onto the recording electrode arrangement. FIG. 13C provides a line plot showing the trend of detected peak amplitudes of automaticity rhythm.

FIGS. 14A-14B provide a comparison of electrical activity at reference and injection sites for hiPSC-CM Alone and hiPSC-CM+RADA16 at 15 days. FIG. 14A provides representative raw voltage traces showing electrical activity recorded from reference (blue) and injection site (red) with detected sinus rhythm (gray) and automaticity rhythm (purple) over one second for hiPSC-CM Alone at 15 days. FIG. 14B provides representative raw voltage traces showing electrical activity recorded from reference (blue) and injection site (red) with detected sinus rhythm (gray) and automaticity rhythm (purple) over one second for hiPSC-CM+RADA16 at 15 days.

FIGS. 15A-15B provide a comparison of electrical activity at reference and injection sites for hiPSC-CM Alone and hiPSC-CM+RADA16 at 30 days. FIG. 15A provides representative raw voltage traces showing electrical activity recorded from reference (blue) and injection site (red) with detected sinus rhythm (gray) and automaticity rhythm (purple) over one second for hiPSC-CM Alone at 30 days. FIG. 15B provides representative raw voltage traces showing electrical activity recorded from reference (blue) and injection site (red) with detected sinus rhythm (gray) and automaticity rhythm (purple) over one second for hiPSC-CM+RADA16 at 30 days.

FIGS. 16A-16B provide a comparison of electrical activity at reference and injection sites for hiPSC-CM Alone and hiPSC-CM+RADA16 at 80 days. FIG. 16A provides representative raw voltage traces showing electrical activity recorded from reference (blue) and injection site (red) with detected sinus rhythm (gray) and automaticity rhythm (purple) over one second for hiPSC-CM Alone at 80 days. FIG. 16B provides representative raw voltage traces showing electrical activity recorded from reference (blue) and injection site (red) with detected sinus rhythm (gray) and automaticity rhythm (purple) over one second for hiPSC-CM+RADA16 at 80 days.

FIGS. 17A-17B provide a comparison of electrical activity at reference and injection sites for hiPSC-CM Alone and hiPSC-CM+RADA16 at 135 days. FIG. 17A provides representative raw voltage traces showing electrical activity recorded from reference (blue) and injection site (red) with detected sinus rhythm (gray) and automaticity rhythm (purple) over one second for hiPSC-CM Alone at 135 days. FIG. 17B provides representative raw voltage traces showing electrical activity recorded from reference (blue) and injection site (red) with detected sinus rhythm (gray) and automaticity rhythm (purple) over one second for hiPSC-CM+RADA16 at 135 days.

FIGS. 18A-18B demonstrates electrical activity at reference and injection sites for RADA16 and SHAM at 30 days. FIG. 18A provides representative raw voltage traces showing electrical activity recorded from reference (blue) and injection site (red) with detected sinus rhythm (gray) over one second for RADA16 at 30 days. FIG. 18B provides representative raw voltage traces showing electrical activity recorded from reference (blue) and injection site (red) with detected sinus rhythm (gray) over one second for SHAM at 30 days.

FIG. 19 provides a schematic illustration of inter-spike intervals (ISIs). Representative raw voltage traces with sinus spikes (blue) and automaticity spikes (red) highlighted.

FIGS. 20A-20C demonstrate normalized inter-spike-interval (ISI) analyses for 15 days past treatment. FIG. 20A provides heatmaps illustrating the activity distribution of normalized sinus rhythm ISI of 3 representative samples. FIG. 20B provides heatmaps illustrating the activity distribution of normalized automaticity rhythm ISI of 3 representative samples. FIG. 20C provides heatmaps illustrating the activity distribution of normalized sinus (blue) and automaticity (red) rhythm ISI of 3 representative samples.

FIGS. 21A-21C demonstrate normalized inter-spike-interval (ISI) analyses for 30 days past treatment. FIG. 21A provides heatmaps illustrating the activity distribution of normalized sinus rhythm ISI of 3 representative samples. FIG. 21B provides heatmaps illustrating the activity distribution of normalized automaticity rhythm ISI of 3 representative samples. FIG. 21C provides heatmaps illustrating the activity distribution of normalized sinus (blue) and automaticity (red) rhythm ISI of 3 representative samples.

FIGS. 22A-22C demonstrate normalized inter-spike interval (ISI) analyses for 80 days past treatment. FIG. 22A provides heatmaps illustrating the activity distribution of normalized sinus rhythm ISI of 3 representative samples. FIG. 22B provides heatmaps illustrating the activity distribution of normalized automaticity rhythm ISI of 3 representative samples. FIG. 22C provides heatmaps illustrating the activity distribution of normalized sinus (blue) and automaticity (red) rhythm ISI of 3 representative samples.

FIGS. 23A-23C demonstrate normalized inter-spike-interval (ISI) analyses for 135 days past treatment. FIG. 23A provides heatmaps illustrating the activity distribution of normalized sinus rhythm ISI of 3 representative samples. FIG. 23B provides heatmaps illustrating the activity distribution of normalized automaticity rhythm ISI of 3 representative samples. FIG. 23C provides heatmaps illustrating the activity distribution of normalized sinus (blue) and automaticity (red) rhythm ISI of 3 representative samples.

FIGS. 24A-24C demonstrate normalized inter-spike-interval (ISI) analyses for 30 days past treatment. FIG. 24A provides heatmaps illustrating the activity distribution of normalized sinus rhythm ISI of 3 representative samples. FIG. 24B provides heatmaps illustrating the activity distribution of normalized automaticity rhythm ISI of 3 representative samples. FIG. 24C provides heatmaps illustrating the activity distribution of normalized sinus (blue) and automaticity (red) rhythm ISI of 3 representative samples.

FIGS. 25A-25C provide a normalized inter-spike-interval (ISI) summary. FIG. 25A provides heatmaps illustrating the activity distribution of normalized sinus rhythm ISI of different treatments. FIG. 25B provides heatmaps illustrating the activity distribution of normalized automaticity rhythm ISI of different treatments. FIG. 25C provides heatmaps illustrating the activity distribution of normalized sinus (blue) and automaticity (red) rhythm ISI of different treatments.

FIG. 26 shows a mammalian response to injury. Cardiac regeneration occurs in the embryo and neonate but not in an adult mammal. See Uygur and Lee, Dev Cell, 2016.

FIG. 27 demonstrates a goal of a research study—to deliver mature, hypoimmunogenic cardiomyocytes capable of engraftment to injured myocardium to treat heart failure.

FIG. 28 shows nuclear fallout data from Karolinska indicated that new heart cells are made at a rate of around 1%/year, and even less as you age. See Bergmann et al., Science 2009.

FIG. 29 shows multi-isotope Imaging Mass Spectrometry (MIMS) is a high resolution (˜30 nm) approach to measuring isotopes precisely. See Lechene C, J Biol. 2006.

FIG. 30 shows MIMS scanning reveals cardiomyocytes unambiguously.

FIG. 31 shows MIMS quantitates DNA synthesis in the mouse heart after 8 week labeling. See Senyo Nature 2013.

FIG. 32 demonstrate that exercise causes increased cardiomyocyte size in some beneficial manner. See Hill and Olson, NEJM 2008. Mice often run voluntarily and this makes new heart cells.

FIG. 33 shows voluntary running is a natural stress-free form of exercise for mice.

FIG. 34 shows voluntary running increases new heart cell generation in the young heart. See Vujic and Lerchenmüller, Nature Comm, 2018.

FIG. 35 shows voluntary running restores cardiomyocyte birth in the old heart. See Lerchenmüller and Vujic et al, Circulation 2022.

FIG. 36 shows the rate of cardiomyogenesis increased with exercise by 4.6-fold in young heart and 6.5-fold in aged heart. See Lerchenmüller and Vujic et al, Circulation 2022.

FIG. 37 suggests that driving cardiomyocyte division for a long period will likely be unsafe.

FIG. 38 considers whether the inventors could use human cardiomyocytes for chronic systolic cardiomyopathy. Human iPS cells from skin fibroblasts were shown to differentiate to cardiomyocytes in 10 days with chemically defined, xeno-free conditions. Large quantities of human cardiomyocytes may be made using 3D culture

FIG. 39 suggests human cardiomyocytes derived from ES cells can be used at acute myocardial infarction. Chuck Murry's lab at the University of Washington has shown feasibility of embryonic stem cell-derived cardiomyocytes in non-human primates. Transient non-sustained ventricular tachycardia is a problem. This strategy requires immunosuppression. See Chong et al. Nature 510, 273-77, 2014.

FIG. 40 demonstrates delivery of immature stem cell-derived cardiomyocytes to large animal models leads to transient ventricular tachycardia. Immature stem cell-derived cardiomyocytes exhibit automaticity. Inadequate maturation of stem cell-derived cardiomyocytes may be a major barrier to clinical translation. See Chong et al. Nature 2014; Liu et al. Nat Biotechnol 2018; Romagnuolo et al. Stem Cell Rep 2019.

FIG. 41 demonstrates gene editing could get past arrhythmia barrier. “Gene editing to prevent ventricular arrhythmias associated with cardiomyocyte cell therapy.” Marchiano S et al. Cell Stem Cell 2023.

FIG. 42 shows injectable, prevascularized myocardium. See Hsieh et al. Annu Rev Physiol 2006; 68:51-66. Co-delivery of iPSC-derived endothelial cells with iPSC-derived cardiomyocytes may enhance maturation and improve engraftment.

FIG. 43 shows co-culture of ECs with cardiac progenitor cells improves maturation of cardiomyocytes. See Dunn et al. (Palacek) Biotechnol J 2019; 14:e1800725.

FIG. 44 shows self-assembling peptides are short peptide sequences that self-assemble in physiological conditions to form hydrogels. They can be injected in catheters and are in clinical use for hemostasis during surgery.

FIGS. 45A-45C demonstrate self-assembling peptides (SAP) promote vascularization and engraftment in the ventricular wall. FIG. 45A show self-assembling peptides (SAP) provide nanofiber micro environments: alignment of cells, vascular networks, scaffold>>CM maturation. FIG. 45B shows capillary-like structures within the microenvironment (RAD16-II) in the LV wall of mice. ECs: isolectin-FITC. FIG. 45C shows vessels within the peptide contain RBC. PECAM-1 (CD31). See Circulation 2005 Feb. 1; 111 (4): 442-50.

FIG. 46 demonstrates injectable self-assembling peptide nanofibers can create vascularized homes for human stem-cell cardiomyocytes.

FIG. 47 shows human cardiomyocyte maturation improved by RADA16 in vitro. See Circ Res 2020 Apr. 10; 126 (8): 1086-1106.

FIG. 48 shows cardiomyocyte maturation improved in vivo.

FIG. 49 shows cardiomyocyte maturation accelerated by unbiased gene expression analysis. Maturation marker genes expressed earlier in the CM+RADA16 group in rat ventricle.

FIG. 50 demonstrates vascularization at 1-month (Anastomoses).

FIG. 51 shows vascularization increased by RADA16 at 3-months. CD31 positive vessels were observed in the area of injected hiPS-CMs significantly more in the RADA16 co-injection group than in the CM alone group.

FIG. 52 demonstrates stretchable mesh nanoelectronics. See Li et al. Nano Lett 2019; 19:5781-5789.

FIG. 53 shows EC mediated CM maturation revealed by electronics and scRNA-seq. See Z. Lin, et al. Science Advances, 2023.

FIGS. 54A-54H shows electrophysiological nanomesh signals from human cardiomyocytes in rat hearts. FIGS. 54A-54D provide raw trace recordings using two 32-electrode arrays placed on the atrium part (Group A) and implanted cardiomyocytes (Group B) region of the rat heart. FIGS. 54E-54F provide spike sorting from representative channels showing both heart rhythm with (FIG. 54E)/without (FIG. 54F) arrhythmia signals from implanted CMs. FIG. 54G shows Langendorff setup of ex vivo heart recording. FIG. 54H shows a section of the ventricle and implanted human CMs.

FIG. 55 demonstrates ventricular fibrillation of human CMs inside of the rat myocardium. Nanomesh electrophysiological signals are shown for human cardiomyocytes 30 days after injection into a rat and for human cardiomyocytes 30 days after injection into a rat in combination with RADA16.

FIG. 56 demonstrates that 3 months after transplantation into the athymic rat heart, some hiPS-derived cardiomyocytes appear to be in ventricular bigeminy. This was not observed when hiPS-derived cardiomyocytes are injected with RADA16.

FIG. 57 shows human cardiomyocyte automaticity in the rat LV myocardium. The atria was removed, the recording was taken at human CM injection, 30 days without RADA16 nanofibers.

FIG. 58 demonstrates various approaches to heart regeneration. See Garbern and Lee, Developmental Cell 2022.

FIG. 59 provides a schematic for overcoming barriers to clinical translation.

### DETAILED DESCRIPTION OF THE INVENTION

In some instances, stem cell derived cells produced in vitro may have difficulties reaching fully maturity in vitro. The stem cell derived cells may be administered in vivo such that the maturation of the stem cell derived cells may occur in vivo. In some instances the maturation of the stem cell derived cell administered in vivo matures over an extended period of time. However, it has been shown herein that the co-administration of a stem cell derived cell with a self-assembling peptide results in the accelerated maturation of the stem cell derived cell such that it reaches a mature state faster than if it was administered alone.

Aspects of the disclosure relate to compositions, methods, kits, and agents for maturing stem cell-derived cells (e.g., pluripotent stem cell-derived cells, such as human pluripotent stem cell-derived cells) by contacting the stem cell-derived cells with a self-assembling peptide (SAP), and mature stem cell-derived cells produced by those methods, kits, and agents for use in cell therapies, assays, and various methods of treatment.

### Definitions

For convenience, certain terms employed herein, in the specification, examples and appended claims are collected here. Unless otherwise defined, all technical and scientific terms used herein have the same meaning as commonly understood by one of ordinary skill in the art to which this invention belongs.

As used herein, the term “adult cell” refers to a cell found throughout the body after embryonic development.

The term “progenitor” or “precursor” cell are used interchangeably herein and refer to cells that have a cellular phenotype that is more primitive (i.e., is at an earlier step along a developmental pathway or progression than is a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate.

The term “phenotype” refers to one or a number of total biological characteristics that define the cell or organism under a particular set of environmental conditions and factors, regardless of the actual genotype.

The term “pluripotent” as used herein refers to a cell with the capacity to differentiate to more than one differentiated cell type, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. It should be noted that simply culturing such cells does not, on its own, render them pluripotent. Reprogrammed pluripotent cells (e.g., iPS cells as that term is defined herein) also have the characteristic of the capacity of extended passaging without loss of growth potential, relative to primary cell parents, which generally have capacity for only a limited number of divisions in culture.

As used herein, the terms “iPS cell” and “induced pluripotent stem cell” are used interchangeably and refer to a pluripotent stem cell artificially derived (e.g., induced or by complete reversal) from a non-pluripotent cell, typically an adult somatic cell, for example, by inducing a forced expression of one or more genes.

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retrodifferentiation” by persons of ordinary skill in the art. As used herein, the term “pluripotent stem cell” includes embryonic stem cells, induced pluripotent stem cells, placental stem cells, etc.

The term “stem cell-derived cell” or “differentiated cell” is meant any primary cell that is not, in its native form, pluripotent as that term is defined herein. Stated another way, the term “differentiated cell” or “stem cell-derived cell” refers to a cell of a more specialized cell type derived from a cell of a less specialized cell type (e.g., a stem cell such as an induced pluripotent stem cell) in a cellular differentiation process. Non-limiting examples of stem cell-derived cells include alpha cells, beta cells, delta cells, enterochromaffin cells, satellite cells, cardiomyocytes, hematopoietic cells, dermal cells, endothelial cells, etc., and the precursors thereof.

The terms “endogenous cardiomyocyte” or “endogenous mature cardiomyocyte” are used herein to refer to a mature cardiomyocyte. A mature cardiomyocyte may exhibit electrical maturity, contractile maturity, and/or metabolic maturity. The phenotype of a cardiomyocyte is well known by persons of ordinary skill in the art, and includes, for example, ability to spontaneously beat, expression of markers such as cardiac troponin, TNNT2, TNNI3, myosin heavy chain, MYH6, MYH7, ryanodine receptor (RyR), sodium channel protein SCN5a, potassium voltage-gated channel KCNJ2, ATP2A2, PPARGCla, Cx43, as well as distinct morphological characteristics such as organized sarcomeres, having rod shaped cells, and having T-tubules.

As used herein “non-naturally occurring cardiomyocyte,” “non-native cardiomyocyte,” and “mature cardiomyocyte,” all refer to cardiomyocytes produced by the methods as disclosed herein, e.g., cardiomyocytes matured from induced pluripotent stem cell-derived cardiomyocytes. The cardiomyocytes may be ventricular-, atrial-, and/or nodal-type cardiomyocytes, or a mixed population of cardiomyocytes. Cardiomyocytes may exhibit one or more features which may be shared with endogenous cardiomyocytes, including, but not limited to, capacity to beat spontaneously, are electrically mature, metabolically mature, contractility mature, exhibit appropriate expression of one or more gene markers (e.g., MYH7, MYH6, TNNI3, TNNI1, GJA1/CX43, MYL2 and KCNJ2), exhibit appropriate expression of one or more quiescence markers, exhibit appropriate morphological characteristics (e.g., rod shaped cells and organized sarcomeres), and expandability in culture. However non-naturally occurring cardiomyocytes are not identical to and are distinguishable from endogenous cardiomyocytes as described herein, including distinction on the basis of gene expression. For example, non-naturally occurring cardiomyocytes may express similar proteins but at distinguishable expression levels as compared to endogenous cardiomyocytes.

The term “cardiomyocyte marker” refers to, without limitation, proteins, peptides, nucleic acids, polymorphism of proteins and nucleic acids, splice variants, fragments of proteins or nucleic acids, elements, and other analytes which are specifically expressed or present in endogenous cardiomyocytes. Exemplary cardiomyocyte markers include, but are not limited to, cardiac troponin T (TNNT2), cardiac troponin I (TNNI3), potassium channel KCNJ2, repressor element-1 silencing transcription actor (REST), ryanodine receptor (RyR), sodium channel (SCN5a), myosin regulatory light chain 2 (MYL2) and those described in Yang et al. Circ. Res. 2014; 114 (3): 511-23.

The term “immature cardiomyocyte” as used herein is meant a cardiomyocyte that is immature (e.g., electrical, metabolic, and/or contractile). Immature cardiomyocytes display automaticity or pacemaker-like activity, have a higher resting membrane potential and slower upstroke velocity, low expression of skeletal troponin I, have a less organized sarcomere structure, lower maximum contractile force, do not have T-tubules, predominantly acquire energy through glycolysis (rather than oxidative phosphorylation), and may be a senescent state rather than a quiescent state.

As used herein, the term “proliferation” means growth and division of cells. In some embodiments, the term “proliferation” as used herein in reference to cells refers to a group of cells that can increase in number over a period of time.

In the context of cell ontogeny, the adjective “differentiated,” or “differentiating” is a relative term meaning a “differentiated cell” is a cell that has progressed further down the developmental pathway than the cell it is being compared with. Thus, stem cells can differentiate to lineage-restricted precursor cells (such as a mesodermal stem cell), which in turn can differentiate into other types of precursor cells further down the pathway (such as a cardiomyocyte precursors), and then to an end-stage differentiated cell, which plays a characteristic role in a certain tissue type, and may or may not retain the capacity to proliferate further.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaccous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecules having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

As used herein, the term “contacting” (e.g., contacting at least one immature cardiomyocyte or a precursor thereof with a maturation factor, or combination of maturation factors) is intended to include incubating the differentiation medium and/or agent and the cell together in vitro (e.g., adding the maturation factors to cells in culture). In some embodiments, the term “contacting” is not intended to include the in vivo exposure of cells to the compounds as disclosed herein that may occur naturally in a subject (e.g., exposure that may occur as a result of a natural physiological process). In some embodiments, the term “contacting” is intended to include co-culturing at least one immature cardiomyocyte with at least one secondary cell (e.g., at least one endothelial cell). The step of contacting at least one immature cardiomyocyte or a precursor thereof with a maturation factor as in the embodiments described herein can be conducted in any suitable manner. For example, the cells may be treated in three-dimensional culture. In some embodiments, the cells are treated in conditions that promote the formation of cardiomyocytes. The disclosure contemplates any conditions which promote the formation of mature cardiomyocytes. Examples of conditions that promote the formation of mature cardiomyocytes include, without limitation, suspension culture in low attachment tissue culture plates, spinner flasks, and aggrewell plates. In some embodiments, the inventors have observed that mature cardiomyocytes have remained stable in media. In some aspects, serum (e.g., heat inactivated fetal bovine serum) is added prior to dissociating and re-plating the cells.

It is understood that the cells contacted with a maturation factor (e.g., a cardiomyocyte maturation factor) can also be simultaneously or subsequently contacted with another agent, such as other differentiation agents or environments to stabilize the cells, or to differentiate or mature the cells further.

Similarly, at least one immature cardiomyocyte or a precursor thereof can be contacted with at least one cardiomyocyte maturation factor and then contacted with at least another cardiomyocyte maturation factor. In some embodiments, the cell is contacted with at least one cardiomyocyte maturation factor, and the contact is temporally separated, and in some embodiments, a cell is contacted with at least one cardiomyocyte maturation factor substantially simultaneously. In some embodiments, the cell is contacted with at least two, at least three, at least four, at least five, at least six, at least seven, at least eight, at least nine, or at least 10 cardiomyocyte maturation factors

The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art.

The term “cell line” refers to a population of largely or substantially identical cells that has typically been derived from a single ancestor cell or from a defined and/or substantially identical population of ancestor cells. The cell line may have been or may be capable of being maintained in culture for an extended period (e.g., months, years, for an unlimited period of time). It may have undergone a spontaneous or induced process of transformation conferring an unlimited culture lifespan on the cells. Cell lines include all those cell lines recognized in the art as such. It will be appreciated that cells acquire mutations and possibly epigenetic changes over time such that at least some properties of individual cells of a cell line may differ with respect to each other. In some embodiments, a cell line comprises a cardiomyocyte described herein.

The term “exogenous” refers to a substance present in a cell or organism other than its native source. For example, the terms “exogenous nucleic acid” or “exogenous protein” refer to a nucleic acid or protein that has been introduced by a process involving the hand of man into a biological system such as a cell or organism in which it is not normally found or in which it is found in lower amounts. A substance will be considered exogenous if it is introduced into a cell or an ancestor of the cell that inherits the substance. In contrast, the term “endogenous” refers to a substance that is native to the biological system.

The terms “genetically modified” or “engineered” cell as used herein refers to a cell into which an exogenous nucleic acid has been introduced by a process involving the hand of man (or a descendant of such a cell that has inherited at least a portion of the nucleic acid). The nucleic acid may for example contain a sequence that is exogenous to the cell, it may contain native sequences (i.e., sequences naturally found in the cells) but in a non-naturally occurring arrangement (e.g., a coding region linked to a promoter from a different gene), or altered versions of native sequences, etc. The process of transferring the nucleic acid into the cell can be achieved by any suitable technique. Suitable techniques include calcium phosphate or lipid-mediated transfection, electroporation, and transduction or infection using a viral vector. In some embodiments the polynucleotide or a portion thereof is integrated into the genome of the cell. The nucleic acid may have subsequently been removed or excised from the genome, provided that such removal or excision results in a detectable alteration in the cell relative to an unmodified but otherwise equivalent cell. It should be appreciated that the term genetically modified is intended to include the introduction of a modified RNA directly into a cell (e.g., a synthetic, modified RNA). Such synthetic modified RNAs include modifications to prevent rapid degradation by endo- and exo-nucleases and to avoid or reduce the cell's innate immune or interferon response to the RNA. Modifications include, but are not limited to, for example, (a) end modifications, e.g., 5′ end modifications (phosphorylation dephosphorylation, conjugation, inverted linkages, etc.), 3′ end modifications (conjugation, DNA nucleotides, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, as well as (d) internucleoside linkage modifications, including modification or replacement of the phosphodiester linkages. To the extent that such modifications interfere with translation (i.e., results in a reduction of 50% or more in translation relative to the lack of the modification—e.g., in a rabbit reticulocyte in vitro translation assay), the modification is not suitable for the methods and compositions described herein.

The term “expression” refers to the cellular processes involved in producing RNA and proteins and as appropriate, secreting proteins, including where applicable, but not limited to, for example, transcription, translation, folding, modification and processing. “Expression products” include RNA transcribed from a gene and polypeptides obtained by translation of mRNA transcribed from a gene.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated”.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched from.

The term “substantially pure,” with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified,” with regard to a population of cardiomyocytes, refers to a population of cells that contain fewer than about 20%, more preferably fewer than about 15%, 10%, 8%, 7%, most preferably fewer than about 5%, 4%, 3%, 2%, 1%, or less than 1%, of cells that are not cardiomyocytes as defined by the terms herein. In some embodiments, the present invention encompasses methods to expand a population of cardiomyocytes, wherein the expanded population of cardiomyocytes is a substantially pure population of cardiomyocytes.

The terms “enriching” or “enriched” are used interchangeably herein and mean that the yield (fraction) of cells of one type is increased by at least 10% over the fraction of cells of that type in the starting culture or preparation.

The terms “renewal” or “self-renewal” or “proliferation” are used interchangeably herein, are used to refer to the ability of stem cells to renew themselves by dividing into the same non-specialized cell type over long periods, and/or many months to years. In some instances, proliferation refers to the expansion of cells by the repeated division of single cells into two identical daughter cells.

The term “modulate” is used consistently with its use in the art, i.e., meaning to cause or facilitate a qualitative or quantitative change, alteration, or modification in a process, pathway, or phenomenon of interest. Without limitation, such change may be an increase, decrease, or change in relative strength or activity of different components or branches of the process, pathway, or phenomenon. A “modulator” is an agent that causes or facilitates a qualitative or quantitative change, alteration, or modification in a process, pathway, or phenomenon of interest.

A “marker” as used herein is used to describe the characteristics and/or phenotype of a cell. Markers can be used for selection of cells comprising characteristics of interests. Markers will vary with specific cells. Markers are characteristics, whether morphological, functional or biochemical (enzymatic) characteristics of the cell of a particular cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate or dedifferentiate along particular lineages. Markers may be detected by any method available to one of skill in the art. Markers can also be the absence of a morphological characteristic or absence of proteins, lipids etc. Markers can be a combination of a panel of unique characteristics of the presence and absence of polypeptides and other morphological characteristics.

The term “selectable marker” refers to a gene, RNA, or protein that when expressed, confers upon cells a selectable phenotype, such as resistance to a cytotoxic or cytostatic agent (e.g., antibiotic resistance), nutritional prototrophy, or expression of a particular protein that can be used as a basis to distinguish cells that express the protein from cells that do not. Proteins whose expression can be readily detected such as a fluorescent or luminescent protein or an enzyme that acts on a substrate to produce a colored, fluorescent, or luminescent substance (“detectable markers”) constitute a subset of selectable markers. The presence of a selectable marker linked to expression control elements native to a gene that is normally expressed selectively or exclusively in pluripotent cells makes it possible to identify and select somatic cells that have been reprogrammed to a pluripotent state. A variety of selectable marker genes can be used, such as neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene. Detectable markers include green fluorescent protein (GFP) blue, sapphire, yellow, red, orange, and cyan fluorescent proteins and variants of any of these. Luminescent proteins such as luciferase (e.g., firefly or Renilla luciferase) are also of use. As will be evident to one of skill in the art, the term “selectable marker” as used herein can refer to a gene or to an expression product of the gene, e.g., an encoded protein.

In some embodiments the selectable marker confers a proliferation and/or survival advantage on cells that express it relative to cells that do not express it or that express it at significantly lower levels. Such proliferation and/or survival advantage typically occurs when the cells are maintained under certain conditions, i.e., “selective conditions.” To ensure an effective selection, a population of cells can be maintained under conditions and for a sufficient period of time such that cells that do not express the marker do not proliferate and/or do not survive and are eliminated from the population or their number is reduced to only a very small fraction of the population. The process of selecting cells that express a marker that confers a proliferation and/or survival advantage by maintaining a population of cells under selective conditions so as to largely or completely eliminate cells that do not express the marker is referred to herein as “positive selection,” and the marker is said to be “useful for positive selection.” Negative selection and markers useful for negative selection are also of interest in certain of the methods described herein. Expression of such markers confers a proliferation and/or survival disadvantage on cells that express the marker relative to cells that do not express the marker or express it at significantly lower levels (or, considered another way, cells that do not express the marker have a proliferation and/or survival advantage relative to cells that express the marker). Cells that express the marker can therefore be largely or completely eliminated from a population of cells when maintained in selective conditions for a sufficient period of time.

The terms “subject” and “individual” are used interchangeably herein, and refer to an animal, for example, a human from whom cells can be obtained and/or to whom treatment, including prophylactic treatment, with the cells as described herein, is provided. For treatment of those infections, conditions or disease states which are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like.

The terms “treat,” “treating,” “treatment,” etc., as applied to an isolated cell, include subjecting the cell to any kind of process or condition or performing any kind of manipulation or procedure on the cell. As applied to a subject, the terms “treat,” “treating,” “treatment,” etc. refer to providing medical or surgical attention, care, or management to an individual. The individual is usually ill or injured, or at increased risk of becoming ill relative to an average member of the population and in need of such attention, care, or management. It may include administering to a subject an effective amount of a composition so that the subject exhibits a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. The term “treatment” includes prophylaxis. Those in need of treatment include those already diagnosed with a condition (e.g., cardiac disorder or disease), as well as those likely to develop a condition due to genetic susceptibility or other factors.

The term “tissue” refers to a group or layer of specialized cells which together perform certain special functions. The term “tissue-specific” refers to a source of cells from a specific tissue.

The terms “decrease,” “reduced,” “reduction,” “decrease” or “inhibit” are all used herein generally to mean a decrease by a statistically significant amount. However, for avoidance of doubt, “reduced,” “reduction,” “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90%, or up to and including a 100% decrease (i.e., absent level as compared to a reference sample), or any decrease between 10-100% as compared to a reference level.

The terms “increased,” “increase,” “enhance” or “activate” are all used herein to generally mean an increase by a statically significant amount; for the avoidance of any doubt, the terms “increased,” “increase,” “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90%, or up to and including a 100% increase or any increase between 10-100% as compared to a reference level, or at least about a 2-fold, or at least about a 3-fold, or at least about a 4-fold, or at least about a 5-fold, or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus, for example, references to “the method” includes one or more methods, and/or steps of the type described herein and/or which will become apparent to those persons skilled in the art upon reading this disclosure and so forth.

### Stem Cells

Stem cells are cells that retain the ability to renew themselves through mitotic cell division and can differentiate into a diverse range of specialized cell types. The two broad types of mammalian stem cells are: embryonic stem (ES) cells that are found in blastocysts, and adult stem cells that are found in adult tissues. In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues. Pluripotent stem cells can differentiate into cells derived from any of the three germ layers.

While certain embodiments are described below in reference to the use of stem cells for producing cardiomyocytes (e.g., mature cardiomyocytes) or precursors thereof, germ cells may be used in place of, or with, the stem cells to provide at least one cardiomyocyte, using similar protocols as the illustrative protocols described herein. Suitable germ cells can be prepared, for example, from primordial germ cells present in human fetal material taken about 8-11 weeks after the last menstrual period. Illustrative germ cell preparation methods are described, for example, in Shamblott et al., Proc. Natl. Acad. Sci. USA 95:13726, 1998 and U.S. Pat. No. 6,090,622.

ES cells, e.g., human embryonic stem cells (hESCs) or mouse embryonic stem cells (mESCs), with a virtually endless replication capacity and the potential to differentiate into most cell types, present, in principle, an unlimited starting material to generate the differentiated cells for clinical therapy (stemcells.nih.gov/info/scireport/2006report.htm, 2006). One possible application of ES cells is to generate new cardiomyocytes for the cell replacement therapy of heart failure (e.g., chronic heart failure), by first producing cardiac progenitors, from, e.g., hESCs, and then further differentiating the cardiac progenitors into at least one immature cardiomyocyte or precursor thereof, and then further differentiating the at least one immature cardiomyocyte or precursor thereof into a cardiomyocyte (e.g., mature cardiomyocyte).

hESC cells, are described, for example, by Cowan et al. (N Engl. J. Med. 350:1353, 2004) and Thomson et al. (Science 282:1145, 1998); embryonic stem cells from other primates, Rhesus stem cells (Thomson et al., Proc. Natl. Acad. Sci. USA 92:7844, 1995), marmoset stem cells (Thomson et al., Biol. Reprod. 55:254, 1996) and human embryonic germ (hEG) cells (Shamblott et al., Proc. Natl. Acad. Sci. USA 95:13726, 1998) may also be used in the methods disclosed herein. mESCs, are described, for example, by Tremml et al. (Curr Protoc Stem Cell Biol. Chapter 1: Unit 1C.4, 2008). The stem cells may be, for example, unipotent, totipotent, multipotent, or pluripotent. In some examples, any cells of primate origin that are capable of producing progeny that are derivatives of at least one germinal layer, or all three germinal layers, may be used in the methods disclosed herein.

In certain examples, ES cells may be isolated, for example, as described in Cowan et al. (N Engl. J. Med. 350:1353, 2004) and U.S. Pat. No. 5,843,780 and Thomson et al., Proc. Natl. Acad. Sci. USA 92:7844, 1995. For example, hESCs cells can be prepared from human blastocyst cells using the techniques described by Thomson et al. (U.S. Pat. No. 6,200,806; Science 282:1145, 1998; Curr. Top. Dev. Biol. 38:133 ff., 1998) and Reubinoff et al, Nature Biotech. 18:399, 2000. Equivalent cell types to hESCs include their pluripotent derivatives, such as primitive ectoderm-like (EPL) cells, as outlined, for example, in WO 01/51610 (Bresagen). hESCs can also be obtained from human pre-implantation embryos.

Alternatively, in vitro fertilized (IVF) embryos can be used, or one-cell human embryos can be expanded to the blastocyst stage (Bongso et al., Hum Reprod 4:706, 1989). Embryos are cultured to the blastocyst stage in G1.2 and G2.2 medium (Gardner et al., Fertil. Steril. 69:84, 1998). The zona pellucida is removed from developed blastocysts by brief exposure to pronase (Sigma). The inner cell masses can be isolated by immunosurgery, in which blastocysts are exposed to a 1:50 dilution of rabbit anti-human spleen cell antiserum for 30 min, then washed for 5 min three times in DMEM, and exposed to a 1:5 dilution of Guinea pig complement (Gibco) for 3 min (Solter et al., Proc. Natl. Acad. Sci. USA 72:5099, 1975). After two further washes in DMEM, lysed trophectoderm cells are removed from the intact inner cell mass (ICM) by gentle pipetting, and the ICM plated on mEF feeder layers. After 9 to 15 days, inner cell mass-derived outgrowths can be dissociated into clumps, either by exposure to calcium and magnesium-free phosphate-buffered saline (PBS) with 1 mM EDTA, by exposure to dispase or trypsin, or by mechanical dissociation with a micropipette; and then replated on mEF in fresh medium. Growing colonies having undifferentiated morphology can be individually selected by micropipette, mechanically dissociated into clumps, and replated. ES-like morphology is characterized as compact colonies with apparently high nucleus to cytoplasm ratio and prominent nucleoli. Resulting hESCs can then be routinely split every 1-2 weeks, for example, by brief trypsinization, exposure to Dulbecco's PBS (containing 2 mM EDTA), exposure to type IV collagenase (about 200 U/mL; Gibco) or by selection of individual colonies by micropipette. In some examples, clump sizes of about 50 to 100 cells are optimal. mESCs cells can be prepared from using the techniques described by e.g., Conner et al. (Curr. Prot. in Mol. Biol. Unit 23.4, 2003).

Embryonic stem cells can be isolated from blastocysts of members of the primate species (U.S. Pat. No. 5,843,780; Thomson et al., Proc. Natl. Acad. Sci. USA 92:7844, 1995). Human embryonic stem (hES) cells can be prepared from human blastocyst cells using the techniques described by Thomson et al. (U.S. Pat. No. 6,200,806; Science 282:1145, 1998; Curr. Top. Dev. Biol. 38:133 ff., 1998) and Reubinoff et al, Nature Biotech. 18:399, 2000. Equivalent cell types to hES cells include their pluripotent derivatives, such as primitive ectoderm-like (EPL) cells, as outlined in WO 01/51610 (Bresagen).

Alternatively, in some embodiments, hES cells can be obtained from human preimplantation embryos. Alternatively, in vitro fertilized (IVF) embryos can be used, or one-cell human embryos can be expanded to the blastocyst stage (Bongso et al., Hum Reprod 4:706, 1989). Embryos are cultured to the blastocyst stage in G1.2 and G2.2 medium (Gardner et al., Fertil. Steril. 69:84, 1998). The zona pellucida is removed from developed blastocysts by brief exposure to pronase (Sigma). The inner cell masses are isolated by immunosurgery, in which blastocysts are exposed to a 1:50 dilution of rabbit anti-human spleen cell antiserum for 30 min, then washed for 5 min three times in DMEM, and exposed to a 1:5 dilution of Guinea pig complement (Gibco) for 3 min (Solter et al., Proc. Natl. Acad. Sci. USA 72:5099, 1975). After two further washes in DMEM, lysed trophectoderm cells are removed from the intact inner cell mass (ICM) by gentle pipetting, and the ICM plated on mEF feeder layers.

After 9 to 15 days, inner cell mass-derived outgrowths are dissociated into clumps, either by exposure to calcium and magnesium-free phosphate-buffered saline (PBS) with 1 mM EDTA, by exposure to dispase or trypsin, or by mechanical dissociation with a micropipette; and then replated on mEF in fresh medium. Growing colonies having undifferentiated morphology are individually selected by micropipette, mechanically dissociated into clumps, and replated. ES-like morphology is characterized as compact colonies with apparently high nucleus to cytoplasm ratio and prominent nucleoli. Resulting ES cells are then routinely split every 1-2 weeks by brief trypsinization, exposure to Dulbecco's PBS (containing 2 mM EDTA), exposure to type IV collagenase (200 U/mL; Gibco) or by selection of individual colonies by micropipette. Clump sizes of about 50 to 100 cells are optimal.

In some embodiments, human Embryonic Germ (hEG) cells are pluripotent stem cells which can be used in the methods as disclosed herein to differentiate into primitive endoderm cells. hEG cells can be used be prepared from primordial germ cells present in human fetal material taken about 8-11 weeks after the last menstrual period. Suitable preparation methods are described in Shamblott et al., Proc. Natl. Acad. Sci. USA 95:13726, 1998 and U.S. Pat. No. 6,090,622, which is incorporated herein in its entirety by reference.

Briefly, genital ridges processed to form disaggregated cells. EG growth medium is DMEM, 4500 mg/L D-glucose, 2200 mg/L mM NaHCO₃; 15% ES qualified fetal calf serum (BRL); 2 mM glutamine (BRL); 1 mM sodium pyruvate (BRL); 1000-2000 U/mL human recombinant leukemia inhibitory factor (LIF, Genzyme); 1-2 ng/mL human recombinant bFGF (Genzyme); and 10 μM forskolin (in 10% DMSO). Ninety-six well tissue culture plates are prepared with a sub-confluent layer of feeder cells (e.g., STO cells, ATCC No. CRL 1503) cultured for 3 days in modified EG growth medium free of LIF, bFGF or forskolin, inactivated with 5000 rad γ-irradiation ^{˜}0.2 mL of primary germ cell (PGC) suspension is added to each of the wells. The first passage is done after 7-10 days in EG growth medium, transferring each well to one well of a 24-well culture dish previously prepared with irradiated STO mouse fibroblasts. The cells are cultured with daily replacement of medium until cell morphology consistent with EG cells is observed, typically after 7-30 days or 1-4 passages.

In certain examples, the stem cells can be undifferentiated (e.g., a cell not committed to a specific linage) prior to exposure to at least one cardiomyocyte maturation factor according to the methods as disclosed herein, whereas in other examples it may be desirable to differentiate the stem cells to one or more intermediate cell types prior to exposure of the at least one cardiomyocyte maturation factor(s) described herein. For example, the stem cells may display morphological, biological or physical characteristics of undifferentiated cells that can be used to distinguish them from differentiated cells of embryo or adult origin. In some examples, undifferentiated cells may appear in the two dimensions of a microscopic view in colonies of cells with high nuclear/cytoplasmic ratios and prominent nucleoli. The stem cells may be themselves (for example, without substantially any undifferentiated cells being present) or may be used in the presence of differentiated cells. In certain examples, the stem cells may be cultured in the presence of suitable nutrients and optionally other cells such that the stem cells can grow and optionally differentiate. For example, embryonic fibroblasts or fibroblast-like cells may be present in the culture to assist in the growth of the stem cells. The fibroblast may be present during one stage of stem cell growth but not necessarily at all stages. For example, the fibroblast may be added to stem cell cultures in a first culturing stage and not added to the stem cell cultures in one or more subsequent culturing stages.

Stem cells used in all aspects of the present invention can be any cells derived from any kind of tissue (for example embryonic tissue such as fetal or pre-fetal tissue, or adult tissue), which stem cells have the characteristic of being capable under appropriate conditions of producing progeny of different cell types, e.g., derivatives of all of at least one of the 3 germinal layers (endoderm, mesoderm, and ectoderm). These cell types may be provided in the form of an established cell line, or they may be obtained directly from primary embryonic tissue and used immediately for differentiation. Included are cells listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Scoul National University); HSF-1, HSF-6 (University of California at San Francisco); and H1, H7, H9, H13, H14 (Wisconsin Alumni Research Foundation (WiCell Research Institute)). In some embodiments, the source of human stem cells or pluripotent stem cells used for chemically-induced differentiation into mature cardiomyocytes did not involve destroying a human embryo.

In another embodiment, the stem cells can be isolated from tissue including solid tissue. In some embodiments, the tissue is skin, fat tissue (e.g., adipose tissue), muscle tissue, heart or cardiac tissue. In other embodiments, the tissue is for example but not limited to, umbilical cord blood, placenta, bone marrow, or chondral.

Stem cells of interest also include embryonic cells of various types, exemplified by human embryonic stem (hES) cells, described by Thomson et al. (1998) Science 282:1145; embryonic stem cells from other primates, such as Rhesus stem cells (Thomson et al. (1995) Proc. Natl. Acad. Sci. USA 92:7844); marmoset stem cells (Thomson et al. (1996) Biol. Reprod. 55:254); and human embryonic germ (hEG) cells (Shambloft et al., Proc. Natl. Acad. Sci. USA 95:13726, 1998). Also of interest are lineage committed stem cells, such as mesodermal stem cells and other early cardiogenic cells (see Reyes et al. (2001) Blood 98:2615-2625; Eisenberg & Bader (1996) Circ Res. 78 (2): 205-16; etc.) The stem cells may be obtained from any mammalian species, e.g. human, equine, bovine, porcine, canine, feline, rodent, e.g. mice, rats, hamster, primate, etc. In some embodiments, a human embryo was not destroyed for the source of pluripotent cell used on the methods and compositions as disclosed herein.

ES cells are considered to be undifferentiated when they have not committed to a specific differentiation lineage. Such cells display morphological characteristics that distinguish them from differentiated cells of embryo or adult origin. Undifferentiated ES cells are easily recognized by those skilled in the art, and typically appear in the two dimensions of a microscopic view in colonies of cells with high nuclear/cytoplasmic ratios and prominent nucleoli. Undifferentiated ES cells express genes that may be used as markers to detect the presence of undifferentiated cells, and whose polypeptide products may be used as markers for negative selection. For example, see U.S. application Ser. No. 2003/0224411 A1; Bhattacharya (2004) Blood 103 (8): 2956-64; and Thomson (1998), supra., each herein incorporated by reference. Human ES cell lines express cell surface markers that characterize undifferentiated nonhuman primate ES and human EC cells, including stage-specific embryonic antigen (SSEA)-3, SSEA-4, TRA-1-60, TRA-1-81, and alkaline phosphatase. The globo-series glycolipid GL7, which carries the SSEA-4 epitope, is formed by the addition of sialic acid to the globo-series glycolipid GbS, which carries the SSEA-3 epitope. Thus, GL7 reacts with antibodies to both SSEA-3 and SSEA-4. The undifferentiated human ES cell lines did not stain for SSEA-1, but differentiated cells stained strongly for SSEA-I. Methods for proliferating hES cells in the undifferentiated form are described in WO 99/20741, WO 01/51616, and WO 03/020920.

A mixture of cells from a suitable source of endothelial, muscle, and/or neural stem cells can be harvested from a mammalian donor by methods known in the art. A suitable source is the hematopoietic microenvironment. For example, circulating peripheral blood, preferably mobilized (i.e., recruited), may be removed from a subject. Alternatively, bone marrow may be obtained from a mammal, such as a human patient, undergoing an autologous transplant. In some embodiments, stem cells can be obtained from the subjects adipose tissue, for example using the CELUTION™ SYSTEM from Cytori, as disclosed in U.S. Pat. Nos. 7,390,484 and 7,429,488 which is incorporated herein in its entirety by reference.

In some embodiments, human umbilical cord blood cells (HUCBC) are useful in the methods as disclosed herein. Human UBC cells are recognized as a rich source of hematopoietic and mesenchymal progenitor cells (Broxmeyer et al., 1992 Proc. Natl. Acad. Sci. USA 89:4109-4113). Previously, umbilical cord and placental blood were considered a waste product normally discarded at the birth of an infant. Cord blood cells are used as a source of transplantable stem and progenitor cells and as a source of marrow repopulating cells for the treatment of malignant diseases (i.e., acute lymphoid leukemia, acute myeloid leukemia, chronic myeloid leukemia, myelodysplastic syndrome, and nucroblastoma) and non-malignant diseases such as Fanconi's anemia and aplastic anemia (Kohli-Kumar et al., 1993 Br. J. Haematol. 85:419-422; Wagner et al., 1992 Blood 79; 1874-1881; Lu et al., 1996 Crit. Rev. Oncol. Hematol 22:61-78; Lu et al., 1995 Cell Transplantation 4:493-503). A distinct advantage of HUCBC is the immature immunity of these cells that is very similar to fetal cells, which significantly reduces the risk for rejection by the host (Taylor & Bryson, 1985 J. Immunol. 134:1493-1497). Human umbilical cord blood contains mesenchymal and hematopoietic progenitor cells, and endothelial cell precursors that can be expanded in tissue culture (Broxmeyer et al., 1992 Proc. Natl. Acad. Sci. USA 89:4109-4113; Kohli-Kumar et al., 1993 Br. J. Haematol. 85:419-422; Wagner et al., 1992 Blood 79; 1874-1881; Lu et al., 1996 Crit. Rev. Oncol. Hematol 22:61-78; Lu et al., 1995 Cell Transplantation 4:493-503; Taylor & Bryson, 1985 J. Immunol. 134:1493-1497; Broxmeyer, 1995 Transfusion 35:694-702; Chen et al., 2001 Stroke 32:2682-2688; Nieda et al., 1997 Br. J. Haematology 98:775-777; Erices et al., 2000 Br. J. Haematology 109:235-242). The total content of hematopoietic progenitor cells in umbilical cord blood equals or exceeds bone marrow, and in addition, the highly proliferative hematopoietic cells are eightfold higher in HUCBC than in bone marrow and express hematopoietic markers such as CD14, CD34, and CD45 (Sanchez-Ramos et al., 2001 Exp. Neur. 171:109-115; Bicknese et al., 2002 Cell Transplantation 11:261-264; Lu et al., 1993 J. Exp Med. 178:2089-2096).

In another embodiment, pluripotent cells are cells in the hematopoietic microenvironment, such as the circulating peripheral blood, preferably from the mononuclear fraction of peripheral blood, umbilical cord blood, bone marrow, fetal liver, or yolk sac of a mammal. The stem cells, especially neural stem cells, may also be derived from the central nervous system, including the meninges.

In another embodiment, pluripotent cells are present in embryoid bodies are formed by harvesting ES cells with brief protease digestion, and allowing small clumps of undifferentiated human ESCs to grow in suspension culture. Differentiation is induced by withdrawal of conditioned medium. The resulting embryoid bodies are plated onto semi-solid substrates. Formation of differentiated cells may be observed after around about 7 days to around about 4 weeks. Viable differentiating cells from in vitro cultures of stem cells are selected for by partially dissociating embryoid bodies or similar structures to provide cell aggregates. Aggregates comprising cells of interest are selected for phenotypic features using methods that substantially maintain the cell to cell contacts in the aggregate.

In an alternative embodiment, the stem cells can be reprogrammed stem cells, such as stem cells derived from somatic or differentiated cells. In such an embodiment, the de-differentiated stem cells can be for example, but not limited to, neoplastic cells, tumor cells and cancer cells or alternatively induced reprogrammed cells such as induced pluripotent stem cells or iPS cells. Reprogrammed iPS cells may be obtained using any method known to those of skill in the art. For example, reprogrammed iPS cells may be obtained using one or more transcription factors. In one embodiment, iPSC cells are obtained via reprogramming, e.g., reprogramming somatic cells, using one or more transcription factors including, but not limited to, Oct4, Sox2, Klf4, and c-Myc. Additional methods for making reprogrammed iPS cells are described in WO 2013/177133 and WO 2022/204567, both of which are incorporated herein by reference.

### Cloning and Cell Culture

Illustrative methods for molecular genetics and genetic engineering that may be used in the technology described herein may be found, for example, in current editions of Molecular Cloning: A Laboratory Manual, (Sambrook et al., Cold Spring Harbor); Gene Transfer Vectors for Mammalian Cells (Miller & Calos eds.); and Current Protocols in Molecular Biology (F. M. Ausubel et al. eds., Wiley & Sons). Cell biology, protein chemistry, and antibody techniques can be found, for example, in Current Protocols in Protein Science (J. E. Colligan et al. eds., Wiley & Sons); Current Protocols in Cell Biology (J. S. Bonifacino et al., Wiley & Sons) and Current protocols in Immunology (J. E. Colligan et al. eds., Wiley & Sons). Illustrative reagents, cloning vectors, and kits for genetic manipulation may be commercially obtained, for example, from BioRad, Stratagene, Invitrogen, ClonTech, and Sigma-Aldrich Co.

Suitable cell culture methods may be found, for example, in the current edition of Culture of Animal Cells: A Manual of Basic Technique (R. I. Freshney ed., Wiley & Sons); General Techniques of Cell Culture (M. A. Harrison & I. F. Rac, Cambridge Univ. Press), and Embryonic Stem Cells: Methods and Protocols (K. Turksen ed., Humana Press). Suitable tissue culture supplies and reagents are commercially available, for example, from Gibco/BRL, Nalgene-Nunc International, Sigma Chemical Co., and ICN Biomedicals.

Pluripotent stem cells can be propagated by one of ordinary skill in the art and continuously in culture, using culture conditions that promote proliferation without promoting differentiation. Exemplary serum-containing ES medium is made with 80% DMEM (such as Knock-Out DMEM, Gibco), 20% of either defined fetal bovine serum (FBS, Hyclone) or serum replacement (WO 98/30679), 1% non-essential amino acids, 1 mM L-glutamine, and 0.1 mM β-mercaptoethanol. Just before use, human bFGF is added to 4 ng/ml (WO 99/20741, Geron Corp.). Traditionally, ES cells are cultured on a layer of feeder cells, typically fibroblasts derived from embryonic or fetal tissue.

Alternatively, pluripotent SCs can be maintained in an undifferentiated state even without feeder cells. The environment for feeder-free cultures includes a suitable culture substrate, particularly an extracellular matrix such as MATRIGEL® (gelatinous protein mixture) or laminin. Typically, enzymatic digestion is halted before cells become completely dispersed (˜5 min with collagenase IV). Clumps of ˜10 to 2,000 cells are then plated directly onto the substrate without further dispersal.

### Pharmaceutical Compositions

Some embodiments of the present invention relate to compositions, such as cell cultures or cell populations comprising stem cell-derived cells or precursors thereof. For example, compositions of the present invention may include cardiomyocytes, beta cells, satellite cells, alpha cells, delta cells, hematopoietic cells, dermal cells, enterochromaffin cells, endothelial cells, etc. or precursors or progenitors thereof. In some embodiments, a composition comprises any cell that has been derived in vitro from a stem cell, such as a pluripotent stem cell. In certain aspects, the stem cell-derived cell has reached a stage of partial maturity. The stem cell-derived cell may be administered to a subject in vivo to complete the maturation, differentiation, and/or engraftment process of the cell, e.g., to gain full functionality similar to an endogenous cell. In some aspects, the in vivo maturation of the stem cell-derived cells requires one or more weeks or months upon administration of the cell. Maturation, differentiation, and/or engraftment of the stem cell-derived cells or precursors thereof may be measured by the appropriate guideposts dependent on the cell type, as known to those of skill in the art.

In some aspects, the compositions further comprise one or more self-assembling peptides (SAP). Self-assembling peptides are biomedical materials that exhibit unique structures that may be formed in response to various physiochemical characteristics. In some aspects, the self-assembling peptides form ordered structures, such as scaffolds to support cell and/or tissue generation and growth. In some embodiments, self-assembling peptides recruit or encourage blood cell and/or blood vessel formation, e.g., by mimicking the extracellular matrix and facilitating cell adhesion, migration and proliferation. In one aspect, the contacting of a stem cell-derived cell with a self-assembling peptide results in the maturation of the stem cell-derived cell, e.g., as a result of the increased blood cells that occur from administration of the SAP. Non-limiting examples of self-assembly peptides that may assist in the maturation of a stem cell-derived cell include RADA16, IEIK13, KL.D12, and QLEL12

In certain aspects a composition comprises a population of stem cell-derived cardiomyocytes (e.g., pluripotent stem cell-derived cardiomyocytes, such as human induced pluripotent stem cell-derived cardiomyocytes (hiPSC-CMs)). In some aspects, a composition comprises a population of matured hiPSC-CMs. In some embodiments, the cardiomyocytes have been derived from at least one immature cardiomyocyte or a precursor thereof. In some aspects, the hiPSC-CMs are not fully mature cardiomyocytes, although they may exhibit one or more features of a mature cardiomyocyte. In some embodiments, the stem cell-derived cardiomyocytes are derived from pluripotent stem cells, e.g., human-induced pluripotent stem cells. In some embodiments, the cardiomyocytes are mammalian cells, and in a preferred embodiment, such cardiomyocytes are human cardiomyocytes.

In some embodiments, stem cell-derived cells (e.g., hiPSC-CMs) are obtained by any methods known to those of skill in the art. The stem cell-derived cells can be produced according to any suitable culturing protocol or series of culturing protocols to differentiate a stem cell or pluripotent cell to a desired stage of differentiation. In some embodiments, the stem cell-derived cells (hiPSC-CMs) or the precursors thereof are produced by culturing at least one pluripotent cell for a period of time and under conditions suitable for the at least one pluripotent cell to differentiate into the differentiated cell type (e.g., cardiomyocytes or the precursors thereof). Culturing of the cells may occur under standard conditions of temperature, pH, and other environmental conditions, e.g., as adherent cells in tissue culture plates or in 3D culture in Erlenmeyer flasks at 37° C. in an atmosphere containing 5-10% CO₂. Isolation and culture methods for various cells are well within the abilities of one skilled in the art.

The stem cell-derived cells are differentiated in vitro from any starting cell as the invention is not intended to be limited by the starting cell from which the stem cell-derived cells are derived. For example, starting cells for cardiomyocytes include, without limitation, immature cardiomyocytes or any precursor thereof such as a cardiac progenitor cell, a pluripotent stem cell, an embryonic stem cell, and induced pluripotent stem cell. In some embodiments, the stem cell-derived cells are differentiated in vitro from a reprogrammed cell, a partially reprogrammed cell (i.e., a somatic cell, e.g., a fibroblast which has been partially reprogrammed such that it exists in an intermediate state between an induced pluripotency cell and the somatic cell from which it has been derived), or a transdifferentiated cell. In some embodiments, a cardiomyocyte is differentiated in vitro from a precursor selected from the group consisting of a cardiac progenitor cell and a pluripotent stem cell. In some embodiments, the pluripotent stem cell is selected from the group consisting of an embryonic stem cell and induced pluripotent stem cell. In some embodiments, the cardiomyocyte or the pluripotent stem cell from which the cardiomyocyte is derived is human. In some embodiments, the cardiomyocyte is human.

The disclosure contemplates methods in which cardiomyocytes are generated according to the methods described herein from iPS cells derived from cells extracted or isolated from individuals suffering from a disease (e.g., heart failure, or a cardiac-related disorder), and those cardiomyocytes are compared to normal cardiomyocytes from healthy individuals not having the disease to identify differences between the cardiomyocytes and normal cardiomyocytes which could be useful as markers for disease (e.g., epigenetic and/or genetic). In some embodiments, cardiomyocytes are obtained from an individual suffering from heart failure and compared to normal cardiomyocytes, and then the cardiomyocytes are reprogrammed to iPS cells and the iPS cells are analyzed for genetic and/or epigenetic markers which are present in the cardiomyocytes obtained from the individual suffering from heart failure but not present in the normal cardiomyocytes, to identify markers (e.g., pre-heart failure). In some embodiments, the iPS cells and/or cardiomyocytes derived from patients are used to screen for agents (e.g., agents which are able to modulate genes contributing to a heart failure phenotype).

hiPSC-CMs of use herein can be derived from any source or generated in accordance with any suitable protocol. In some aspects, pluripotent stem cells, e.g., iPSCs or hESCs, are differentiated to hiPSC-CMs. In some embodiments, pluripotent stem cells are differentiated to hiPSC-CMs using a differentiation protocol described by Lian et al. (Nat Protoc. 2012; 8 (1): 162-175), which is incorporated herein by reference. In some embodiments, the differentiation protocol described by Lian was modified as described herein. In some embodiments, pluripotent stem cells are contacted with one or more small molecules to manipulate the Wnt pathway, and thereby differentiate the pluripotent stem cells into hiPSC-CMs. In some aspects, the one or more small molecules are selected from the group consisting of CHIR 99021 and IWP4. In some embodiments, a population of pluripotent stem cells is contacted with a first Wnt pathway modulator (e.g., CHIR 99021), and is then contacted with a second Wnt pathway modulator (e.g., IWP4).

In some embodiments, the hiPSC-CMs or precursors thereof are maintained in culture by methods known by one of ordinary skill in the art, and in some embodiments, propagated prior to being matured into cardiomyocytes by the methods as disclosed herein. In some embodiments, the hiPSC-CMs are cultured in 2D or 3D culture. In some embodiments, the hiPSC-CMs are immature cardiomyocytes. Additional methods for producing cardiomyocytes are described by U.S. Pat. No. 9,452,201; WO 2014/200339; WO 2017/039445, WO 2020/247957, WO 2022/221051, and WO 2023/086599, which are incorporated herein by reference.

Stem cell-derived cells or precursors thereof can be from any mammalian species, with non-limiting examples including a murine, bovine, simian, porcine, equine, ovine, or human cell. For clarity and simplicity, the description of the methods herein refers to mammalian stem cell-derived cells or precursor thereof, but it should be understood that all of the methods described herein can be readily applied to other cell types of stem cell-derived cells or precursors thereof. In some embodiments, the stem cell-derived cells or precursors thereof are derived from a human individual. In some embodiments, the compositions comprise human stem cell-derived cells that are non-recombinant cells. In such embodiments, the compositions are devoid of or substantially free of recombinant human stem cell-derived cells.

In some embodiments, a composition, such as a pharmaceutical composition, comprises cardiomyocytes (e.g., hiPSC-CMs) and one or more self-assembling peptides. Non-limiting examples of self-assembling peptides include RADA16, IEIK13, KLD12, and QLEL12. In an exemplary embodiment, a self-assembling peptide (SAP) comprises RADA16. In some embodiments, the pharmaceutical composition is a suspension of a SAP and hiPSC-CMs. The pharmaceutical composition may further comprise a pharmaceutically acceptable carrier or excipient. For example, the pharmaceutical composition may comprise cell medium (e.g., RPMI cell medium), B27, and/or insulin (RBI). In some embodiments, RBI is further supplemented with one or more pro-survival cocktails and/or endothelial growth factors (PVbBB). For example, RBI may be supplemented with one or more of ZVAD, Bcl-XL BH4, Cyclosporine A, IGF-1, Pnacidil, VEGF, bFGF, and PDGF-BB.

In some embodiments, the contact or co-culture of hiPSC-CMs with a SAP results in maturation of the hiPSC-CMs, e.g., to form mature cardiomyocytes. In some embodiments, the contact of hiPSC-CMs with a SAP occurs upon administration to a subject. For example, the SAP may recruit blood vessels and/or blood cells to the site of administration, which thereby facilitate the maturation of the hiPSC-CMs to mature cardiomyocytes. The mature cardiomyocytes may resemble endogenous mature cardiomyocytes in form and function, but nevertheless are distinct from native cardiomyocytes. In some embodiments, the morphology of the cardiomyocytes resembles the morphology of endogenous cardiomyocytes. In some embodiments, the cardiomyocytes are electrically mature. In some embodiments, the cardiomyocytes are contractility mature. In some embodiments, the cardiomyocytes are metabolically mature. In some embodiments, the cardiomyocytes exhibit enhanced sarcomere organization. In some embodiments, the cardiomyocytes exhibit adult-like gene expression profiles. In some embodiments, the cardiomyocytes exhibit improved vascularization in the transplanted or administration site.

In some embodiments, the cardiomyocytes are mature. In some embodiments, the cardiomyocytes exhibit increased expression of sarcomeric proteins (e.g., TNNT2, TNNI1, and/or TNNI3). In some embodiments, the cardiomyocytes exhibit decreased beating rate as compared to fetal or immature cardiomyocytes. In some embodiments, the cardiomyocytes exhibit decreased automaticity. In some embodiments, the cardiomyocytes exhibit increased mean beat amplitude, mean spike amplitude, and/or upstroke velocity as compared to immature cardiomyocytes. In some embodiments, the cardiomyocytes exhibit increased oxygen consumption and/or respiratory reserve as compared to immature cardiomyocytes. In some embodiments, the cardiomyocytes exhibit increased expression of one or more markers selected from the group consisting of TNNT2, TNNI1, TNNI3, and Cx43/GJA1 and KCNJ2 as compared to immature cardiomyocytes.

In some embodiments, hiPSC-CMs contacted with a SAP express one or more of MYH7, MYH6, TNNI3, TNNI1, GJA1, MYL2 and KCNJ2. In some aspects, the hiPSC-CMs contacted with a SAP exhibit increased expression of MYH7/6 as compared to control hiPSC-CMs. In some aspects, the hiPSC-CMs contacted with a SAP exhibit increased expression of GJA1 as compared to control hiPSC-CMs. In some aspects, the hiPSC-CMs contacted with a SAP exhibit increased expression of MYL2 as compared to control hiPSC-CMs. In some aspects, the hiPSC-CMs contacted with a SAP exhibit increased expression of KCNJ2 as compared to control hiPSC-CMs. In some embodiments, the hiPSC-CMs contacted with a SAP exhibit 0.1 to 100 fold, 1 to 100 fold, 5 to 100 fold, 10 to 100 fold, 25 to 100 fold, 0.1 to 75 fold, 1 to 75 fold, 5 to 75 fold, 10 to 75 fold, 25 to 75 fold, 0.1 to 50 fold, 1 to 50 fold, 5 to 50 fold, 10 to 50 fold, 25 to 50 fold, 0.1 to 25 fold, 1 to 25 fold, 5 to 25 fold, 10 to 25 fold, 0.1 to 10 fold, 1 to 10 fold, 5 to 10 fold, 0.1 to 5 fold, 1 to 5 fold, or 0.1 to 1 fold increased expression of at least one marker comprising MYH7/6, TNNI3/1, GJA1, MYL2 and KCNJ2. In some embodiments, the hiPSC-CMs contacted with a SAP exhibit at least a 0.1-fold, 0.2-fold, 0.3-fold, 0.4-fold, 0.5-fold, 0.6-fold, 0.7-fold, 0.8-fold, 0.9-fold, 1-fold, 1.5-fold, 2-fold, 2.5-fold, 3-fold, 3.5-fold, 4-fold, 4.5-fold, 5-fold, 10-fold, 25-fold, 50-fold, or in some aspects greater than a 50-fold increased expression of at least one marker comprising MYH7/6, TNNI3/1, GJA1, MYL2 and KCNJ2.

In some aspects, the hiPSC-CMs contacted with a SAP exhibit decreased expression of HCN4 as compared to control hiPSC-CMs. In some embodiments, the hiPSC-CMs contacted with a SAP exhibit 1 to 99%, 5 to 99%, 10 to 99%, 25 to 99%, 50 to 99%, 60 to 99%, 75 to 99%, 1 to 75%, 5 to 75%, 10 to 75%, 15 to 75%, 20 to 75%, 25 to 75%, 30 to 75%, 35 to 75%, 40 to 75%, 45 to 75%, 50 to 75%, 55 to 75%, 60 to 75%, 1 to 50%, 5 to 50%, 10 to 50%, 15 to 50%, 20 to 50%, 25 to 50%, 30 to 50%, 35 to 50%, 40 to 50%, 1 to 25%, 5 to 25%, 10 to 25%, 15 to 25%, 20 to 25%, 1 to 15%, 5 to 15%, 10 to 15%, 5 to 10%, or 1 to 5% decreased expression of HCN4.

In some embodiments, hiPSC-CMs contacted with a SAP are engrafted and/or vascularized with a subject's heart tissue upon administration or implantation. In some aspects, the ratio of vascularization area (e.g., CD31 positive area) upon administration of hiPSC-CMs with a SAP is increased as compared to administration of hiPSC-CMs alone. In some aspects, the proportion of GSL I-positive vessel area was increased upon administration of hiPSC-CMs with a SAP as compared to administration of hiPSC-CMs alone. In some embodiments, vascularization is maintained is the subject for at least 7 days, 14 days, 21 days, 30 days, 1 month, 2 months, 3 months, 4 months, 5 months, or 6 months. In some embodiments, the administration of hiPSC-CMs and a SAP to a subject results in improved sarcomere organization as compared to administration of hiPSC-CMs alone.

In some embodiments, the administration of hiPSC-CMs and a SAP to a subject results in improved and/or increased sarcomere organization as compared to administration of hiPSC-CMs alone. In some aspects, sarcomere organization is improved in the subject for a period of at least 7 days, 14 days, 21 days, 30 days, 1 month, 2 months, 3 months, 4 months, 5 months, or 6 months. In some embodiments, the administration of hiPSC-CMs and a SAP to a subject results in increased sarcomere length as compared to administration of hiPSC-CMs alone. In some aspects, the sarcomere length is increased in the subject at least 7 days, 14 days, 21 days, 30 days, 1 month, 2 months, 3 months, 4 months, 5 months, or 6 months after administration of hiPSC-CMs and a SAP.

In some embodiments, the hiPSC-CMs contacted with a SAP exhibit decreased automaticity, as compared to control hiPSC-CMs. In some aspects, upon administration to a subject, hiPSC-CMs administered with a SAP exhibited increased reduction in automaticity, as compared to the administration of hiPSC-CMs alone. In some aspects, upon administration to a subject, hiPSC-CMs administered with a SAP exhibited automaticity spikes at a slower rate comparable to sinus spikes, as compared to the administration of hiPSC-CMs alone. In some aspects, the electrical functions of hiPSC-CMs administered to a subject with or without SAP, e.g., administered or implanted to the heart tissue of a subject, are monitored and measured using flexible electronics to enable high-resolution electrophysiological recordings. For example, mesh nanoelectronics may be used to monitor electrophysiological integration of administered hiPSC-CMs, e.g., by monitoring cellular resolution voltage signals at injection and reference sites. Exemplary flexible mesh nanoelectronics as used herein are described in WO 2020/263772, which is incorporated herein by reference.

### Confirmation of the Presence and the Identification of Cardiomyocytes

One can use any means common to one of ordinary skill in the art to confirm the presence of a mature stem cell-derived cell (e.g., mature cardiomyocyte) as compared to the presence of an immature stem cell-derived cell (e.g., immature cardiomyocyte). One can use any means common to one of ordinary skill in the art to confirm the presence of a cardiomyocyte, e.g. a mature cardiomyocyte produced by the contacting of a hiPSC-CM or precursor thereof with a SAP as described herein.

In some embodiments, the presence of cardiomyocyte markers can be assessed by detecting the presence or absence of one or more markers indicative of an endogenous cardiomyocyte. In some embodiments, the method can include detecting the positive expression (e.g., the presence) of a marker for cardiomyocytes. In some embodiments the method can include detecting the positive expression of one or more sarcomeric proteins (e.g., cardiac troponin T (TNNT2), cardiac troponin 1 (TNNI1), or cardiac troponin I (TNNI3)). In some embodiments the method can include detecting the positive expression of Cx43/GJA1. In some embodiments, the marker can be detected using a reagent, e.g., a reagent for the detection of TNNT2, TNNI1, TNNI3, GJA1/Cx43, or MYL2. Cardiomyocytes can also be characterized by the down-regulation of specific markers.

A reagent for a marker can be, for example, an antibody against the marker or primers for a RT-PCR or PCR reaction, e.g., a semi-quantitative or quantitative RT-PCR or PCR reaction. Such markers can be used to evaluate whether a cardiomyocyte has been produced. The antibody or other detection reagent can be linked to a label, e.g., a radiological, fluorescent (e.g., GFP) or colorimetric label for use in detection. If the detection reagent is a primer, it can be supplied in dry preparation, e.g., lyophilized, or in a solution.

The progression of at least one immature cardiomyocyte to a mature state can be monitored by determining the expression of markers characteristic of mature cardiomyocytes. In some processes, the expression of certain markers is determined by detecting the presence or absence of the marker. Alternatively, the expression of certain markers can be determined by measuring the level at which the marker is present in the cells of a cell population or composition.

As described in connection with monitoring the maturation of a cardiomyocyte from a hiPSC-CM upon contact with a SAP, qualitative or semi-quantitative techniques, such as blot transfer methods and immunocytochemistry, can be used to measure marker expression, using methods commonly known to persons of ordinary skill in the art. Alternatively, marker expression can be accurately quantitated through the use of techniques such as quantitative-PCR by methods ordinarily known in the art. Additionally, techniques for measuring extracellular marker content, such as ELISA, may be utilized.

It is understood that the present invention is not limited to those markers listed as cardiomyocyte markers herein, and the present invention also encompasses markers such as cell surface markers, antigens, and other gene products including ESTs, RNA (including microRNAs and antisense RNA), DNA (including genes and cDNAs), and portions thereof.

In addition, maturation of a cardiomyocyte may be monitored using nanomesh technology, as described herein, to monitor electrophysiological integration of transplanted cardiomyocytes (e.g., hiPSC-CMs). For example, implanted or administered hiPSC-CMs may be monitored using mesh nanoelectronics to capture electrical signal propagation and automaticity over days, weeks, and months after transplantation.

### Compositions and Kits

Described herein are compositions which comprise a stem cell-derived cell described herein (e.g., a hiPSC-CM). In some embodiments, the composition also includes a self-assembling peptide described herein and/or cell culture media.

Also described herein are kits for practicing methods disclosed herein and for maturing cardiomyocytes disclosed herein. Also described herein are kits for treating chronic heart failure and reducing the incidence of ventricular arrhythmias. In one aspect, a kit includes at least one hiPSC-CM or precursor thereof and a self-assembling peptide as described herein.

In some embodiment, the compound in the kit can be provided in a watertight or gas tight container which in some embodiments is substantially free of other components of the kit. The compound can be supplied in more than one container. A self-assembling peptide can be provided in any form, e.g., liquid, dried or lyophilized form. It is preferred that a compound(s) (e.g., SAP) described herein be substantially pure and/or sterile. When a compound(s) described herein is provided in a liquid solution, the liquid solution preferably is an aqueous solution, with a sterile aqueous solution being preferred. When a compound(s) described herein is provided as a dried form, reconstitution generally is by the addition of a suitable solvent. The solvent, e.g., sterile water or buffer, can optionally be provided in the kit.

In some embodiments, the kit further optionally comprises informational material. The informational material can be descriptive, instructional, marketing or other material that relates to the methods described herein and/or the use of a compound(s) described herein for the methods described herein.

The informational material of the kits is not limited in its instruction or informative material. In one embodiment, the informational material can include information about production of the compound, molecular weight of the compound, concentration, date of expiration, batch or production site information, and so forth. In one embodiment, the informational material relates to methods for administering the compound. Additionally, the informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a compound described herein and/or its use in the methods described herein. Of course, the informational material can also be provided in any combination of formats.

In one embodiment, the informational material can include instructions to administer a compound(s) (e.g., a SAP) and/or a population of hiPSC-CMs as described herein in a suitable manner to perform the methods described herein, e.g., in a suitable dose, dosage form, or mode of administration (e.g., a dose, dosage form, or mode of administration described herein) (e.g., to a cell in vitro or a cell in vivo). In another embodiment, the informational material can include instructions to administer a compound(s) described herein to a suitable subject, e.g., a human, e.g., a human having or at risk for a disorder described herein or to a cell in vitro.

In addition to a compound(s) described herein, the composition of the kit can include other ingredients, such as a solvent or buffer, a stabilizer, a preservative, a flavoring agent (e.g., a bitter antagonist or a sweetener), a fragrance or other cosmetic ingredient, and/or an additional agent for treating a condition or disorder described herein. Alternatively, the other ingredients can be included in the kit, but in different compositions or containers than a compound described herein. In such embodiments, the kit can include instructions for admixing a compound(s) described herein and the other ingredients, or for using a compound(s) described herein together with the other ingredients, e.g., instructions on combining the two agents prior to administration.

The kit can include one or more containers for the composition containing a SAP as described herein. In some embodiments, the kit contains separate containers (e.g., two separate containers for the two agents), dividers or compartments for the composition(s) and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a compound described herein. For example, the kit includes a plurality of syringes, ampules, foil packets, or blister packs, each containing a single unit dose of a compound described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is a medical implant device, e.g., packaged for surgical insertion.

The kit can also include a component for the detection of a marker for cardiomyocytes, e.g., for a marker described herein, e.g., a reagent for the detection of mature cardiomyocytes. Or in some embodiments, the kit can also comprise reagents for the detection of negative markers of cardiomyocytes for the purposes of negative selection of mature cardiomyocytes or for identification of cells which do not express these negative markers. The reagents can be, for example, an antibody against the marker or primers for a RT-PCR or PCR reaction, e.g., a semi-quantitative or quantitative RT-PCR or PCR reaction. Such markers can be used to evaluate whether an iPS cell has been produced. If the detection reagent is an antibody, it can be supplied in dry preparation, e.g., lyophilized, or in a solution. The antibody or other detection reagent can be linked to a label, e.g., a radiological, fluorescent (e.g., GFP) or colorimetric label for use in detection. If the detection reagent is a primer, it can be supplied in dry preparation, e.g., lyophilized, or in a solution.

The kit can include hiPSC-CMs for the use as a positive cell type control.

### Methods of Administration

In some embodiments, the cells described herein, e.g., a population of hiPSC-CMs are transplantable, e.g., a population of cardiomyocytes can be administered to a subject, in combination with a self-assembling peptide (SAP). In some embodiments, the subject who is administered a population of cardiomyocytes is the same subject from whom a pluripotent stem cell used to differentiate into a cardiomyocyte was obtained (e.g., for autologous cell therapy). In some embodiments, the subject is a different subject. In some embodiments, a subject is suffering from chronic heart failure, or is a normal subject. For example, the cells for transplantation (e.g., a composition comprising a population of cardiomyocytes) can be a form suitable for transplantation.

The method can further include administering the composition to a subject in need thereof, e.g., a mammalian subject, e.g., a human subject. The source of the cells can be a mammal, preferably a human. The source or recipient of the cells can also be a non-human subject, e.g., an animal model. The term “mammal” includes organisms, which include mice, rats, cows, sheep, pigs, rabbits, goats, horses, monkeys, dogs, cats, and preferably humans. Likewise, transplantable cells can be obtained from any of these organisms, including a non-human transgenic organism. In one embodiment, the transplantable cells are genetically engineered, e.g., the cells include an exogenous gene or have been genetically engineered to inactivate or alter an endogenous gene.

A composition comprising a population of cardiomyocytes can be administered to a subject using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). Negrin et al., Biomaterials, 22 (6): 563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the compounds and compositions delineated herein. In some embodiments, a composition comprising a population of cardiomyocytes and a SAP are administered via injection or using a catheter.

For administration to a subject, a composition produced by the methods as disclosed herein, e.g. a population of cardiomyocytes (e.g., hiPSC-CMs) and a SAP, can be administered to a subject, for example in pharmaceutically acceptable compositions. These pharmaceutically acceptable compositions comprise a therapeutically-effective amount of a population of hiPSC-CMs and a SAP as described above, formulated together with one or more pharmaceutically acceptable carriers (additives) and/or diluents.

As described in detail below, the pharmaceutical compositions of the present invention can be specially formulated for administration in solid or liquid form, including those adapted for the following: (1) parenteral administration, for example, by subcutaneous, intramuscular, intravenous or epidural injection as, for example, a sterile solution or suspension, or sustained-release formulation; (2) topical application, for example, as a cream, ointment, or a controlled-release patch or spray applied to the skin or tissue; or (3) transdermally. Additionally, compounds can be implanted into a patient or injected using a drug delivery system. See, for example, Urquhart, et al., Ann. Rev. Pharmacol. Toxicol. 24:199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids; (23) serum component, such as serum albumin, HDL and LDL; (24) C₂-C₁₂ alcohols, such as ethanol; and (25) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

The phrase “therapeutically-effective amount” as used herein in respect to a population of cells means that amount of relevant cells in a population of cells which is effective for producing some desired therapeutic effect in at least a sub-population of cells in an animal at a reasonable benefit/risk ratio applicable to any medical treatment. For example, an amount of a population of cardiomyocytes administered to a subject that is sufficient to produce a statistically significant, measurable change in at least one symptom of chronic heart failure, such as systolic heart function or incidence of ventricular arrhythmias, etc. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

By “treatment”, “prevention” or “amelioration” of a disease or disorder is meant delaying or preventing the onset of such a disease or disorder, reversing, alleviating, ameliorating, inhibiting, slowing down or stopping the progression or severity of a condition associated with such a disease or disorder. In one embodiment, the symptoms of a disease or disorder are alleviated by at least 5%, at least 10%, at least 20%, at least 30%, at least 40%, or at least 50%.

As used herein, the term “administer” refers to the placement of a composition into a subject by a method or route which results in at least partial localization of the composition at a desired site such that the desired effect is produced. Routes of administration suitable for the methods of the invention include both local and systemic administration. Generally, local administration results in more of the administered cardiomyocytes being delivered to a specific location as compared to the entire body of the subject, whereas systemic administration results in delivery of the cardiomyocytes to essentially the entire body of the subject.

In the context of administering a compound treated cell, the term “administering” also includes transplantation of such a cell in a subject. As used herein, the term “transplantation” refers to the process of implanting or transferring at least one cell to a subject. The term “transplantation” includes, e.g., autotransplantation (removal and transfer of cell(s) from one location on a patient to the same or another location on the same patient), allotransplantation (transplantation between members of the same species), and xenotransplantation (transplantation between members of different species).

Cardiomyocytes or compositions comprising the same can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, ingestion, or topical application. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the compositions are administered by intravenous infusion or injection. In other preferred embodiments, the compositions are administered via a cell patch. In some embodiments, the compositions are administered via a three-dimensional structure (e.g., a matrix or scaffold). In some embodiments, the compositions are administered via a micro-tissue. In some embodiments, the compositions are administered via a syringe. In some embodiments, the compositions are administered via a catheter.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein. The terms, “patient” and “subject” are used interchangeably herein. A subject can be male or female.

Preferably, the subject is a mammal. The mammal can be a human, non-human primate, mouse, rat, dog, cat, horse, or cow, but are not limited to these examples. Mammals other than humans can be advantageously used as subjects that represent animal models of disorders associated with decreased systolic heart function or ventricular arrhythmias. In addition, the methods and compositions described herein can be used to treat domesticated animals and/or pets.

A subject can be one who has been previously diagnosed with or identified as suffering from or having a disorder characterized with decreased systolic heart function or ventricular arrhythmias. A subject may be someone who has been previously diagnosed with or identified as having heart failure (e.g., chronic heart failure). In some aspects, a subject may be someone who has been previously diagnosed with or identified as having a cardiac-related disease or disorder. In some aspects, a subject may be someone who has been previously diagnosed with congenital heart disease (e.g., systolic heart disease or heart disease as a result of tissue engineering).

In some embodiments of the aspects described herein, the method further comprises diagnosing and/or selecting a subject for decreased systolic heart function or ventricular arrhythmias before treating the subject. In some aspects, the method further comprises diagnosing and/or selecting a subject for a cardiac-related disease or disorder before treating the subject. In some aspects, the method further comprises diagnosing and/or selecting a subject for congenital heart disease before treating the subject.

A cardiomyocyte composition described herein can be administered in combination with a mechanical support device (e.g., ventricular assist devices (VADs) or extracorporeal membrane oxygenation (ECMO) systems used to support ventricular recovery), or in combination with cardiac catheterization procedures to revascularize the heart (e.g., stent placement or balloon angioplasty of coronary arteries, or surgical bypass grafting). A cardiomyocyte composition described herein can be co-administrated to a subject in combination with a pharmaceutically active agent. Exemplary pharmaceutically active compound include, but are not limited to, those found in Harrison's Principles of Internal Medicine, 13^{th} Edition, Eds. T. R. Harrison et al. McGraw-Hill N.Y., NY; Physicians' Desk Reference, 50^{th} Edition, 1997, Oradell New Jersey, Medical Economics Co.; Pharmacological Basis of Therapeutics, 8^{th} Edition, Goodman and Gilman, 1990; United States Pharmacopeia, The National Formulary, USP XII NF XVII, 1990; current edition of Goodman and Oilman's The Pharmacological Basis of Therapeutics; and current edition of The Merck Index, the complete content of all of which are herein incorporated in its entirety.

The composition comprising cardiomyocytes and/or a SAP and/or a pharmaceutically active agent can be administrated to the subject in the same pharmaceutical composition or in different pharmaceutical compositions (at the same time or at different times). When administrated at different times, the composition comprising cardiomyocytes and/or the SAP and/or the pharmaceutically active agent can be administered within 5 minutes, 10 minutes, 20 minutes, 60 minutes, 2 hours, 3 hours, 4, hours, 8 hours, 12 hours, 24 hours of administration of the other. When the composition comprising cardiomyocytes and/or the SAP and/or the pharmaceutically active agent are administered in different pharmaceutical compositions, routes of administration can be different. In some embodiments, a subject is administered a composition comprising cardiomyocytes. In other embodiments, a subject is administered a composition comprising a pharmaceutically active agent. In other embodiments, a subject is administered a composition comprising a SAP. In another embodiment, a subject is administered a composition comprising a population of cardiomyocytes and a SAP mixed with a pharmaceutically active agent. In another embodiment, a subject is administered a composition comprising a population of cardiomyocytes and a SAP and a composition comprising a pharmaceutically active agent, where administration is substantially at the same time, or subsequent to each other. In another embodiment, a subject is administered a composition comprising a population of cardiomyocytes, a composition comprising a SAP, and a composition comprising a pharmaceutically active agent, where administration is substantially at the same time, or subsequent to each other.

Toxicity and therapeutic efficacy of administration of compositions comprising a population of cardiomyocytes can be determined by standard pharmaceutical procedures in cell cultures or experimental animals, e.g., for determining the LD50 (the dose lethal to 50% of the population) and the ED50 (the dose therapeutically effective in 50% of the population). Compositions comprising a population of cardiomyocytes that exhibit large therapeutic indices are preferred.

The amount of a composition comprising a population of cardiomyocytes can be tested using several well-established animal models.

In some embodiments, data obtained from the cell culture assays and in animal studies can be used in formulating a range of dosages for use in humans. The dosage of such compounds lies preferably within a range of circulating concentrations that include the ED50 with little or no toxicity. The dosage may vary within this range depending upon the dosage form employed and the route of administration utilized.

The therapeutically effective dose of a composition comprising a population of cardiomyocytes can also be estimated initially from cell culture assays. Alternatively, the effects of any particular dosage can be monitored by a suitable bioassay.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alterations to a treatment regimen. The dosing schedule can vary from once a week to daily depending on a number of clinical factors. The desired dose can be administered at one time or divided into subdoses, e.g., 2-4 subdoses and administered over a period of time, e.g., at appropriate intervals through the day or other appropriate schedules. Such sub-doses can be administered as unit dosage forms. In some embodiments, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

In another aspect of the invention, the methods provide use of an isolated population of cardiomyocytes as disclosed herein. In one embodiment of the invention, an isolated population of cardiomyocytes as disclosed herein may be used for the production of a pharmaceutical composition, for the use in transplantation into subjects in need of treatment, e.g. a subject that has, or is at risk of developing a ventricular arrhythmia or decreased systolic heart function (e.g., chronic heart failure). In one embodiment, an isolated population of cardiomyocytes may be genetically modified. In another aspect, the subject may have or be at risk of ventricular arrhythmias or decreased systolic heart function. In some embodiments, an isolated population of cardiomyocytes as disclosed herein may be autologous and/or allogeneic. In some embodiments, the subject is a mammal, and in other embodiments the mammal is a human.

One embodiment of the invention relates to a method of treating chronic heart failure in a subject comprising administering an effective amount of a composition comprising a population of cardiomyocytes as disclosed herein to a subject with chronic heart failure. Other embodiments relate to a method of treating a ventricular arrhythmia in a subject comprising administering an effective amount of a composition comprising a population of cardiomyocytes as disclosed herein to a subject with a ventricular arrhythmia. In a further embodiment, the invention provides a method for treating decreased systolic heart function, comprising administering a composition comprising a population of cardiomyocytes as disclosed herein to a subject with decreased systolic heart function. In another embodiment, the invention provides a method for treating congenital heart disease comprising administering an effective amount of a composition comprising a population of cardiomyocytes as disclosed herein to a subject with congenital heart disease.

In some embodiments, a population of cardiomyocytes as disclosed herein may be administered in any physiologically acceptable excipient, where the cardiomyocytes may find an appropriate site for replication, proliferation, and/or engraftment. In some embodiments, a population of cardiomyocytes as disclosed herein can be introduced by injection, catheter, or the like. In some embodiments, a population of cardiomyocytes as disclosed herein can be frozen at liquid nitrogen temperatures and stored for long periods of time, and is capable of use on thawing. If frozen, a population of cardiomyocytes will usually be stored in a 10% DMSO, 50% FCS, 40% RPMI 1640 medium or other cryoprotective solution. Once thawed, the cells may be expanded by use of growth factors and/or feeder cells associated with culturing cardiomyocytes as disclosed herein.

In some embodiments, a population of cardiomyocytes as disclosed herein can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition comprising a population of cardiomyocytes as disclosed herein will be adapted in accordance with the route and device used for administration. In some embodiments, a composition comprising a population of cardiomyocytes can also comprise or be accompanied with one or more other ingredients that facilitate the engraftment or functional mobilization of the cardiomyocytes. Suitable ingredients include matrix proteins that support or promote adhesion of the cardiomyocytes, or complementary cell types. In another embodiment, the composition may comprise resorbable or biodegradable matrix scaffolds.

Gene therapy can be used to either modify a cell to replace a gene product, to facilitate regeneration of tissue, to treat disease, or to improve survival of the cells following implantation into a subject (i.e., prevent rejection).

In one aspect of the present invention, a population of cardiomyocytes and/or a SAP as disclosed herein is suitable for administering systemically or to a target anatomical site. A population of cardiomyocytes, optionally with a SAP, can be grafted into or nearby a subject's heart, for example, or may be administered systemically, such as, but not limited to, intra-arterial or intravenous administration. In alternative embodiments, a population of cardiomyocytes, optionally with a SAP, can be administered in various ways as would be appropriate to implant in the cardiac system, including but not limited to parenteral, including intravenous and intraarterial administration, intrathecal administration, intraventricular administration, intraparenchymal, intracranial, intracisternal, intrastriatal, and intranigral administration. Optionally, a population of cardiomyocytes, optionally with a SAP, is administered in conjunction with an immunosuppressive agent.

In some embodiments, a population of cardiomyocytes, optionally with a SAP, can be administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement, including but not limited to improved survival rate or more rapid recovery, or improvement or elimination of symptoms and other indicators as are selected as appropriate measures by those skilled in the art. A population of cardiomyocytes can be administered to a subject at the following locations: clinic, clinical office, emergency department, hospital ward, intensive care unit, operating room, catheterization suites, and radiologic suites.

In other embodiments, a population of cardiomyocytes is stored for later implantation/infusion. A population of cardiomyocytes may be divided into more than one aliquot or unit such that part of a population of cardiomyocytes is retained for later application while part is applied immediately to the subject. Moderate to long-term storage of all or part of the cells in a cell bank is also within the scope of this invention, as disclosed in U.S. Patent Publication No. 2003/0054331 and Patent Publication No. WO 03/024215, and are incorporated by reference in their entireties. At the end of processing, the concentrated cells may be loaded into a delivery device, such as a syringe, for placement into the recipient by any means known to one of ordinary skill in the art.

In some embodiments a population of cardiomyocytes can be applied alone or in combination with other cells, tissue, tissue fragments, growth factors such as VEGF and other known angiogenic or arteriogenic growth factors, biologically active or inert compounds, resorbable plastic scaffolds, or other additives intended to enhance the delivery, efficacy, tolerability, or function of the population. In some embodiments, a population of cardiomyocytes may also be modified by insertion of DNA or by placement in cell culture in such a way as to change, enhance, or supplement the function of the cells for derivation of a structural or therapeutic purpose. For example, gene transfer techniques for stem cells are known by persons of ordinary skill in the art, as disclosed in (Morizono et al., 2003; Mosca et al., 2000), and may include viral transfection techniques, and more specifically, adeno-associated virus gene transfer techniques, as disclosed in (Walther and Stein, 2000) and (Athanasopoulos et al., 2000). Non-viral based techniques may also be performed as disclosed in (Murarnatsu et al., 1998).

In another aspect, in some embodiments, a population of cardiomyocytes could be combined with a gene encoding pro-angiogenic growth factor(s). Genes encoding anti-apoptotic factors or agents could also be applied. Addition of the gene (or combination of genes) could be by any technology known in the art including but not limited to adenoviral transduction, “gene guns,” liposome-mediated transduction, and retrovirus or lentivirus-mediated transduction, plasmid adeno-associated virus. Cells could be implanted along with a carrier material bearing gene delivery vehicle capable of releasing and/or presenting genes to the cells over time such that transduction can continue or be initiated. Particularly when the cells and/or tissue containing the cells are administered to a patient other than the patient from whom the cells and/or tissue were obtained, one or more immunosuppressive agents may be administered to the patient receiving the cells and/or tissue to reduce, and preferably prevent, rejection of the transplant. As used herein, the term “immunosuppressive drug or agent” is intended to include pharmaceutical agents which inhibit or interfere with normal immune function. Examples of immunosuppressive agents suitable with the methods disclosed herein include agents that inhibit T-cell/B-cell costimulation pathways, such as agents that interfere with the coupling of T-cells and B-cells via the CTLA4 and B7 pathways, as disclosed in U.S. Patent Publication No 2002/0182211, which is incorporated herein by reference. In one embodiment, an immunosuppressive agent is cyclosporine A. Other examples include myophenylate mofetil, rapamicin, and anti-thymocyte globulin. In one embodiment, the immunosuppressive drug is administered with at least one other therapeutic agent. The immunosuppressive drug is administered in a formulation which is compatible with the route of administration and is administered to a subject at a dosage sufficient to achieve the desired therapeutic effect. In another embodiment, the immunosuppressive drug is administered transiently for a sufficient time to induce tolerance to the cardiomyocytes of the invention.

Pharmaceutical compositions comprising effective amounts of a population of cardiomyocytes are also contemplated by the present invention. These compositions comprise an effective number of cardiomyocytes, optionally, in combination with a pharmaceutically acceptable carrier, additive or excipient and optionally with a self-assembling peptide (SAP). In certain aspects of the present invention, a population of cardiomyocytes is administered to the subject in need of a transplant in sterile saline. In other aspects of the present invention, a population of cardiomyocytes is administered in Hanks Balanced Salt Solution (HBSS) or Isolyte S, pH 7.4. Other approaches may also be used, including the use of serum free cellular media. In one embodiment, a population of cardiomyocytes is administered in plasma or fetal bovine serum, and DMSO. Systemic administration of a population of cardiomyocytes to the subject may be preferred in certain indications, whereas direct administration at the site of or in proximity to the diseased and/or damaged tissue may be preferred in other indications.

In some embodiments, a population of cardiomyocytes can optionally be packaged in a suitable container with written instructions for a desired purpose, such as the reconstitution or thawing (if frozen) of a population of cardiomyocytes prior to administration to a subject.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the disclosure. Further, all patents, patent applications, and publications identified are expressly incorporated herein by reference for the purpose of describing and disclosing, for example, the methodologies described in such publications that might be used in connection with the disclosure. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents

### EXAMPLES

### Example 1: Flexible Nanoelectronics Reveal Arrhythmogenesis in Transplanted Human Cardiomyocytes

Cardiac tissue engineering has emerged as a promising strategy for repairing damaged myocardium, particularly through the use of human induced pluripotent stem cell-derived cardiomyocytes (hiPSC-CMs) (1). However, despite significant advances, differentiated hiPSC-CMs generated by current methods often exhibit underdeveloped phenotypes characterized by fetal-like sarcomere organization, insufficient expression of adult myosin-heavy chains, and a propensity for arrhythmogenic automaticity (2, 3). hiPSC-CMs transplanted into cardiac tissue can exhibit increased automatic firing, or arrhythmogenic automaticity; this is thought to be the origin of potentially lethal ventricular arrhythmias observed in large animal models (4, 5). Murry and colleagues showed that targeting automaticity with CRISPR/Cas9 gene editing of multiple genes can reduce ventricular arrhythmias (6). Establishing safe, efficacious hiPSC-CM transplantation technologies for human use remains an important translational goal.

Previous studies have shown that injectable biomaterials can improve the cellular microenvironment and increase the efficiency of cell therapy (7, 8). RADA16, a self-assembling peptide approved for clinical use, has shown the potential to improve cellular retention, organization, and vascularization in tissue-engineering applications (9, 10). Here, the inventors studied self-assembling peptides as injectable microenvironments to evaluate their impact on the in vivo adaptation of transplanted hiPSC-CMs using a rat model. To assess electrophysiological integration, the inventors employed advanced flexible mesh nanoelectronics to capture cellular resolution voltage signals at both injection and reference sites. These recordings provided unprecedented insights into electrical signal propagation and automaticity over months after transplantation. A study demonstrates that co-injection with RADA16 mitigates arrhythmogenic automaticity, promotes vascularization, and supports adult-like sarcomere development, highlighting its potential to overcome key barriers to hiPSC-CMs transplantation and cardiac regeneration.

### Results

### Gene Expression Analysis of Cardiomyocytes Cultured on Self-Assembling Peptides

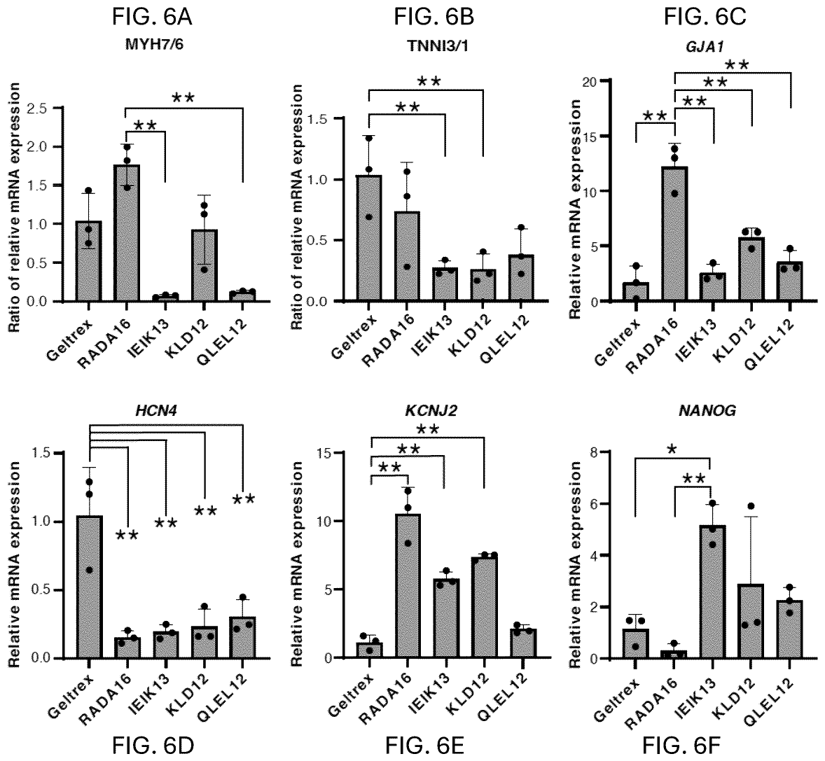

Quantitative polymerase chain reaction (qPCR) results revealed that gene expression profiles of hiPSC-CMs differed when cultured on various self-assembling peptides (SAPs) and Geltrex (control group), exhibiting differences in the expression of key genes related to myofibril development (FIG. 6). The ratio of MYH7 mRNA expression to MYH6 expression was significantly higher in the RADA16 group than in the IEIK13 and QLEL12 groups; this ratio is an indicator of the myosin-heavy chain switching from a human fetal isoform to an adult isoform (11). However, the ratio was not significantly different between the RADA16 and control groups nor the RADA16 and KLD12 groups (FIG. 6A). A one-way ANOVA post hoc Tukey test showed no significant difference in the ratio of TNNI3 to TNNI1 expression in all SAP groups (FIG. 6B). The expression of GJA1, the gap junction coding gene, was significantly higher in the RADA16 group compared to all other SAP groups and the control group (FIG. 6C). Next, HCN4, which encodes an ion channel protein prominently found in pacemaker cells, showed significantly decreased expression in the SAP groups as compared to the control group (FIG. 6D). Further, expression of KCNJ2, the potassium ion channel coding gene, was significantly higher in the RADA16, IEIK13, and KLD12 groups compared to the control group (FIG. 6E). The expression of NANOG, an indicator of pluripotency, was significantly lower in the RADA16 and control groups than in the IEIK13 group, but there was no significant difference between the control and RADA16 group (FIG. 6F). Altogether, analysis of cardiomyocyte gene expression when cultured on different SAPs pointed to RADA16 as a promising factor for promoting adult-like transcriptomic profiles. Thus, the inventors proceeded to in vivo testing with RADA16, since this self-assembling peptide is already approved for human use (9, 12, 13).

Integration and Vascularization of Injectable Cardiomyocytes within the Rats' Heart

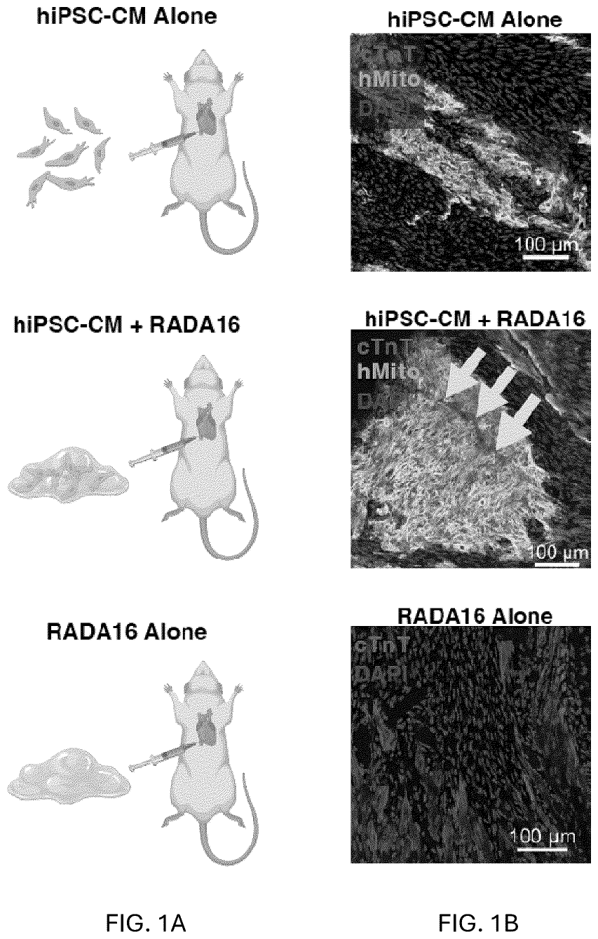

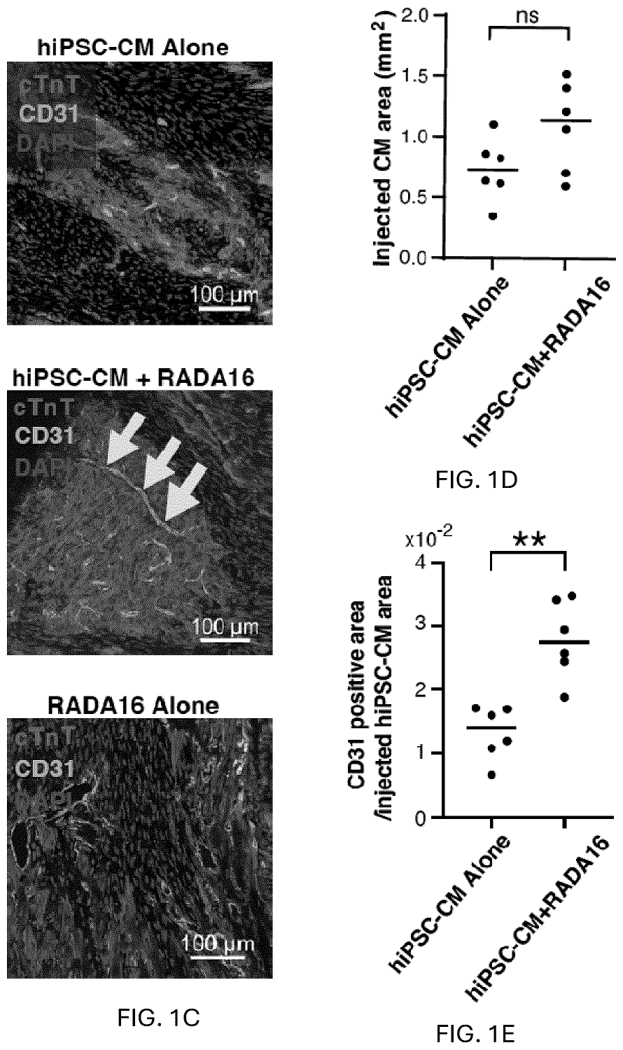

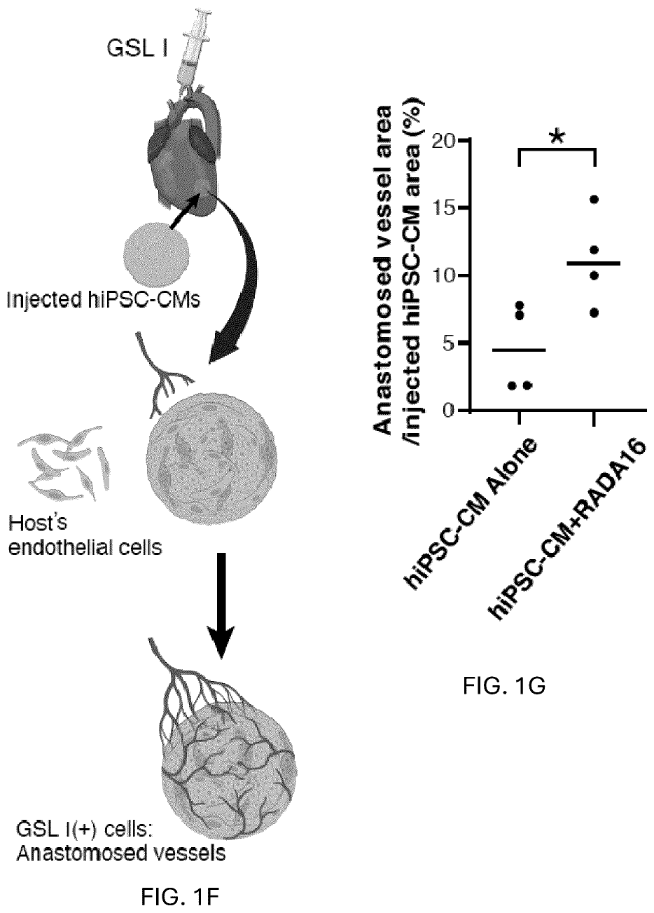

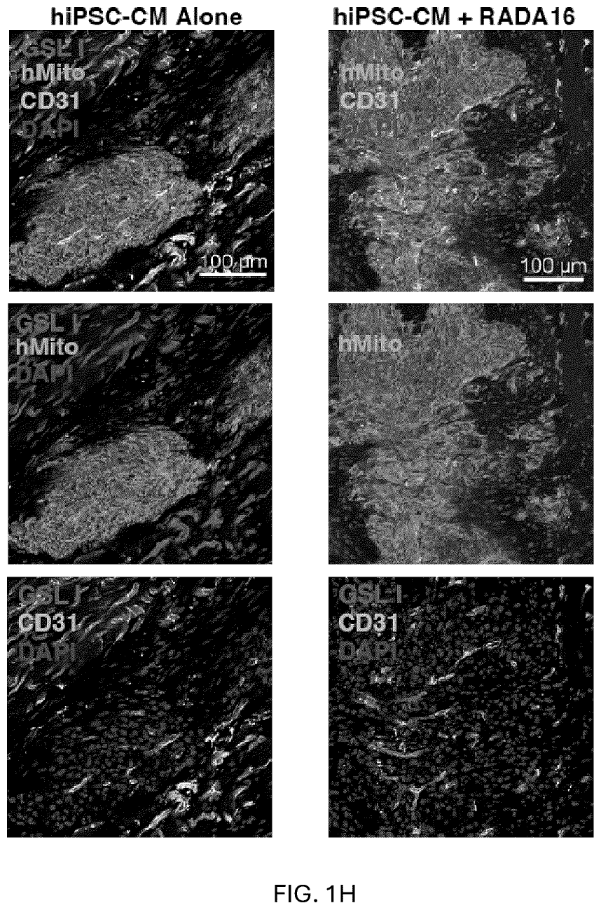

Immunofluorescence staining of sliced sections of the rat heart revealed that both the injected hiPSC-CMs solution (hiPSC-CM Alone) and the injected hiPSC-CMs with RADA16 solution (hiPSC-CM+RADA16) were engrafted and vascularized within the rat heart tissue at day 7 (FIGS. 1B and 1C). The inventors confirmed engraftment of hiPSC-CMs by detecting hiPSC-CMs area positive for both anti-human mitochondrial antibody (hMito) and anti-cardiac troponin T antibody (cTnT) in the hiPSC-CM Alone and hiPSC-CM+RADA16 experimental groups; as expected, the group with only RADA16 injected (no hiPSC-CMs, negative control: RADA16 Alone) lacked area positive for hMito (FIG. 1B). Also, vessel-like structures were positive for anti-CD31 antibody (CD31) in each injection group (FIG. 1C). Although not a statistically significant difference, the injected hiPSC-CMs area in the hiPSC-CM+RADA16 group tended to be greater than for the hiPSC-CM Alone injection group (FIG. 1D). However, the CD31-positive vessels were negative for hMito in each group, (FIGS. 1B and 1C), indicating that vascularization was driven by the formation of host blood vessels. Importantly, the ratio of vascularization area (CD31 positive area) inside the injected hiPSC-CMs sites (hMito positive area) was significantly higher in the hiPSC-CM+RADA16 group than in the hiPSC-CM Alone group (FIG. 1E) in 6 each rat model.

To determine if RADA16-promoted vessels functionally anastomosed with host vessels within the injected hiPSC-CMs area, the inventors injected GSL I (Griffonia Simplicifolia Lectin I Isolectin B4, DyLight 649 (DL-1208-. 5)) into the coronary arteries directly to visualize rat vascular endothelial cells and anastomosed neovessels within the transplanted hiPSC-CMs (FIGS. 1F and 1H). The inventors then analyzed the interaction of the GSL I- and CD31-positive area, which indicates the presence of vessels. Most of the vessels within the hiPSC-CMs were positive for both CD31 and GSL I (FIG. 1H). The proportion of GSL I-positive vessel area within the hiPSC-CMs area (positive for hMito) was significantly higher in the hiPSC-CM+RADA16 group compared to the hiPSC-CM Alone group on 4 each rat samples (FIG. 1G). Thus, the neovessels within the hiPSC-CMs site were functionally anastomosed to the host vasculature.

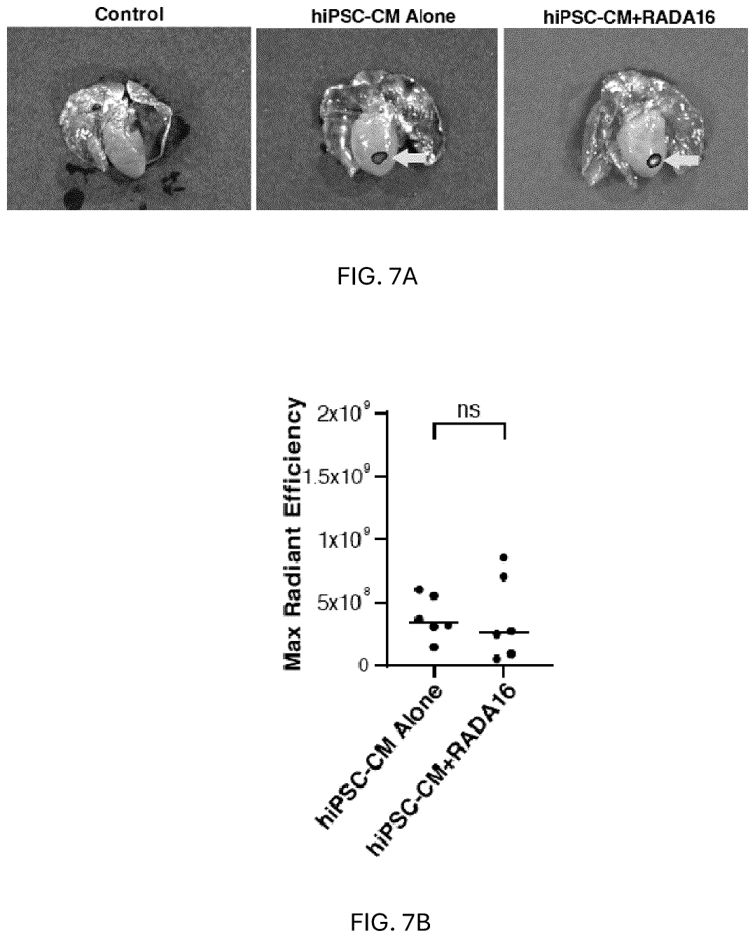

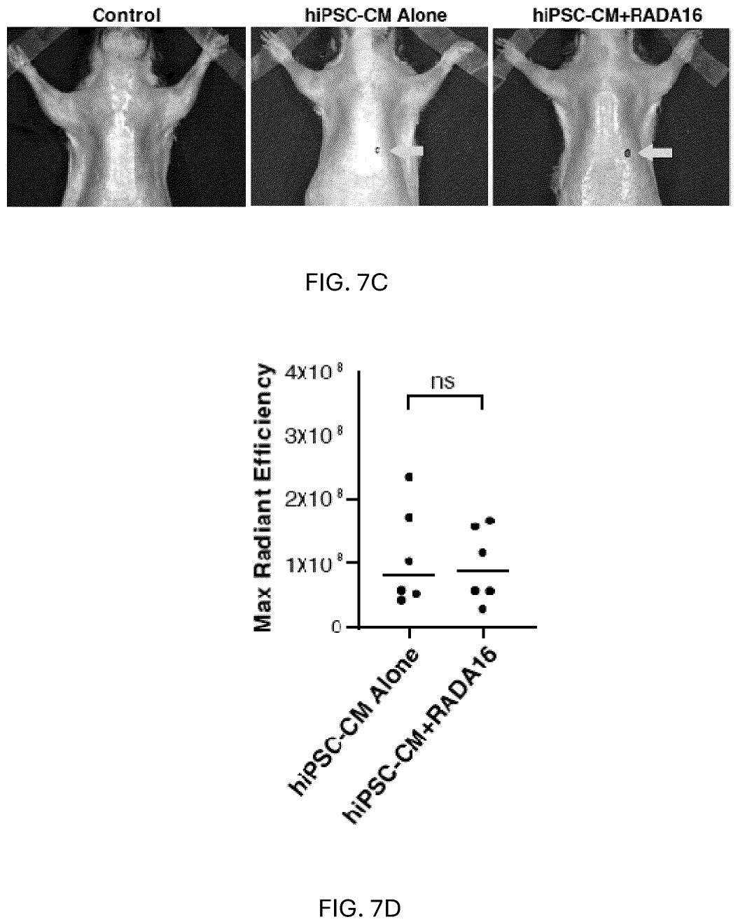

Previous reports have suggested that injectable hydrogels can increase cell retention following injection (14, 15). To determine if RADA16 increased the retention of transplanted hiPSC-CMs, the inventors utilized an in vivo imaging system (IVIS) to observe the bioluminescence on the surface of the left ventricular wall of the rats' hearts with ex vivo samples (FIG. 7A) and within the rat body surface with in vivo samples (FIG. 7C). For both ex vivo and in vivo samples across 6 different rat models, there were no significant differences in the Max Radiant Efficiency (which represents labeled cell retention) between the hiPSC-CM Alone and hiPSC-CM+RADA16 groups (FIGS. 7B and 7D). This suggested RADA16 co-injection with iPS-derived CMs does not necessarily improve cell retention.

Spatial Genomics Shows that RADA16 Promotes Adult-Like Gene Expression for hiPSC-CMs in Rat Hearts.

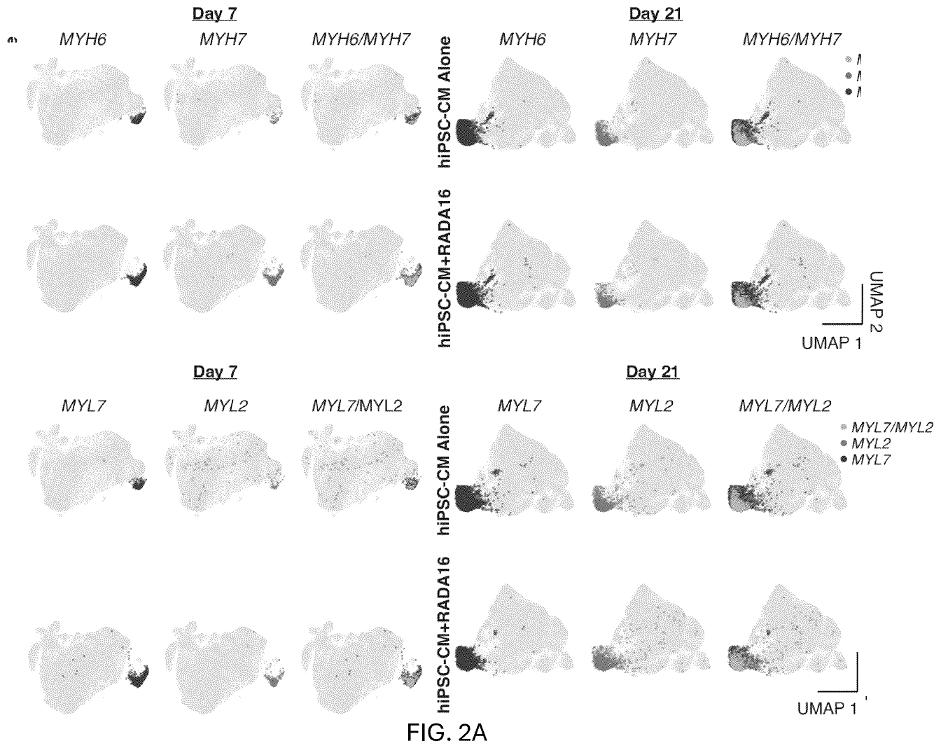

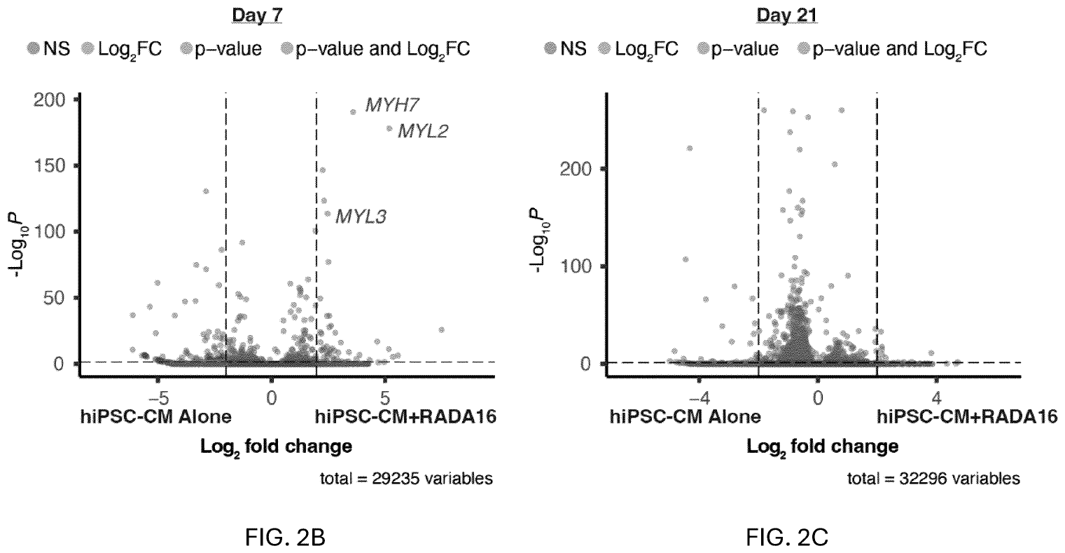

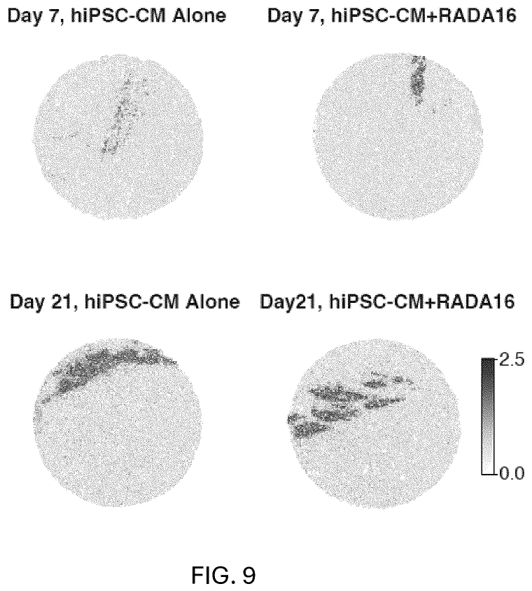

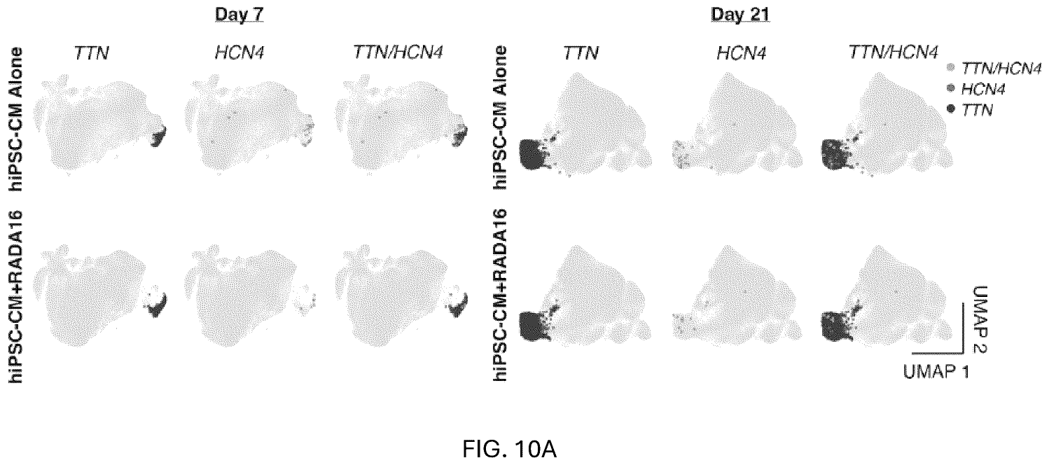

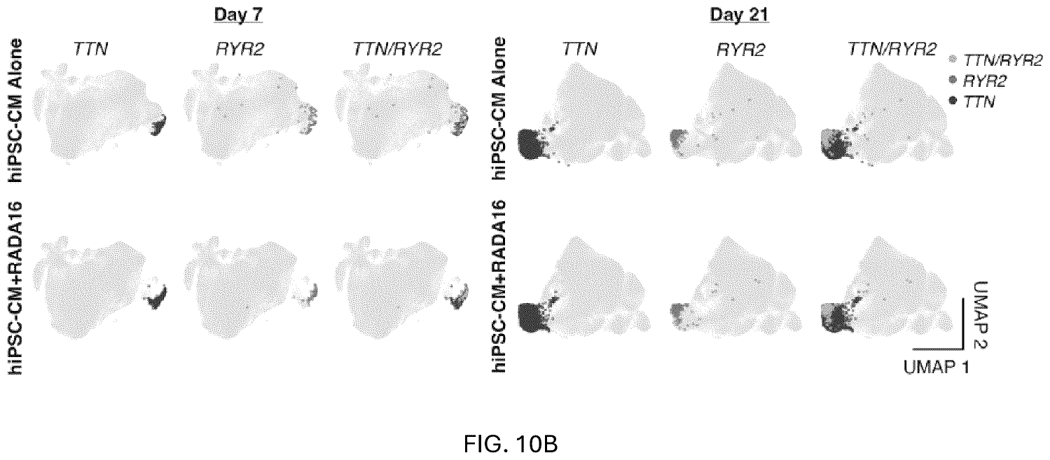

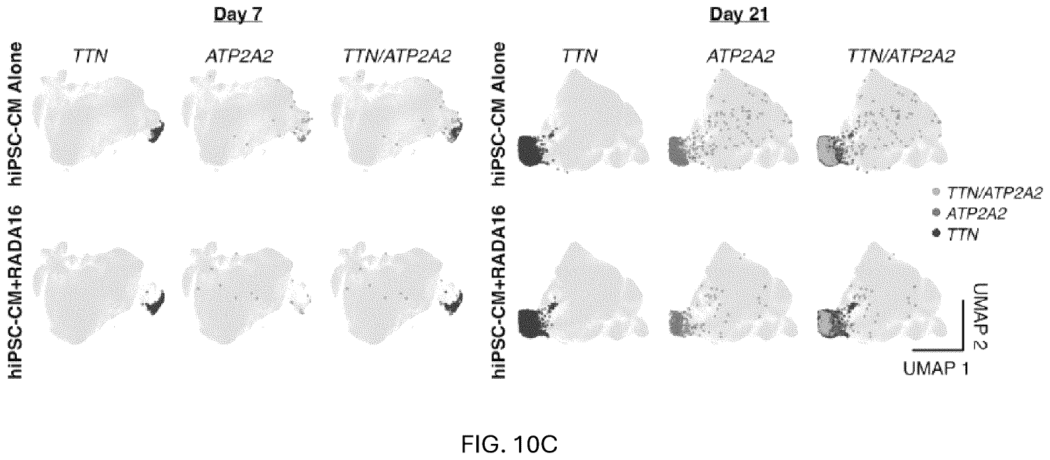

The Slide-seq technique was applied to study gene expression switches induced by RADA16 in hiPSC-CMs transplanted to rat myocardium (16). Spatial feature plots detected the spatial localization of hiPSC-CMs (as assessed by TTN expression). The inventors observed that in both hiPSC-CM Alone and hiPSC-CM+RADA16 samples, there are instances of clear localization of the human cells (FIG. 9). In assessments of transcriptomic markers of cardiomyocyte development in the Slide-seq data, sparse expression of ion channel genes was observed when comparing treatments (FIG. 10), making it difficult to draw any conclusions regarding electrophysiological characteristics from Slide-seq alone. At day 7, there were marked shifts in sarcomeric isoforms towards adult-like isoforms in the hiPSC-CM+RADA16 group compared to the hiPSC-CM Alone group (FIG. 2A).

Differential gene expression analysis on hiPSC-CMs (subsetted by human TNNT2 expression) showed that at the day 7 timepoint, expression of MYH7 and MYL2 is significantly increased in the hiPSC-CM+RADA16 group relative to the hiPSC-CM Alone group. These genes, however, were no longer differentially expressed in the day 21 hearts (FIGS. 2B and 2C). This suggests that this difference at day 7 is the result of more rapid sarcomeric adaptation from RADA16 treatment, instead of a greater overall degree of adaptation. The full list of differentially expressed genes is provided in Table S1.

RADA16 Promotes Sarcomere Organization, and Vascularization of Injected hiPSC-CMs in Rat Hearts Over Longer Periods

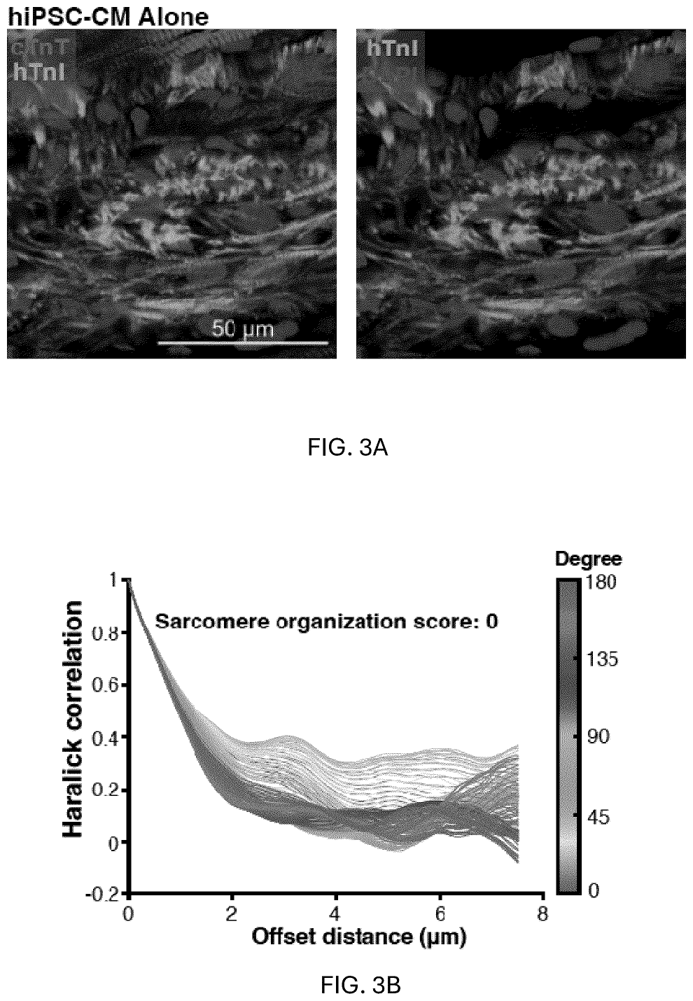

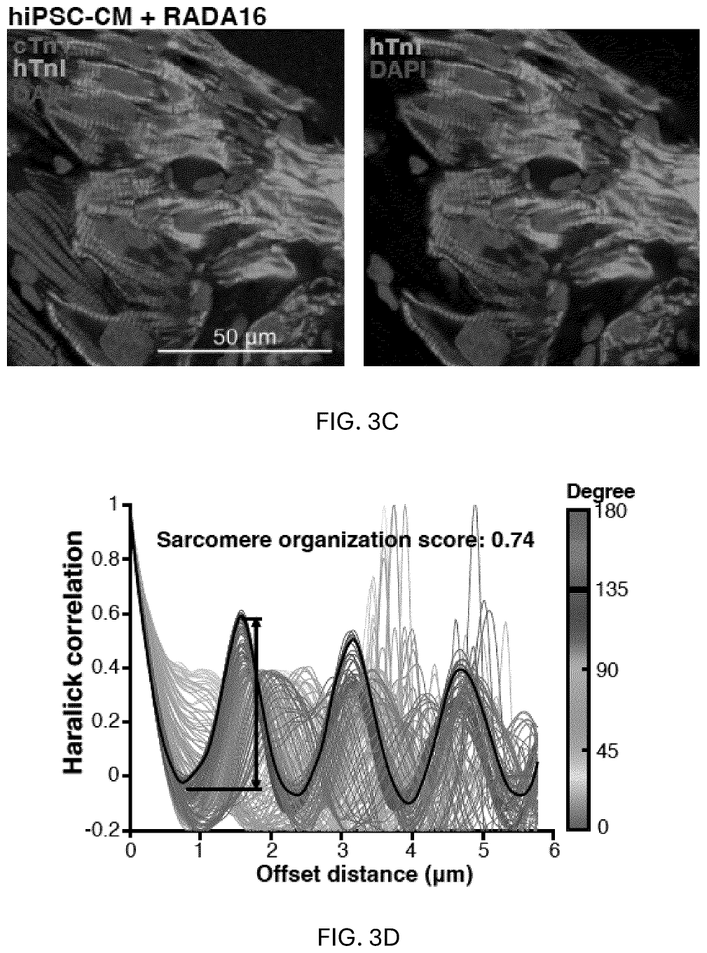

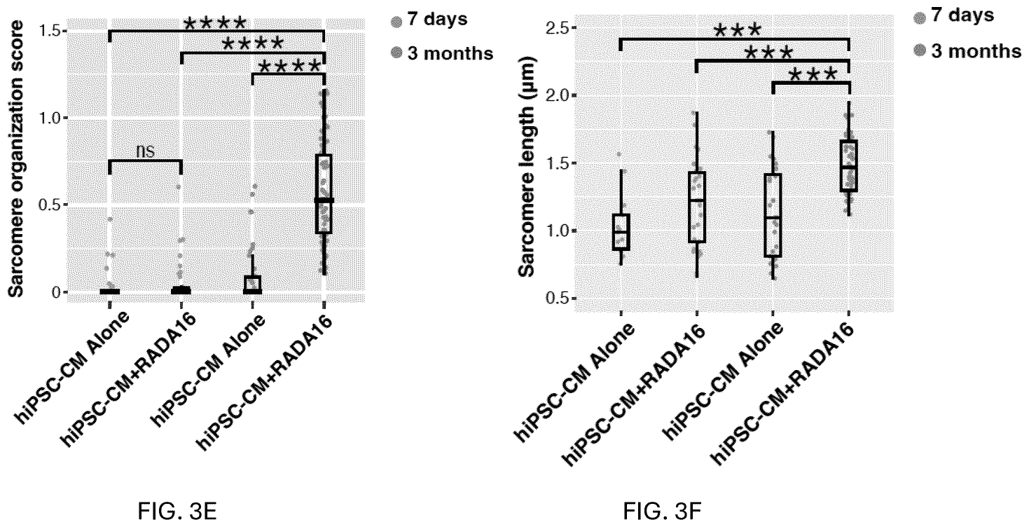

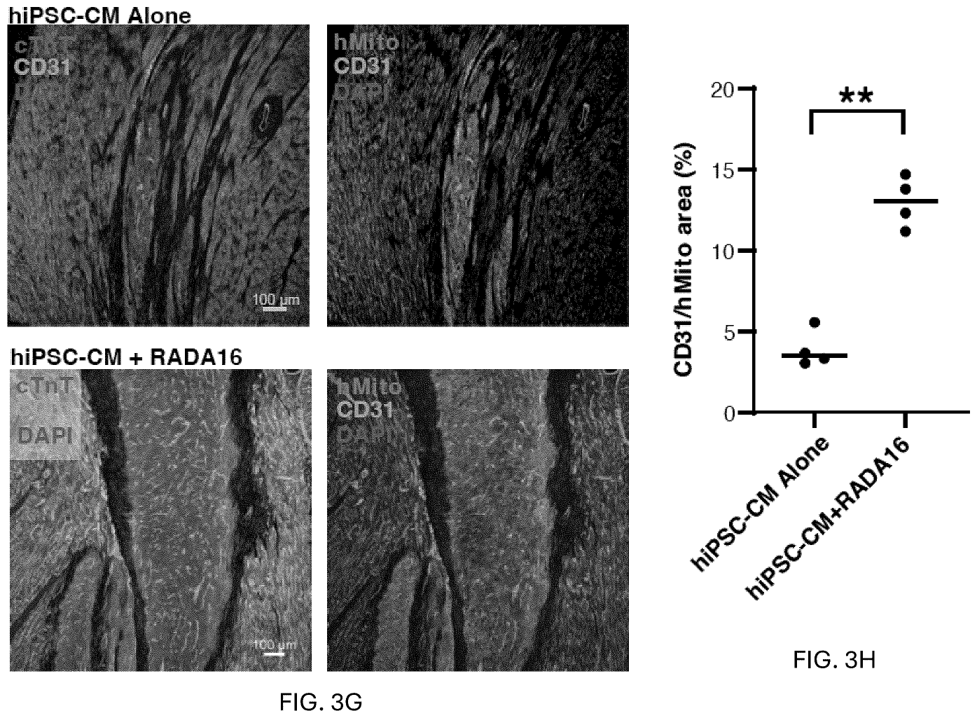

The inventors hypothesized that the accelerated pattern of the adult-like gene expression due to RADA16 may coincide with improved sarcomere adaptation of injected hiPSC-CMs. To assess whether RADA16 promotes sarcomere organization of co-injected hiPSC-CMs, the inventors performed immunofluorescence staining for 3 months. Both hiPSC-CM Alone and hiPSC-CM+RADA16 exhibited minimal sarcomere organization 7 days after injection in the rat's left ventricle (17). However, by 3 months, the hiPSC-CM+RADA16 exhibited much greater sarcomere organization (FIGS. 3A and 3C). To provide a quantitative measure of sarcomere organization, the inventors applied a previously validated analysis, SarcOmere Texture Analysis (SOTA), on myofibril bundles from across all tissue samples following systemic injection at 7 days and 3 months post-injection (17). SOTA Haralick correlation showed that the hiPSC-CM+RADA16 exhibited highly structured patterns consistent with well-organized sarcomeres, whereas the hiPSC-CM Alone group did not (FIGS. 3B and 3D). SOTA revealed a marked increase in the sarcomere organization of the hiPSC-CM+RADA16 group compared to the hiPSC-CM Alone group at 3 months and compared to the hiPSC-CM+RADA16 at 7 days (FIG. 3E). This demonstrates a significant increase in sarcomere development in the hiPSC-CM+RADA16 between 7 days and 3 months.

As an additional measure of sarcomere adaptation in vivo, the inventors computed the intercellular alignment of sarcomere orientation via the difference from the mean orientation from SOTA Haralick correlation plots. There was a significant difference in variances across all groups. The hiPSC-CM+RADA16 at 3 months showed the greatest intercellular alignment of sarcomere orientation, i.e., the lowest standard deviations of orientation values (Table S2).

Because increased sarcomere length is another morphological hallmark of adult-like cardiomyocytes with developed morphological properties (18), the inventors measured sarcomere length of randomly selected myofibril bundles of hiPSC-CMs. While RADA16 was not associated with a difference in sarcomere length 7 days post-injection, at 3 months, the hiPSC-CM+RADA16 group had longer sarcomere lengths (FIG. 3F). Overall, measures of sarcomere organization by SOTA and sarcomere length demonstrated that by 3 months, RADA16 promoted in vivo development and characteristics indicative of adult-like cardiomyocytes.

Rats injected with hiPSC-CMs with and without RADA16 were held for 3 months, after which the inventors prepared cryosections of the hearts for immunofluorescent staining. Analysis with confocal microscopy revealed that the injected hiPSC-CMs (both the group with and the group without RADA16) were positive for anti-human mitochondrial antibody (hMito), and the hiPSC-CM injection area was vascularized with CD31-positive vessels (FIG. 3G). The proportion of vascularization area (positive for anti-CD31 antibody) within the area of injected hiPSC-CMs (positive for hMito) was significantly higher in the hiPSC-CM+RADA16 group compared to the hiPSC-CM Alone group (n=4) (FIG. 3H).

Electrophysiological Adaptation of hiPSC-CMs Induced by RADA16: Electrical Propagation and Decreased Arrhythmogenic Automaticity

To determine if RADA16 changes automaticity of hiPSC-CMs after transplantation into rat hearts, the inventors employed flexible electronics to enable high-resolution electrophysiological recording to differentiate hiPSC-CM signals from native cardiac signals. This approach is crucial for assessing the electrical function of hiPSC-CMs in the engineered heart tissues. Traditional methods, such as calcium imaging or conventional electrodes, often lack the spatiotemporal resolution needed to distinguish hiPSC-CM signals from host tissue signals and fail to maintain high spatiotemporal resolution mapping in dynamically contracting hearts.

Recent advances in flexible nanoelectronics have enabled highly sensitive and spatially resolved electrical mapping of hiPSC-CMs, and the flexibility allows deformation of the nanoelectronics with each heartbeat. For example, stretchable mesh nanoelectronics have been used to track and differentiate the millisecond-resolved electrical activities of hiPSC-CM organoids with cellular resolution (19). Here, the inventors applied flexible mesh nanoelectronics to record electrical signals from hiPSC-CMs transplanted into rat hearts, aiming to differentiate arrhythmogenic automaticity and sinus rhythm, assessing the propagation of arrhythmogenic automaticity and sinus rhythm signals and the impact of RADA16 on the electrophysiological properties of engineered cardiac tissues.

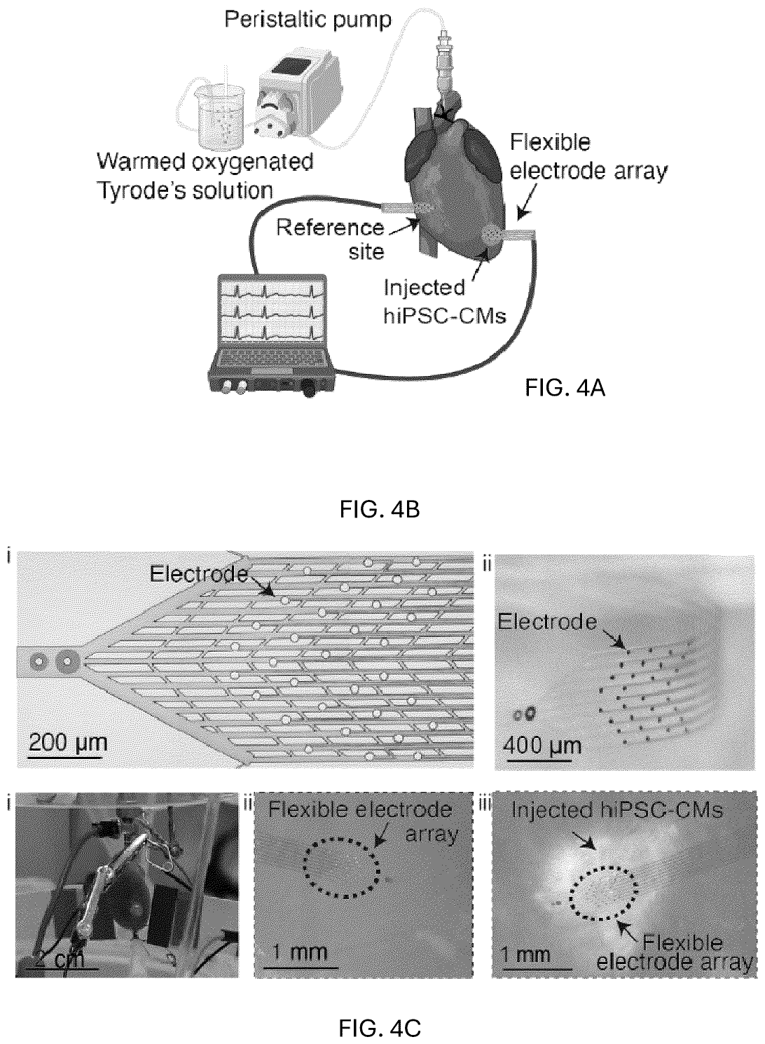

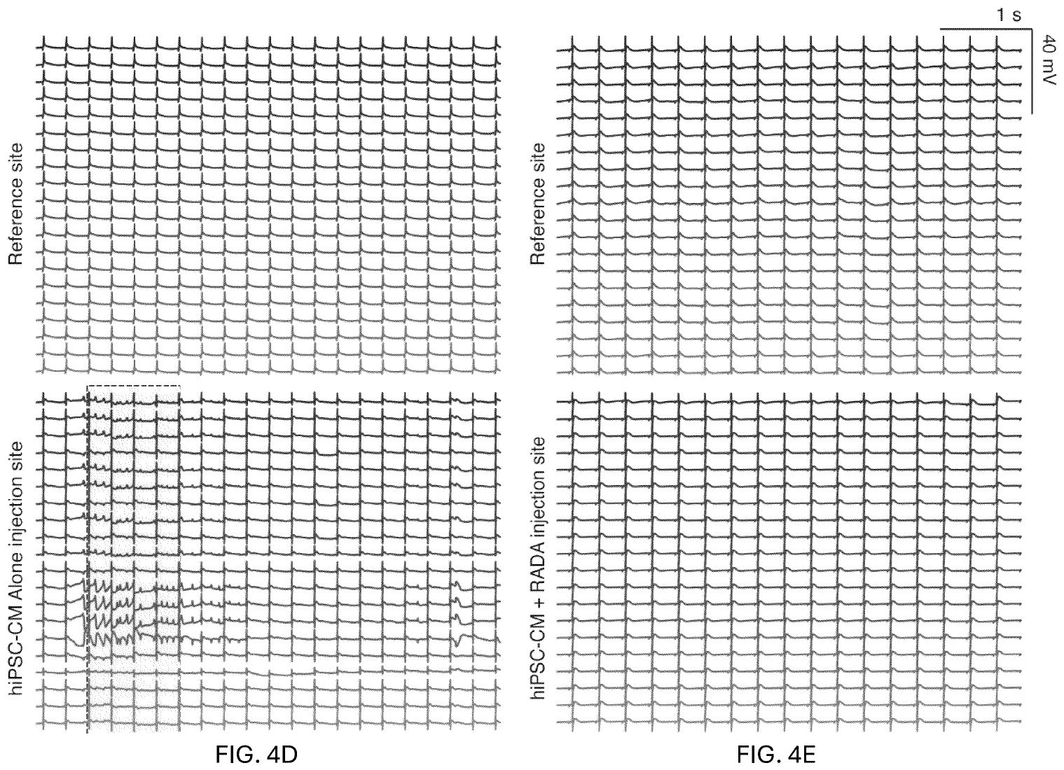

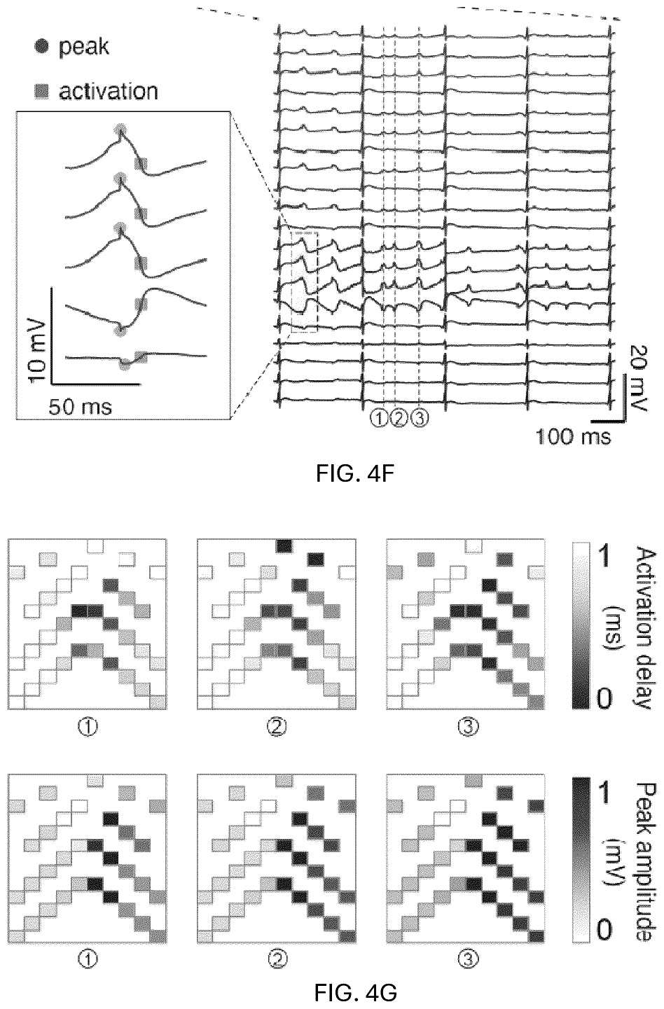

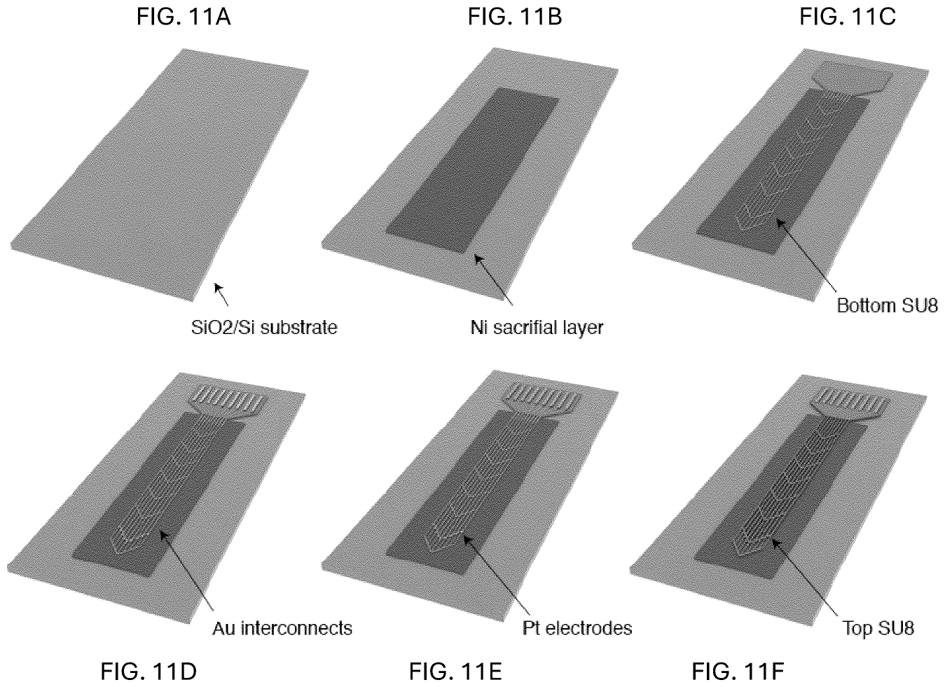

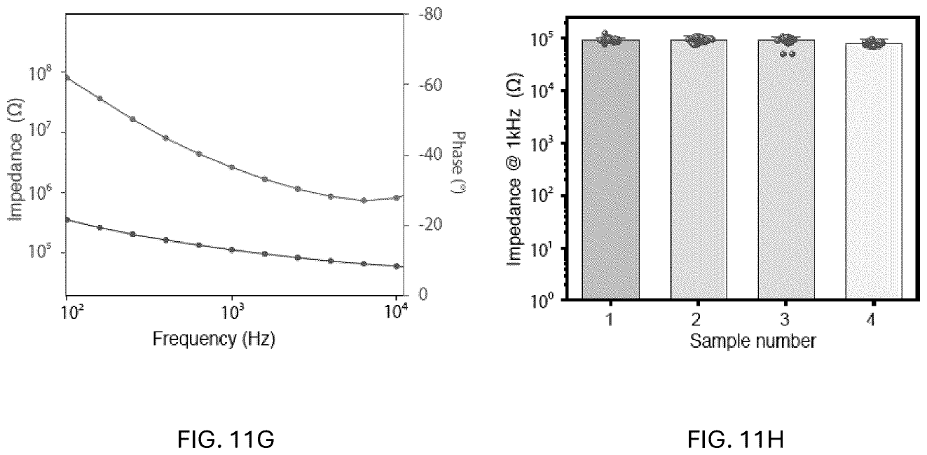

Firstly, the inventors tested rat hearts injected with hiPSC-CMs alone (hiPSC-CM Alone) or hiPSC-CMs combined with RADA16 (hiPSC-CM+RADA). Electrical signals were recorded simultaneously from both injection sites and non-injection reference sites of hearts for, with the latter serving as a reference for comparison. A custom-designed mesh nanoelectronics system with a high-density microelectrode array was employed (FIG. 4A). These devices were fabricated using previously established methods (FIG. 4B (i) and FIGS. 11A-11F). The microelectrodes were coated with Pt black, ensuring low and consistent impedance across samples (FIGS. 11G-11H). Upon release from the wafer, the devices became highly flexible, forming free-standing structures suitable for attachment to the heart for recording (FIG. 4B (ii)).

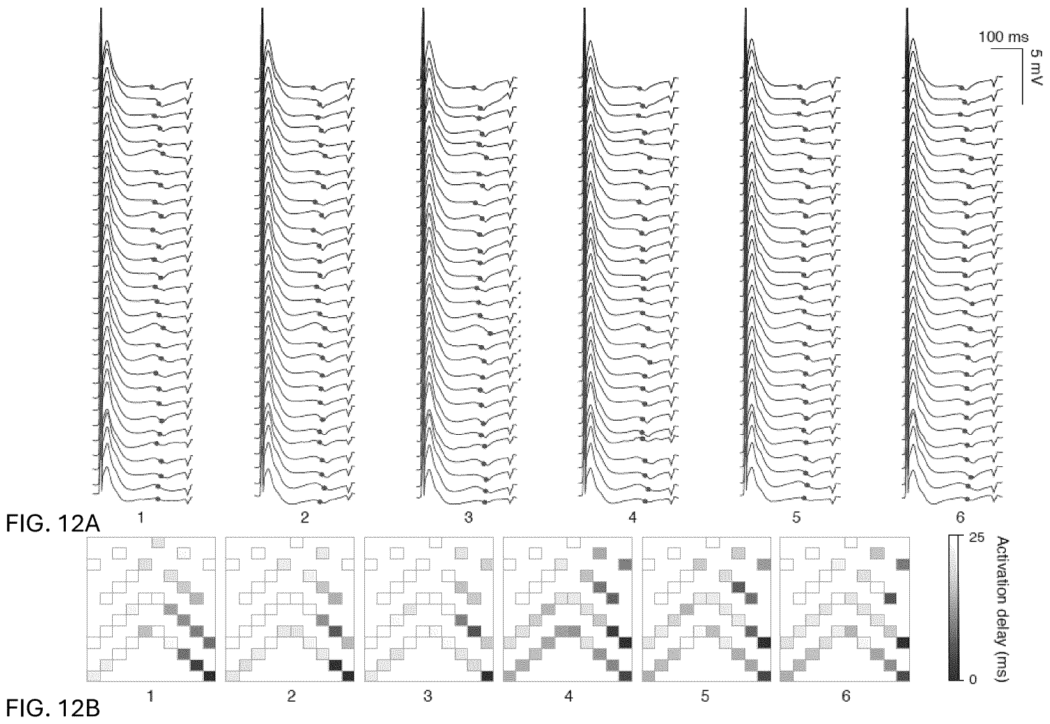

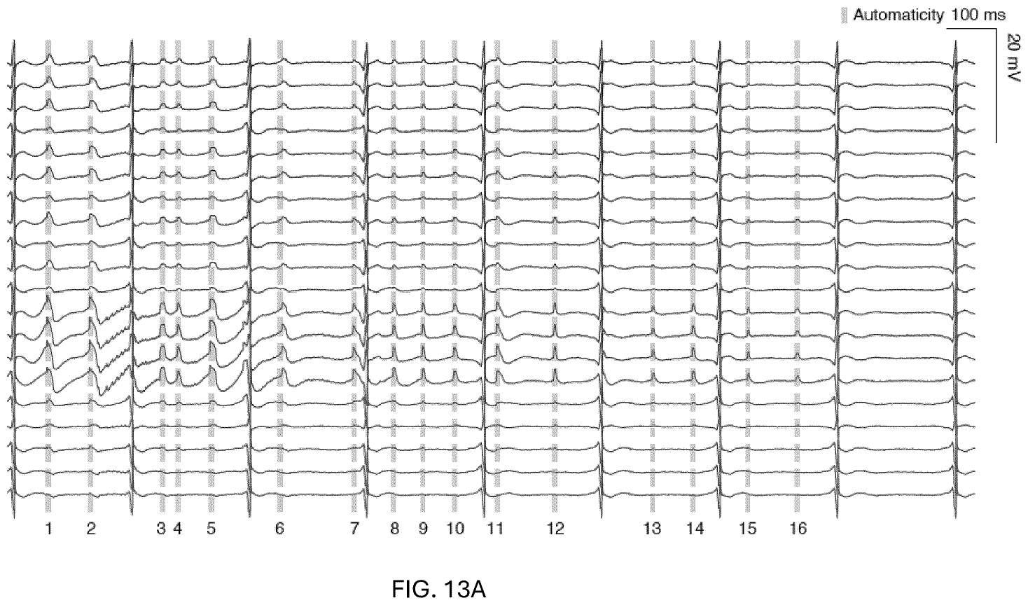

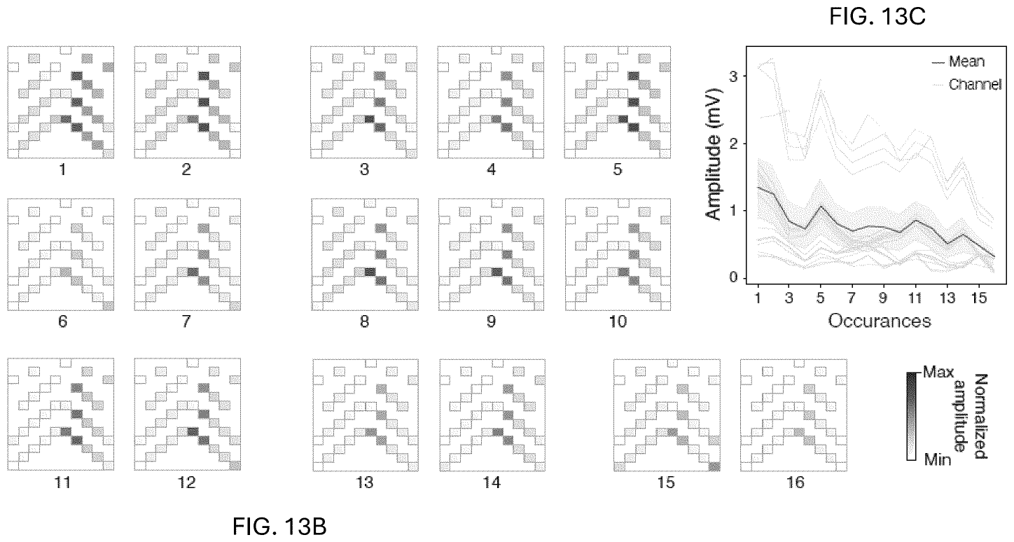

Following 80 days of post-transplantation, rat hearts were perfused with warmed, oxygenated Tyrode's solution via the Langendorff heart perfusion system. Two flexible mesh electrode arrays, each with 32 channels, (FIG. 4B) were attached to the reference and injection sites (hiPSC-CMs Alone or hiPSC-CM+RADA), respectively (FIG. 4C (i)). The inventors used bright-field (BF) and optical microscopy to confirm the proper positioning of the devices on the reference and injection sites (FIG. 4C (ii-iii)). Raw voltage traces recorded from both reference and hiPSC-CM Alone-injection sites (FIG. 4D) showed sinus rhythm. However, voltage traces from hiPSC-CM Alone-injection sites showed arrhythmogenic automaticity rhythms, as indicated by lower amplitudes, longer duration of the spike, and slower dynamics comparing to the native sinus rhythm. In contrast, no arrhythmogenic automaticity rhythms were detected at hiPSC-CM+RADA injection sites (FIG. 4E). Spatial mapping of arrhythmogenic automaticity spike peaks and propagation delays to corresponding physical locations of electrodes on the devices (FIGS. 4F-4G, FIGS. 12-13) revealed consistent radial propagation patterns at injection sites.

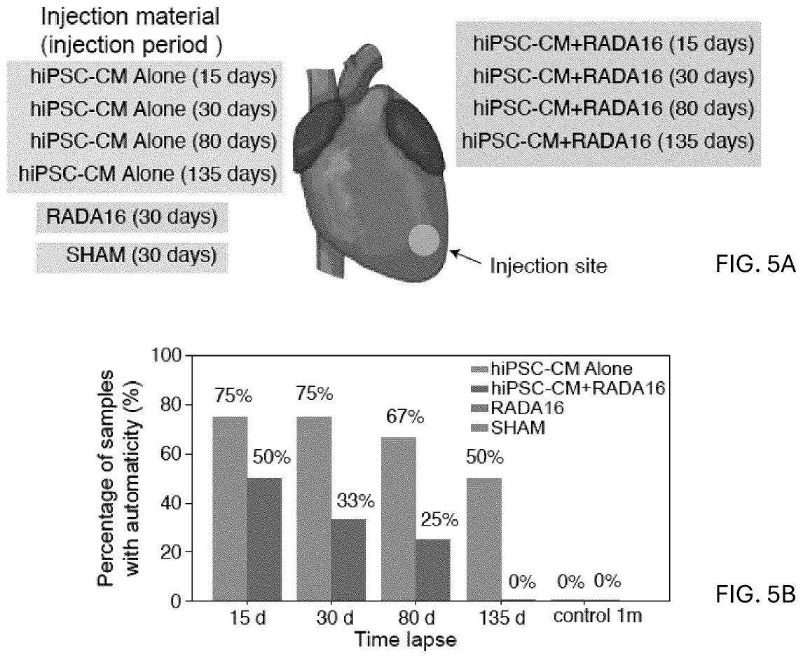

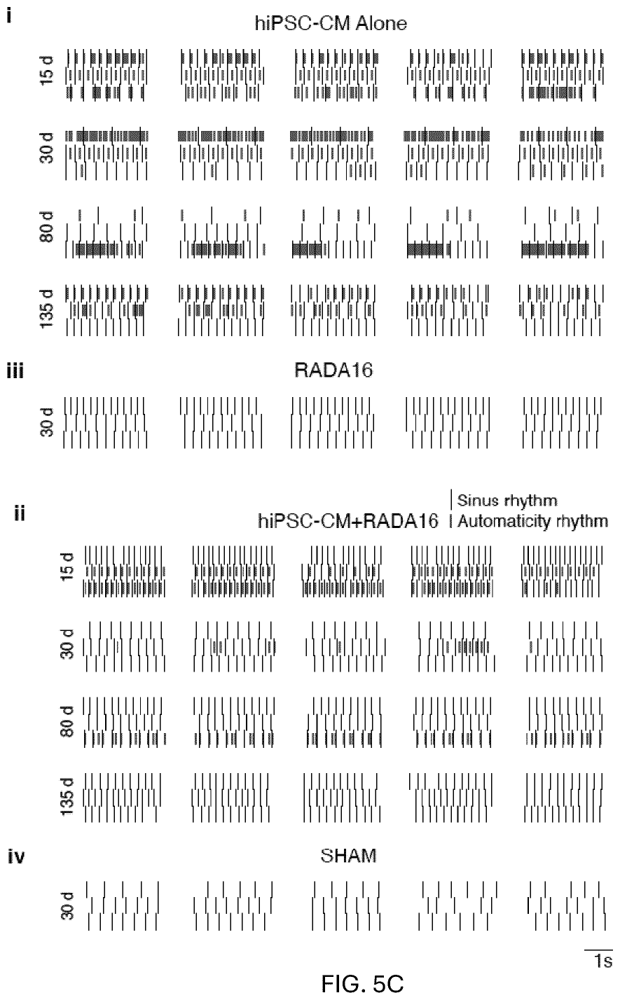

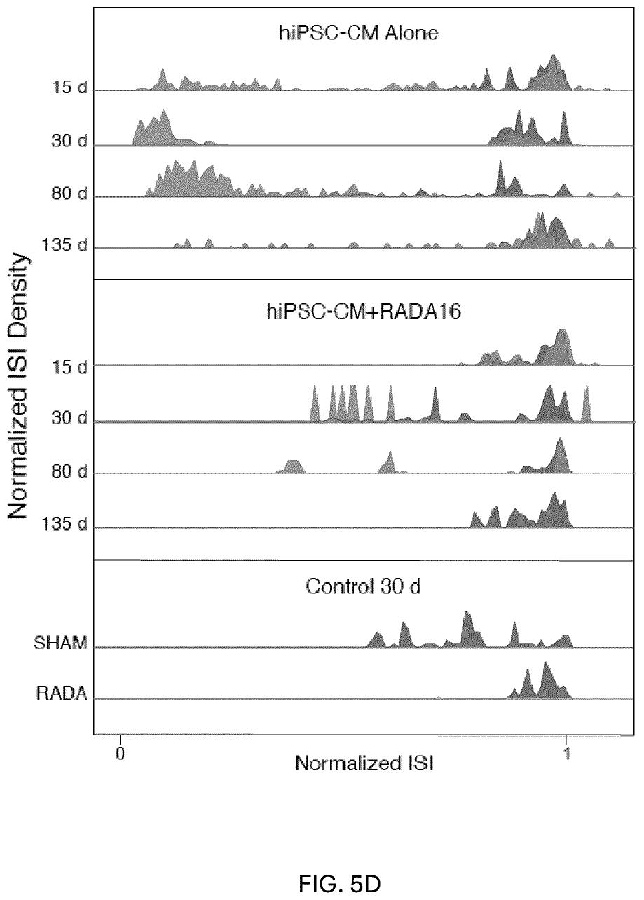

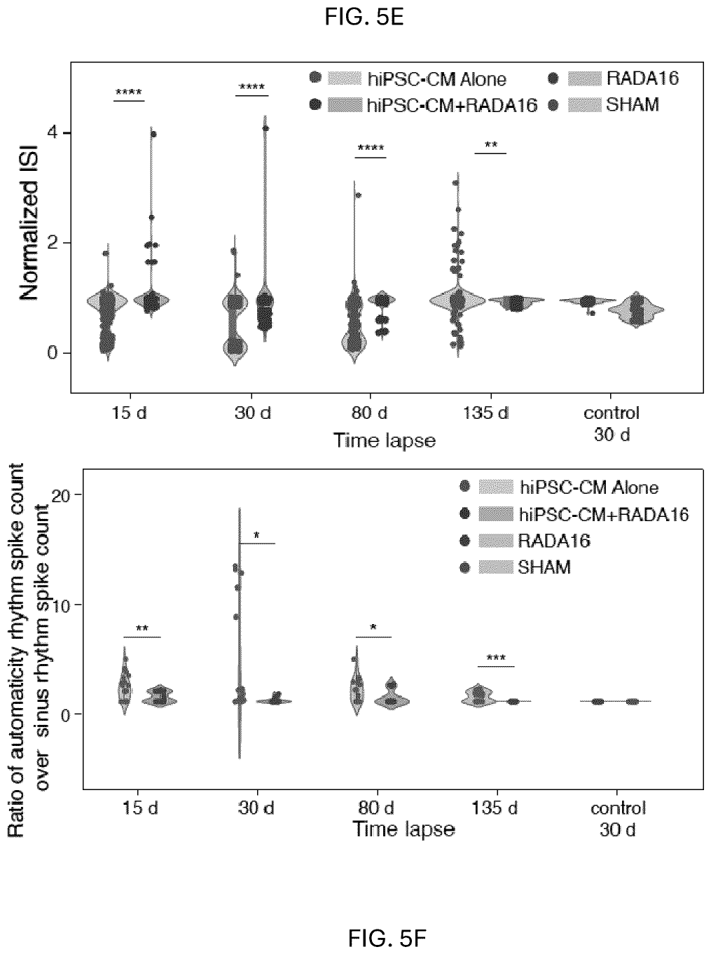

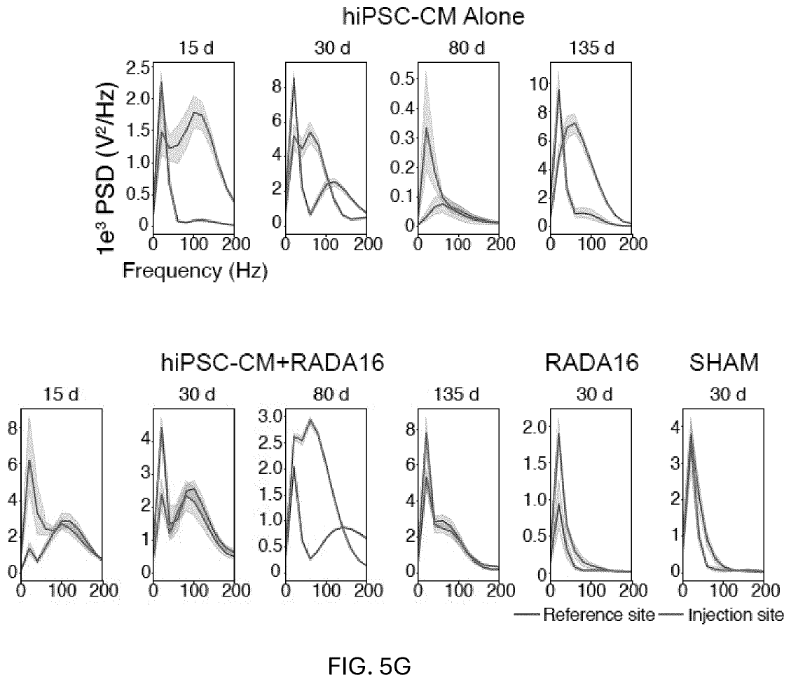

These findings suggest that integrating RADA16 with hiPSC-CMs enhances both structural and functional integration of hiPSC-CMs into host tissues. To systematically assess this, the inventors conducted a longitudinal study of the electrophysiological adaptations induced by RADA16 in hiPSC-CMs over time. Three groups of samples were studied (FIG. 5A): control groups of animals injected with either RADA16 or SHAM, a group injected with hiPSC-CM alone (hiPSC-CM Alone), and a group injected with hiPSC-CMs combined with RADA16 (hiPSC-CM+RAD16). Control groups were recorded at 30-day post-injection, while the latter two groups were recorded at 15-, 30-, 80- and 135-day post-injection.

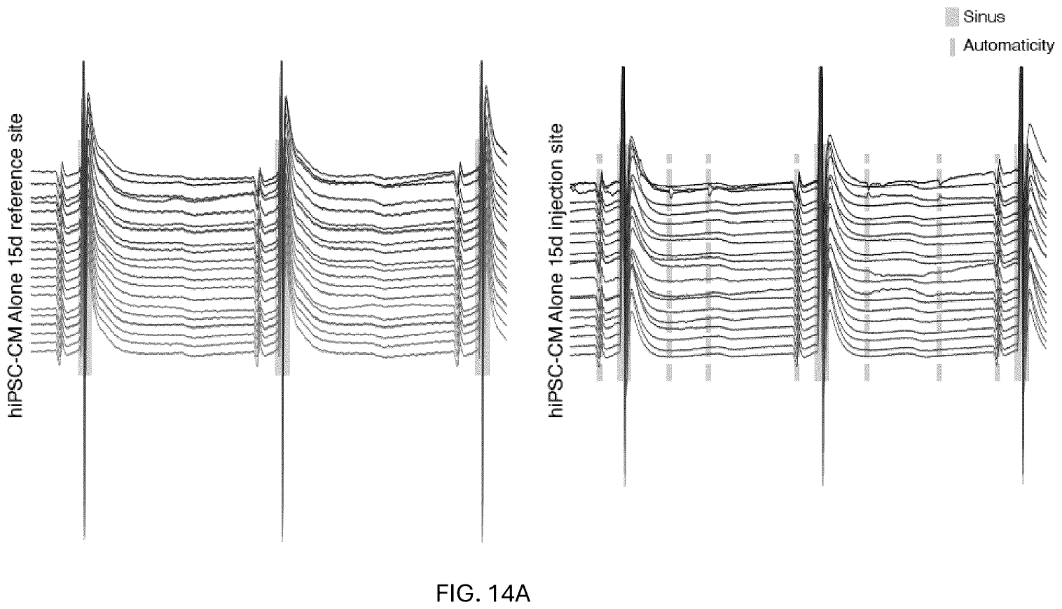

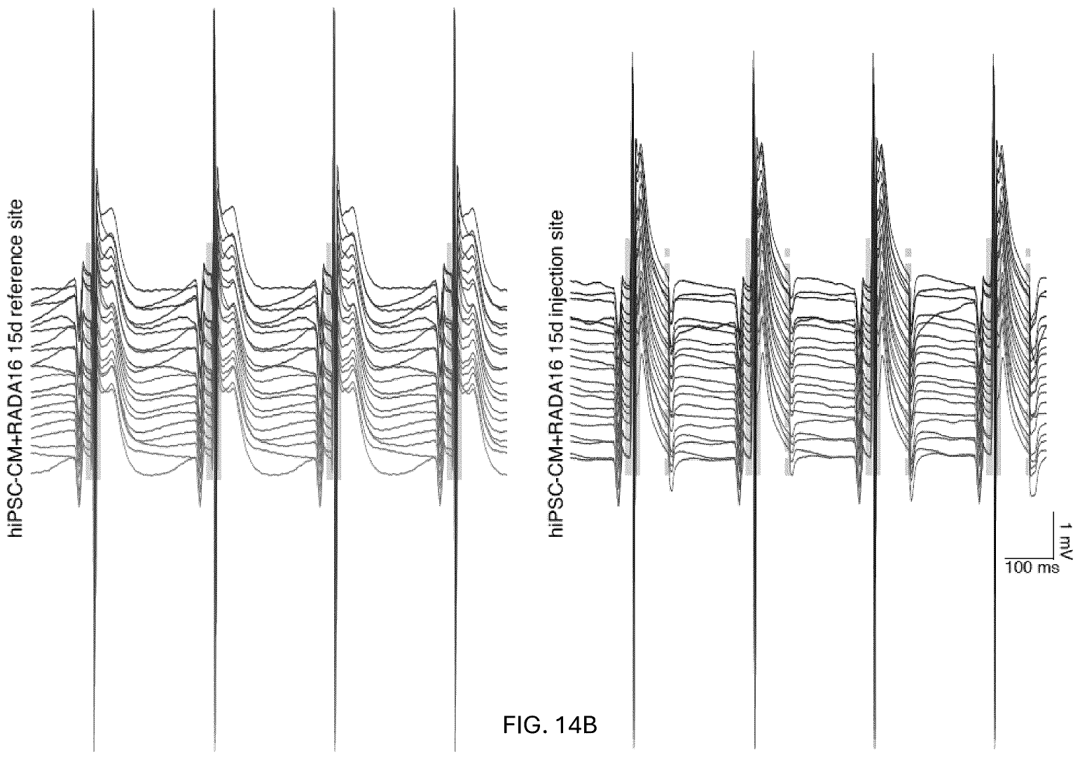

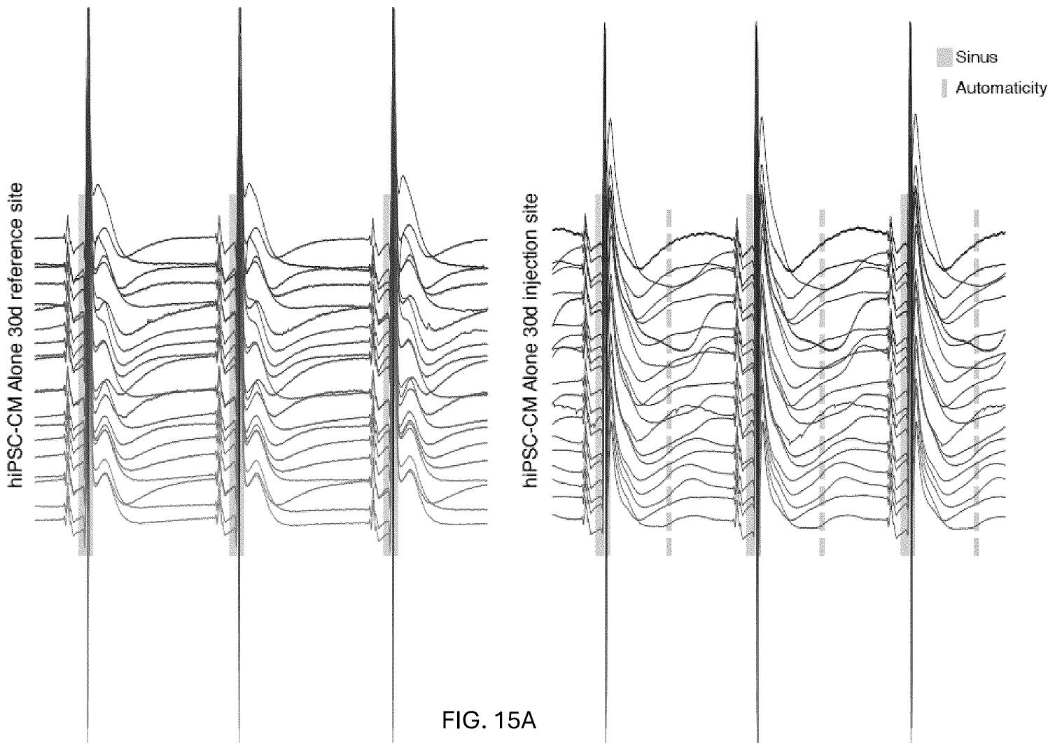

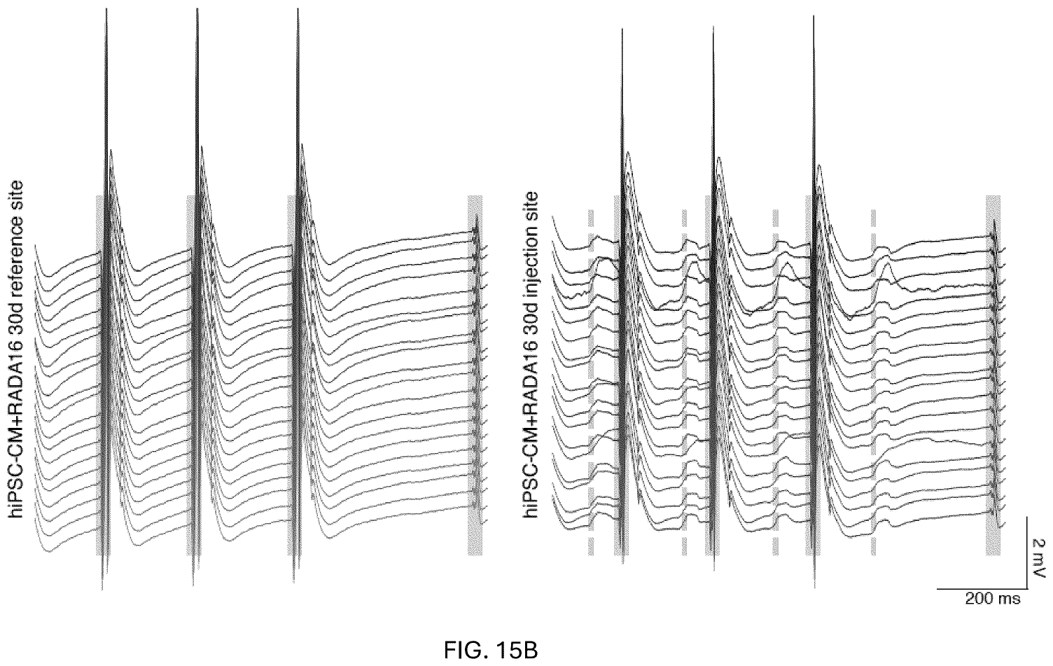

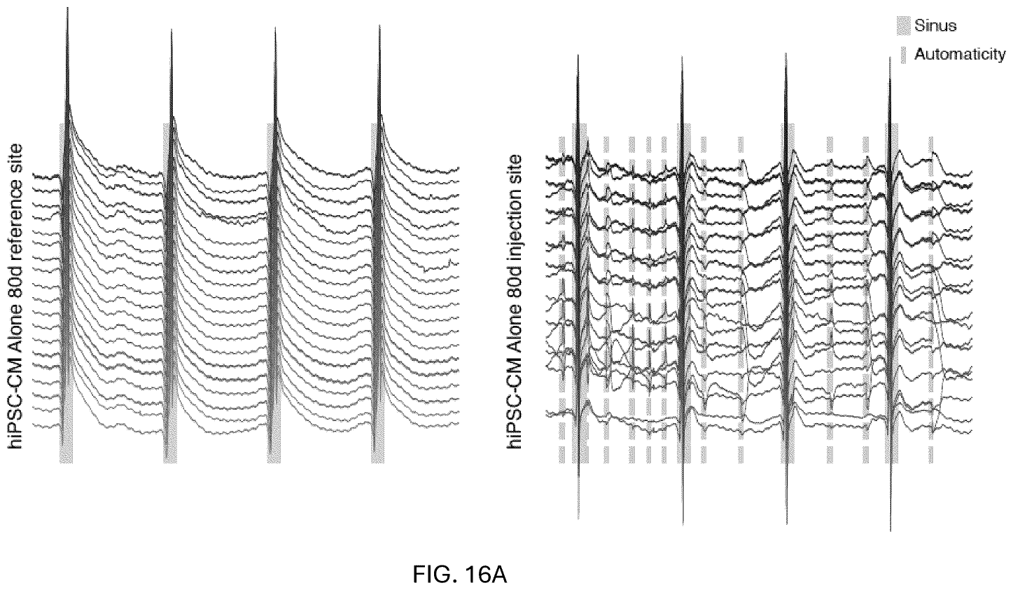

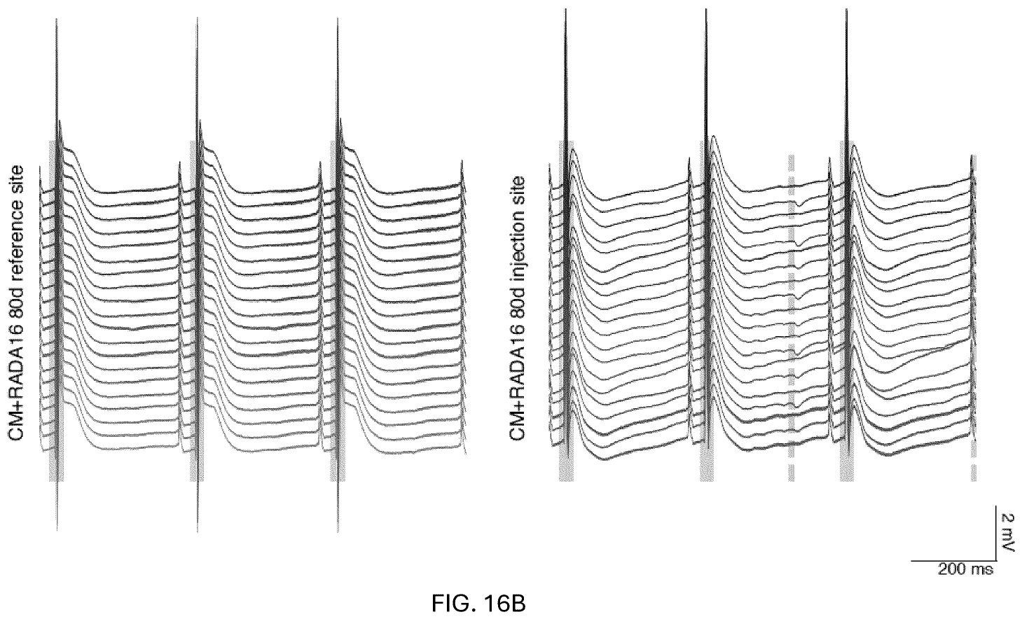

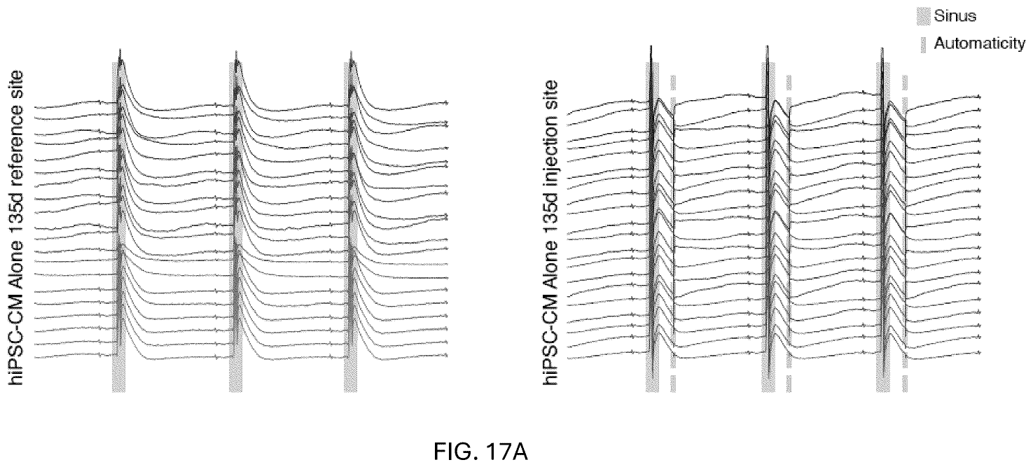

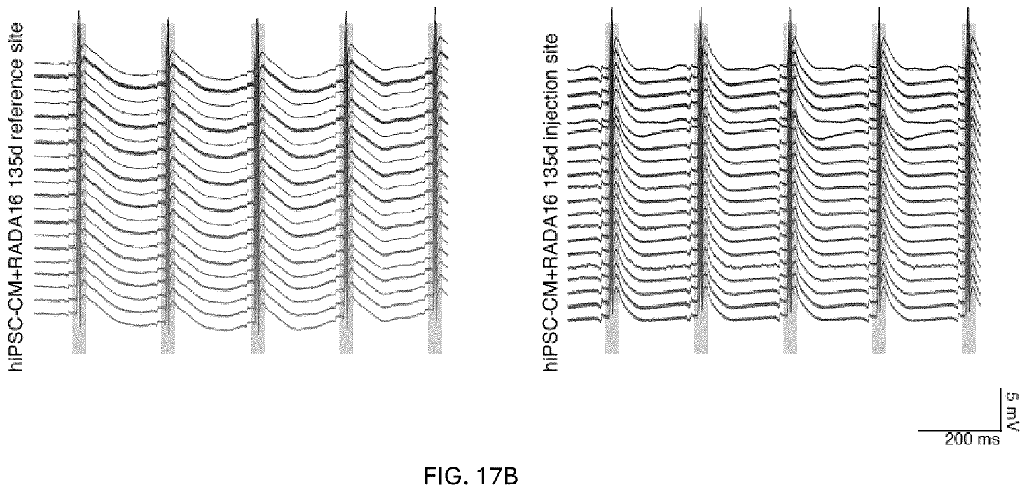

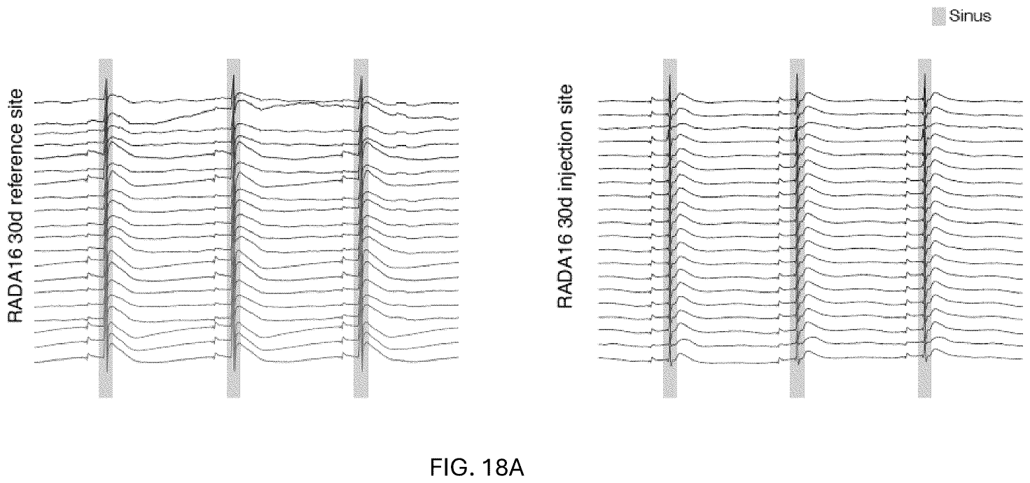

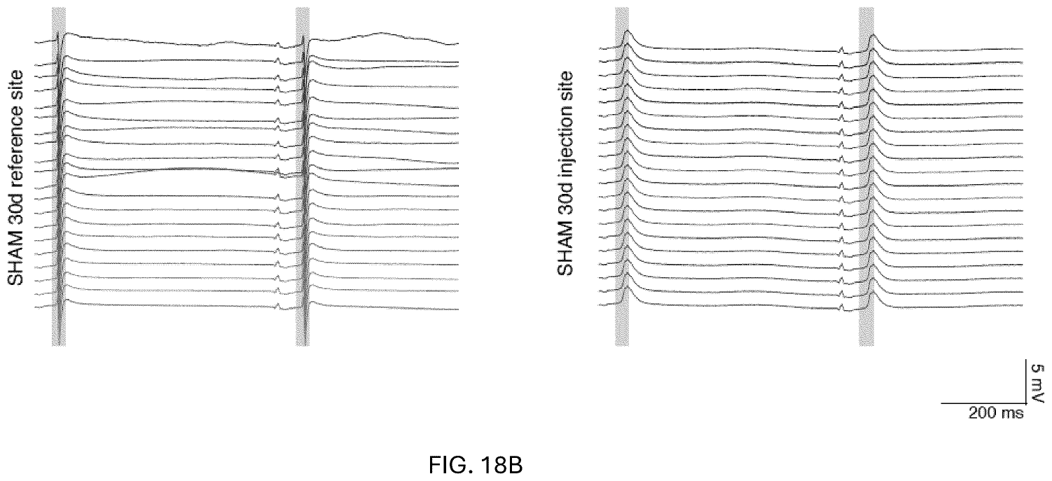

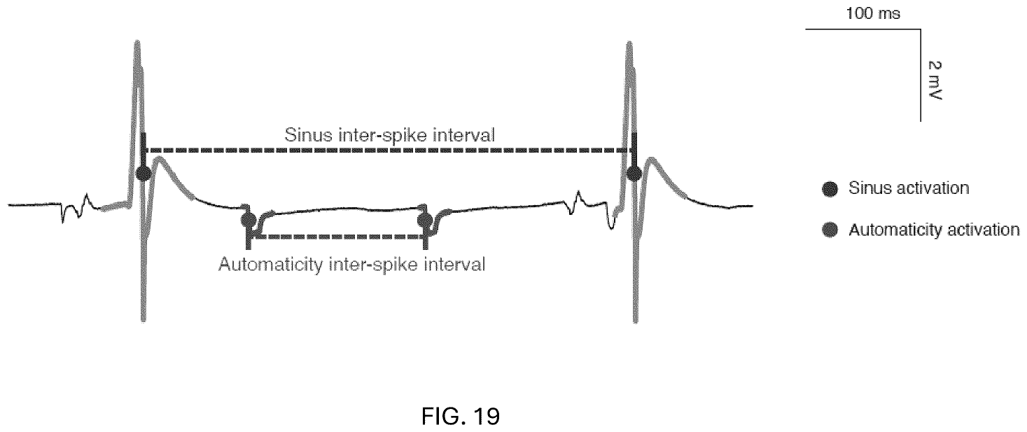

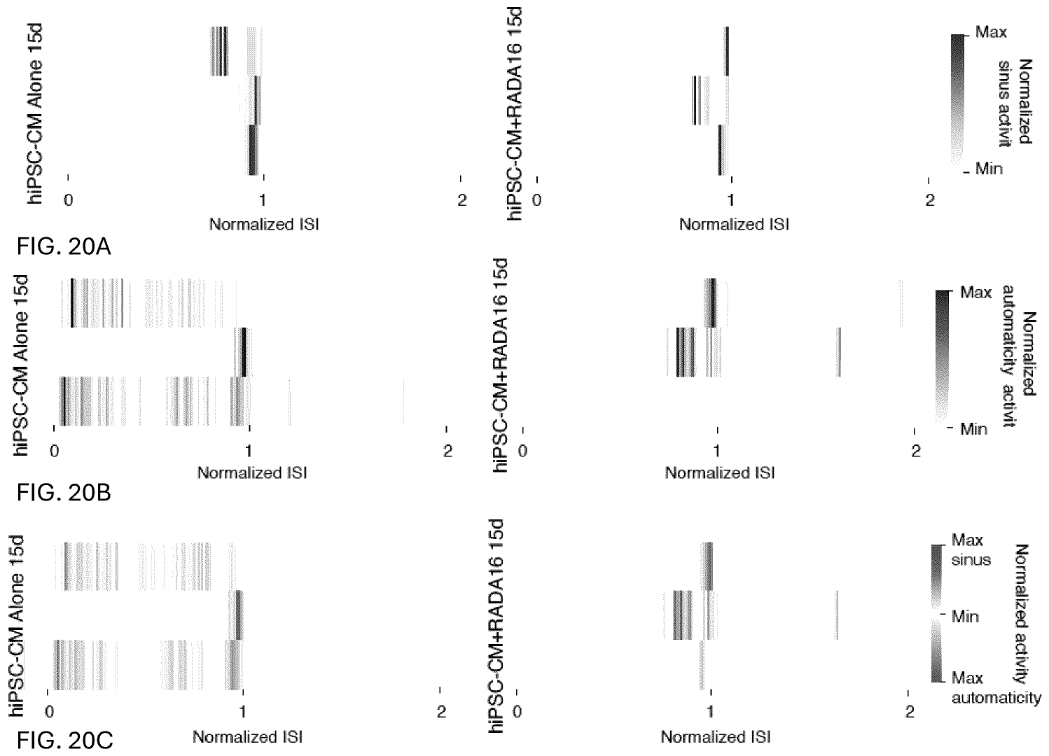

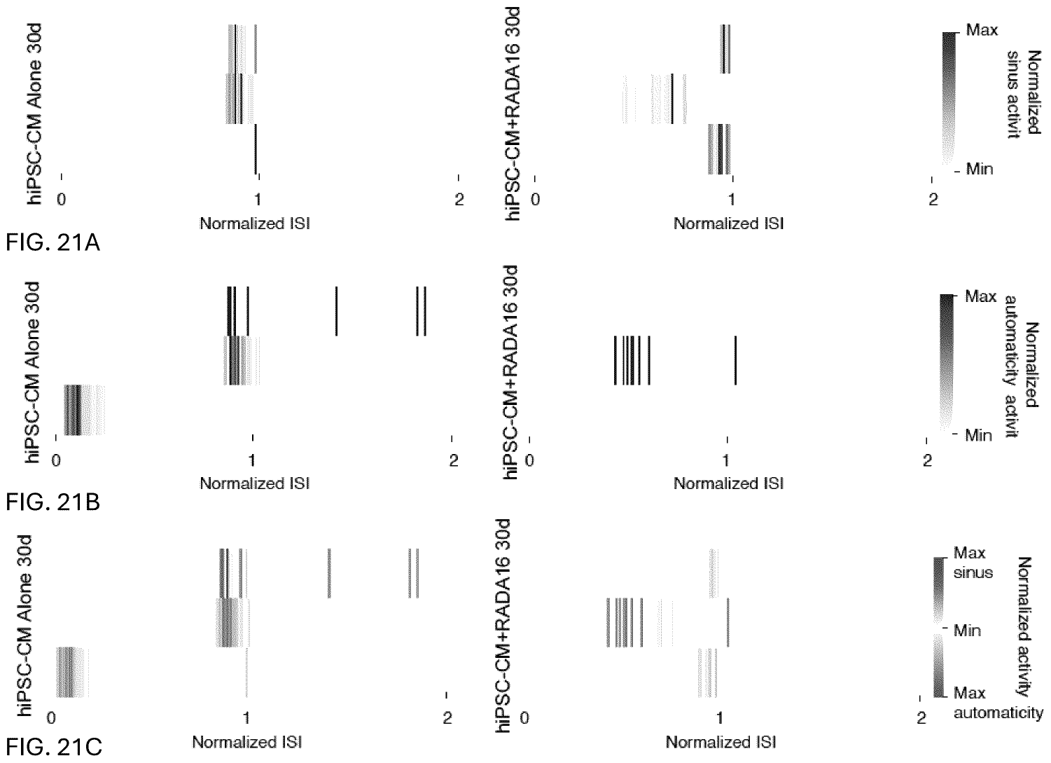

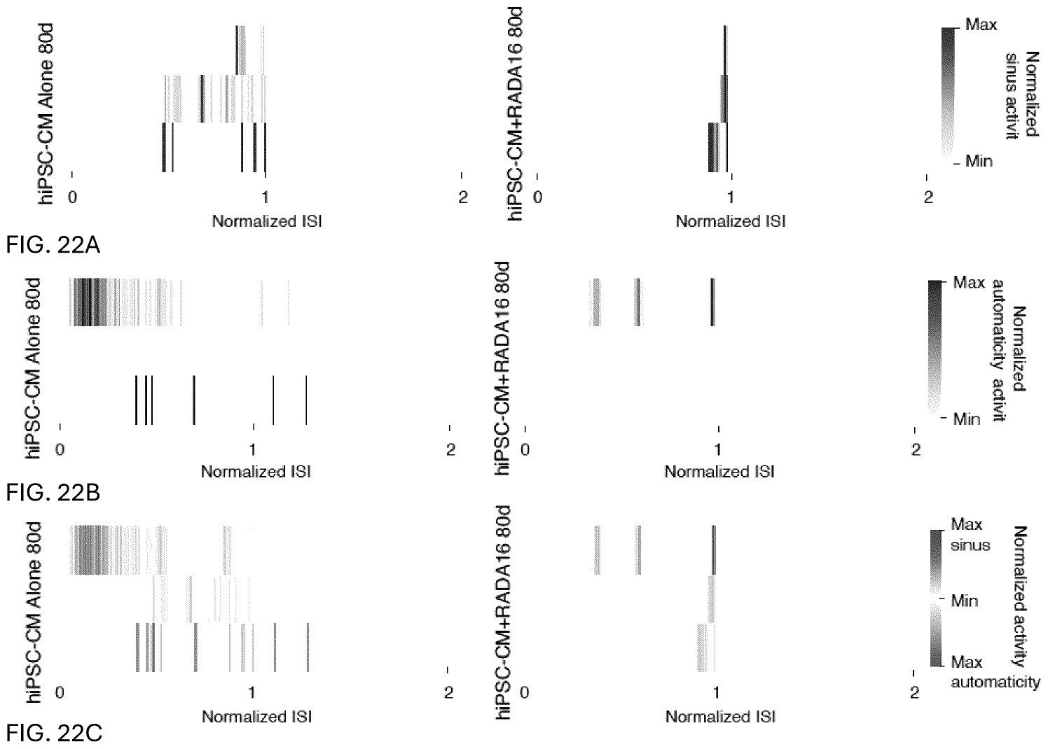

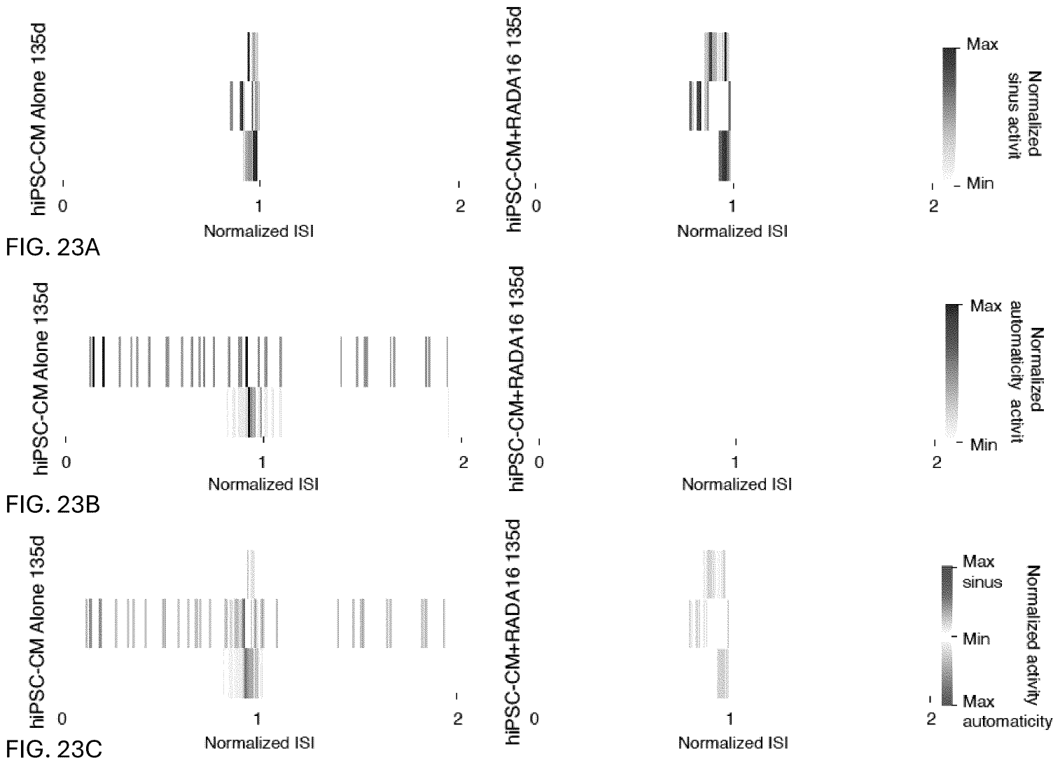

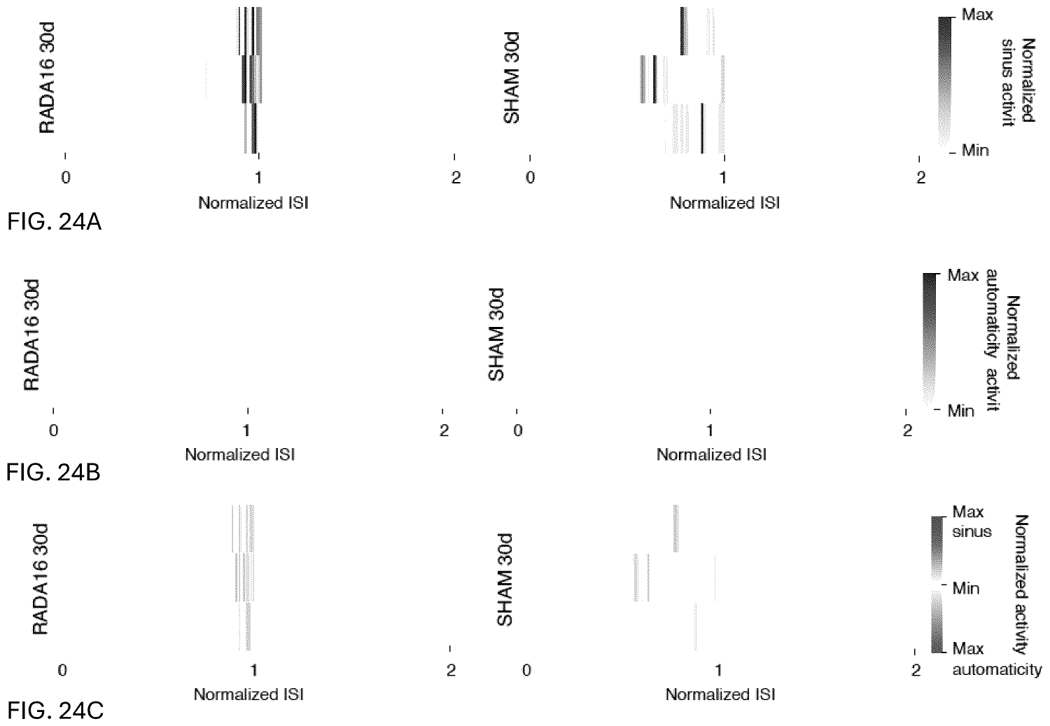

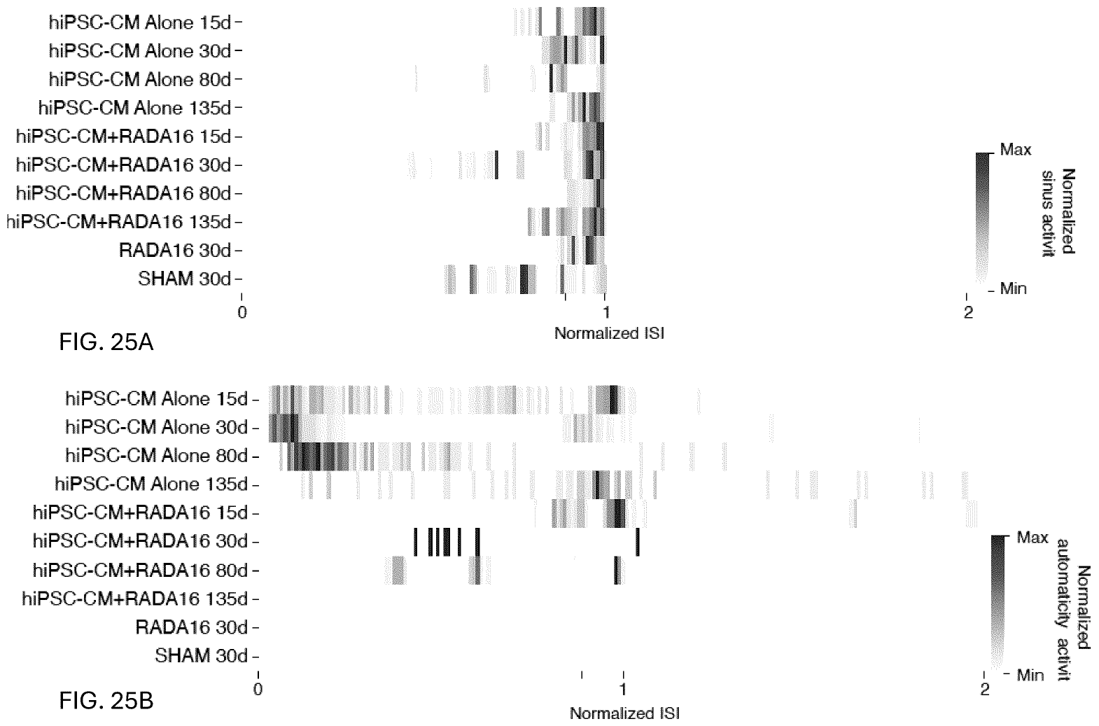

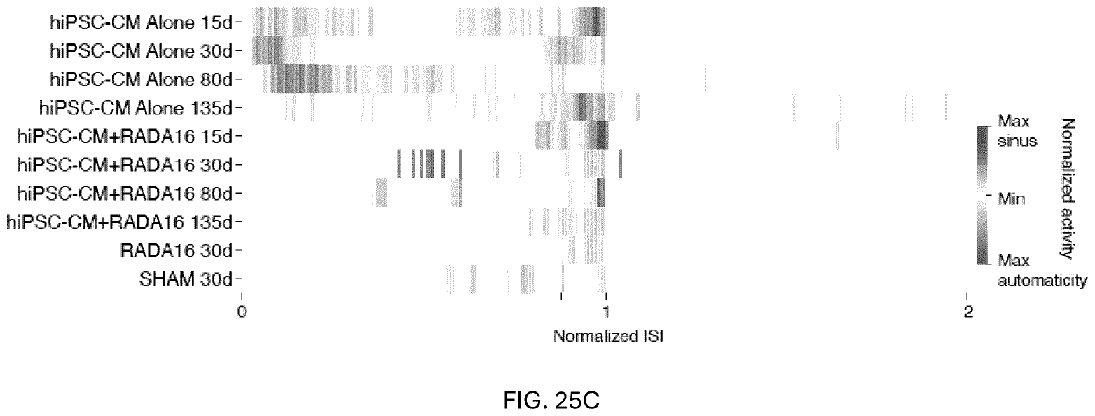

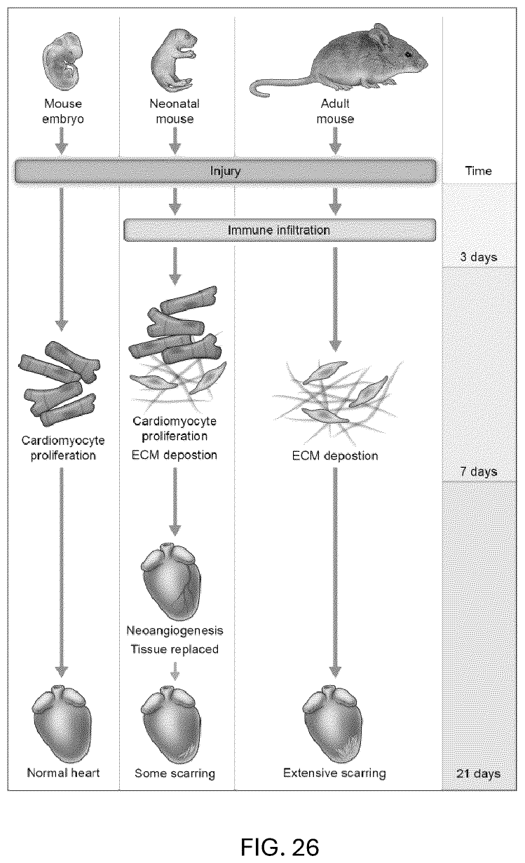

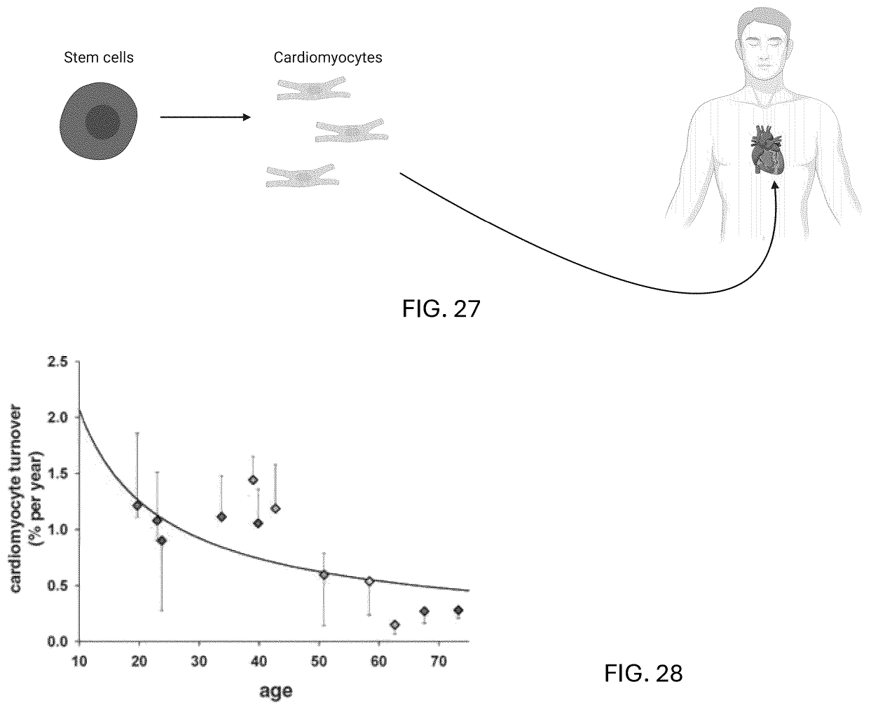

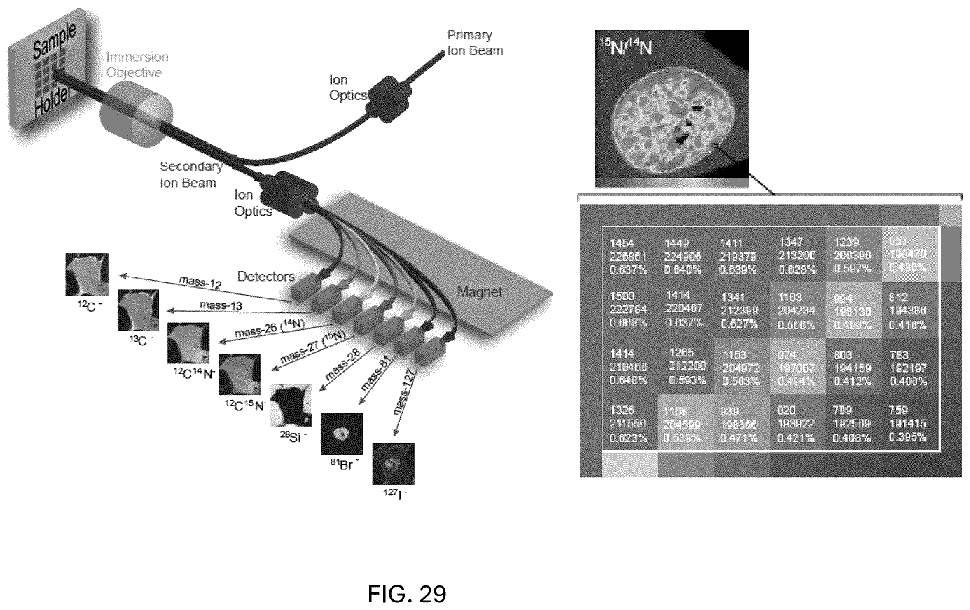

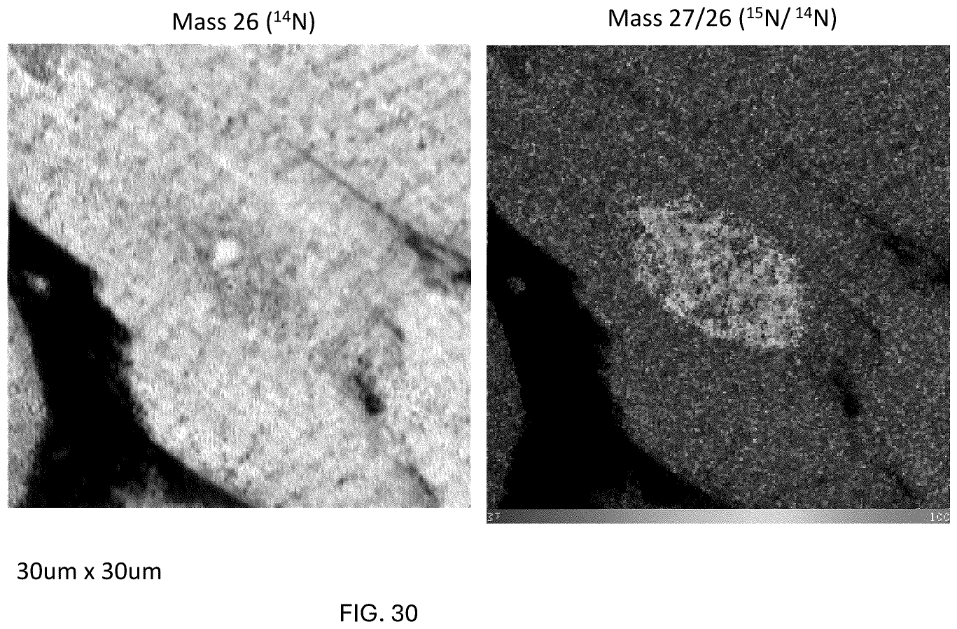

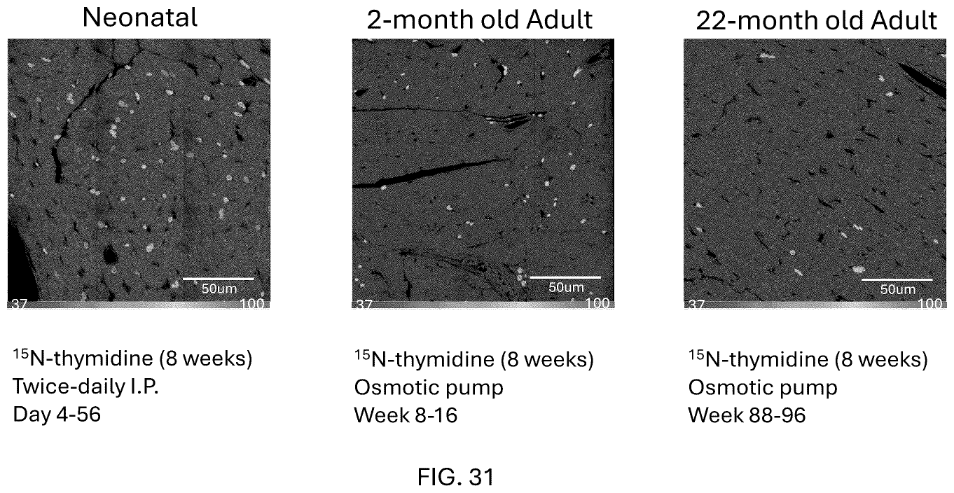

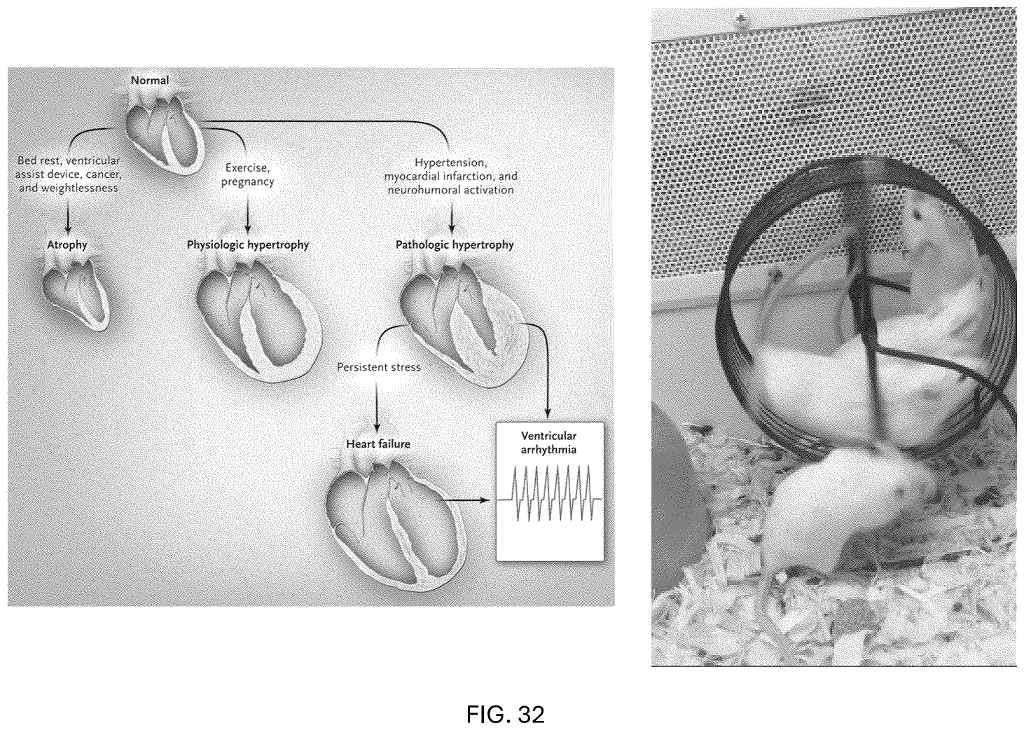

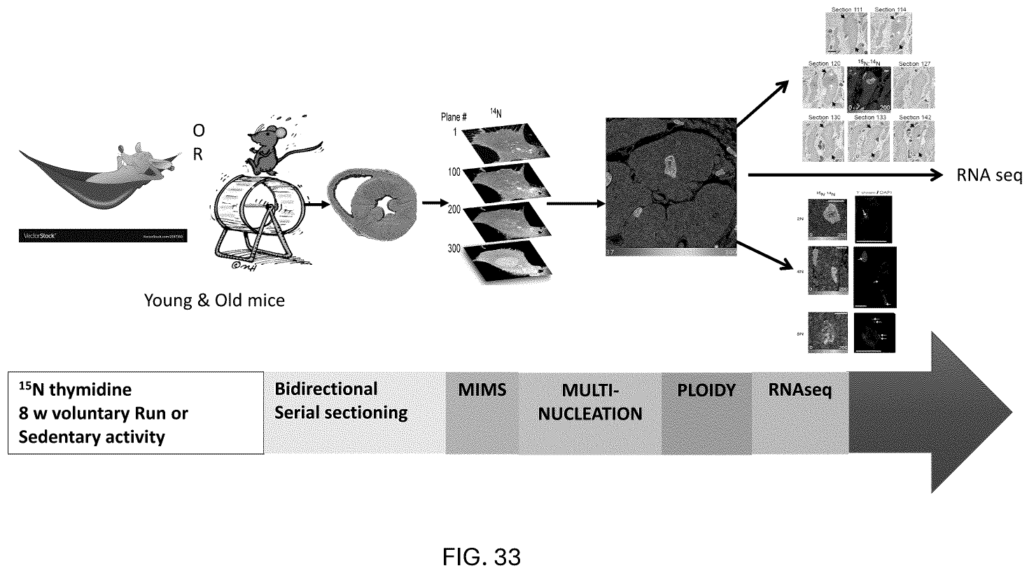

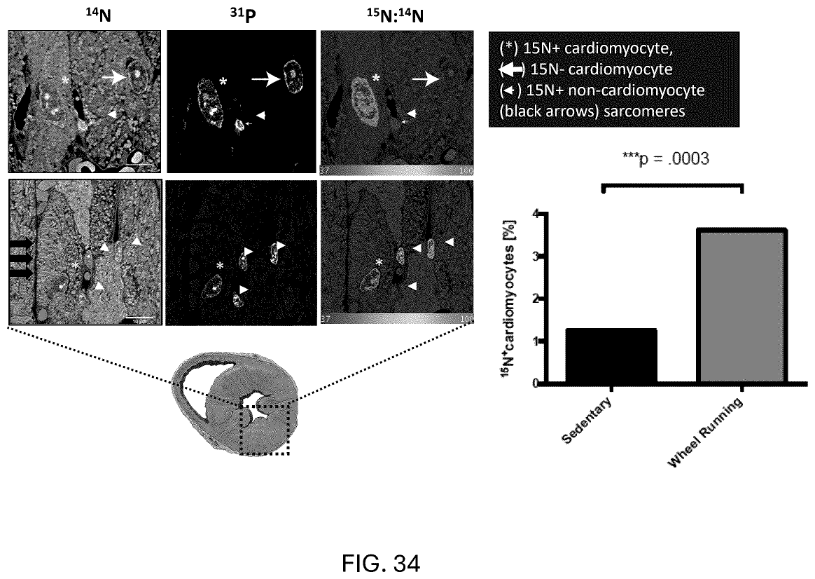

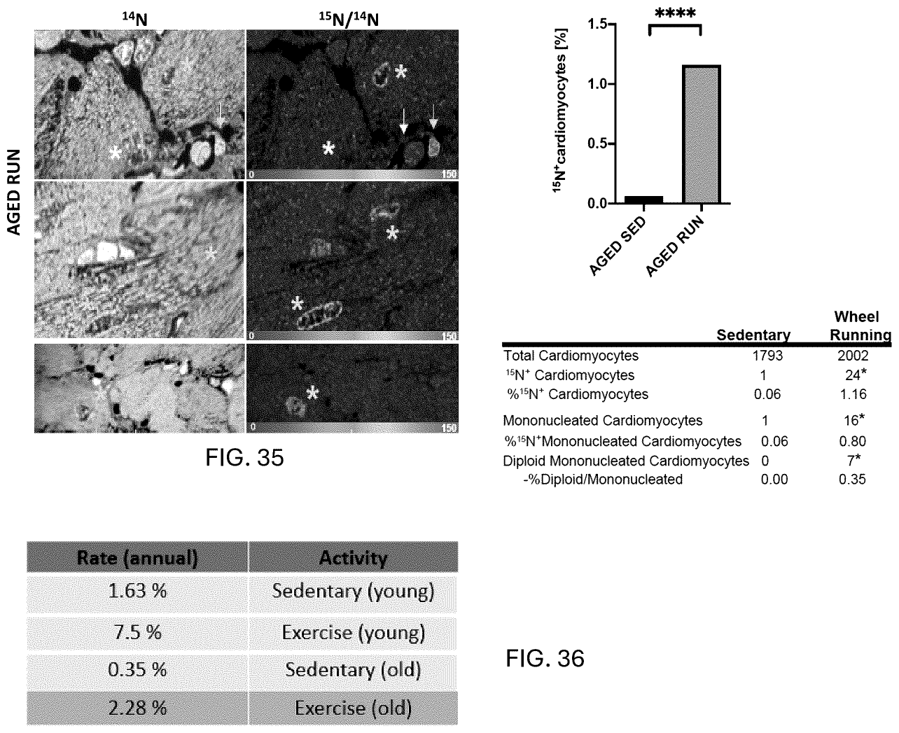

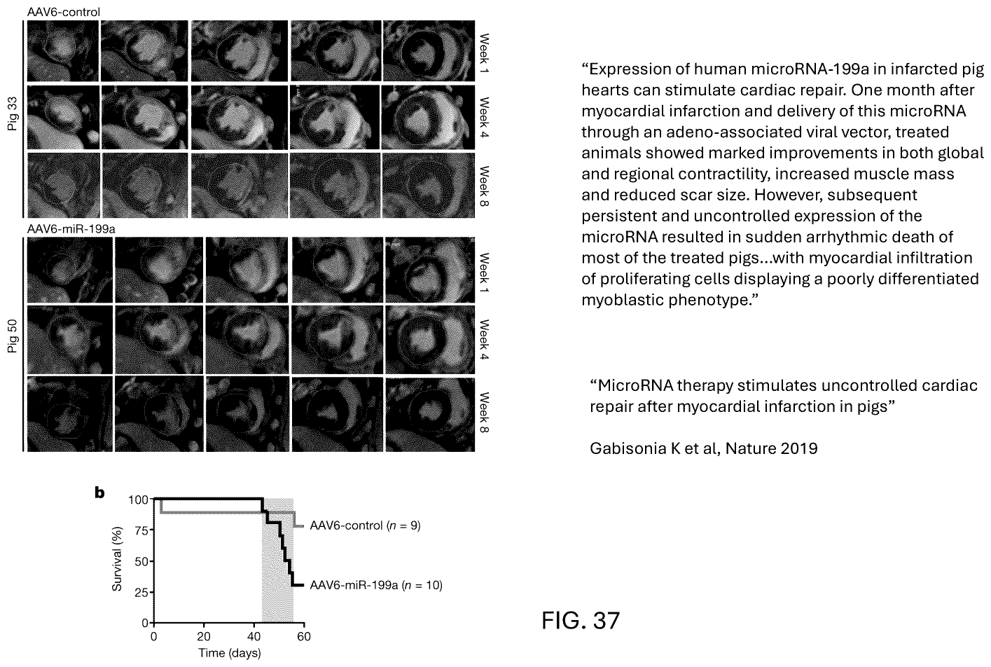

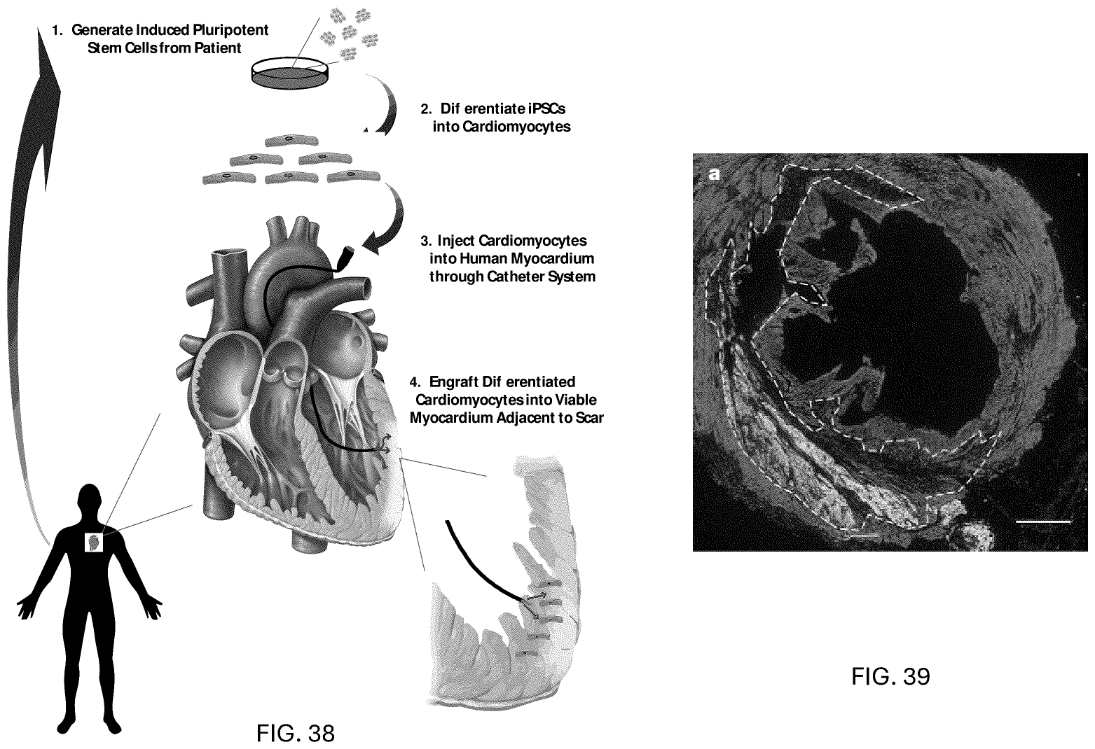

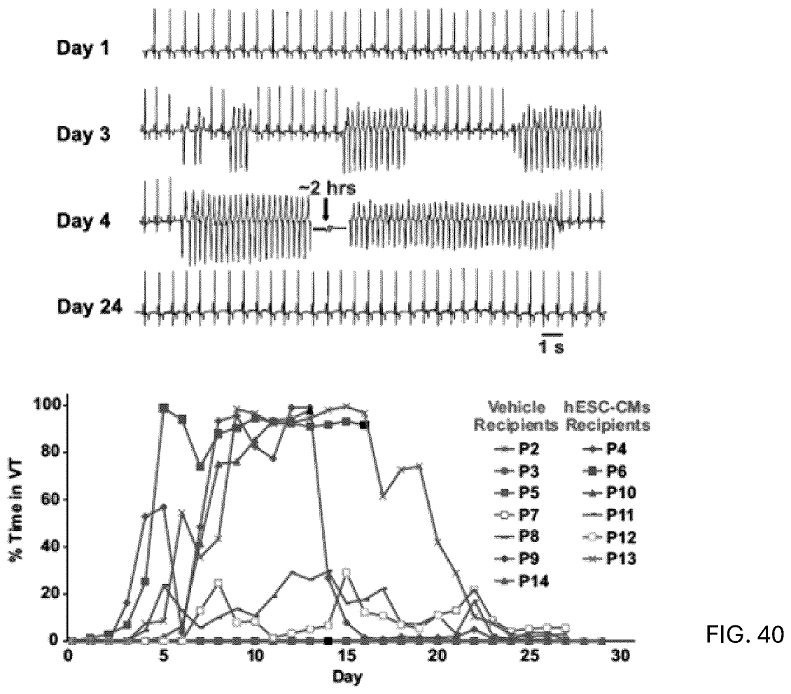

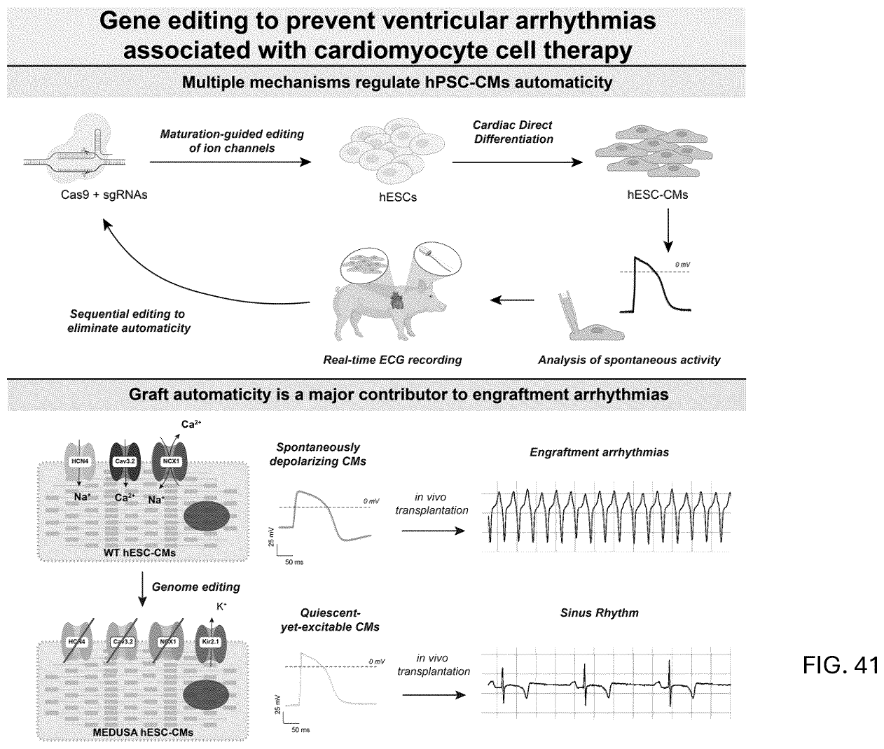

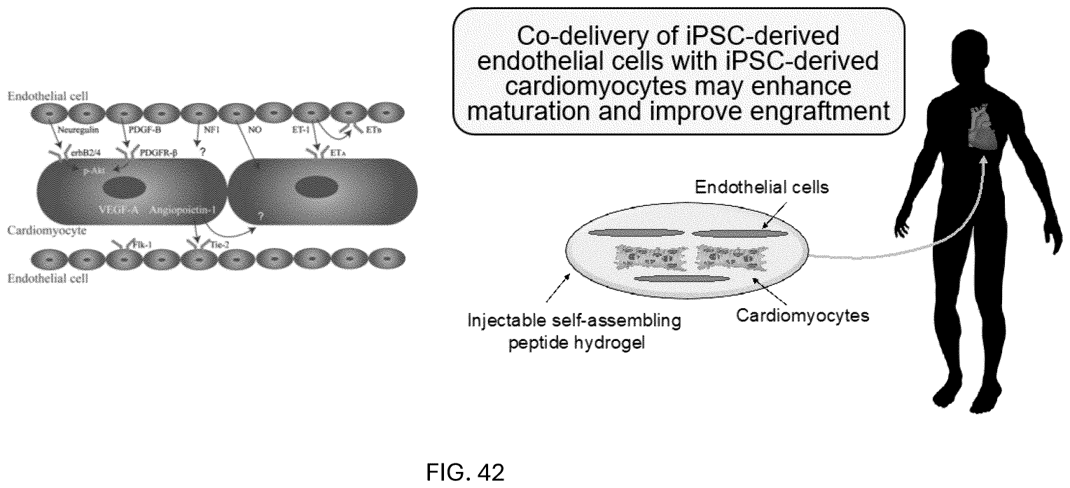

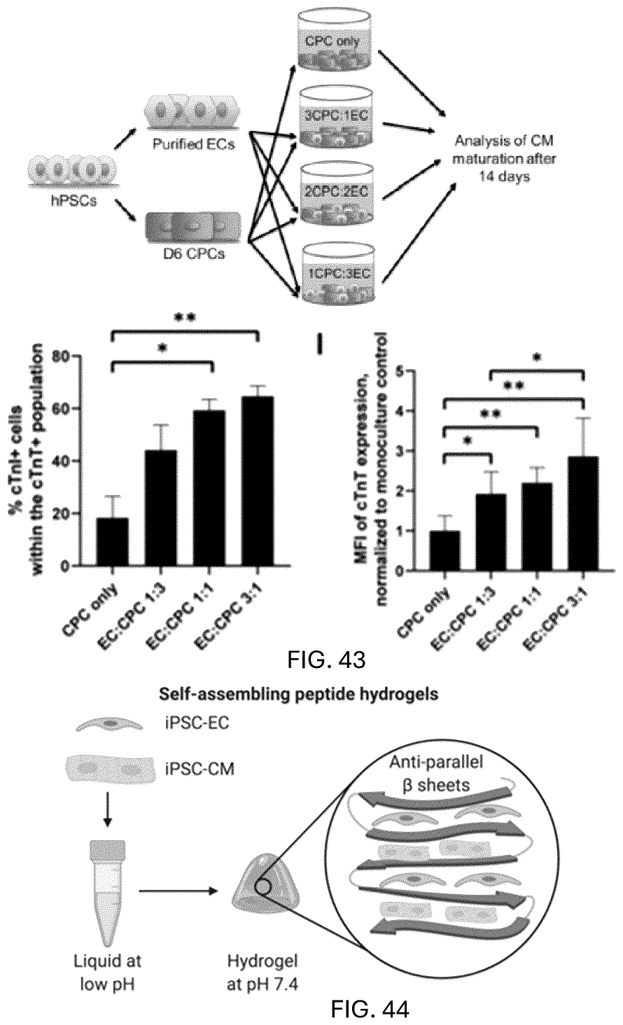

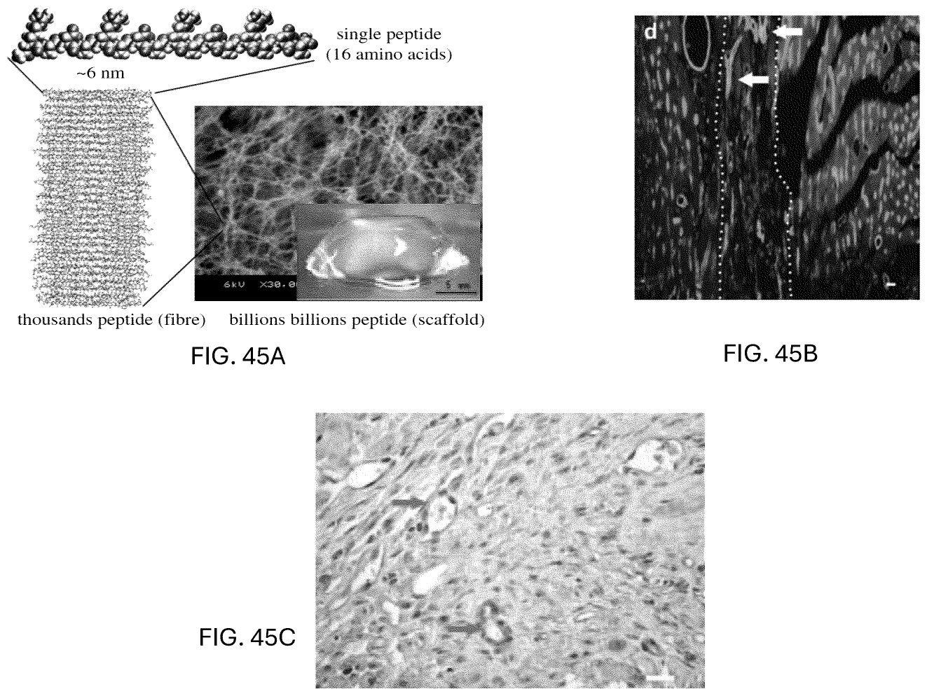

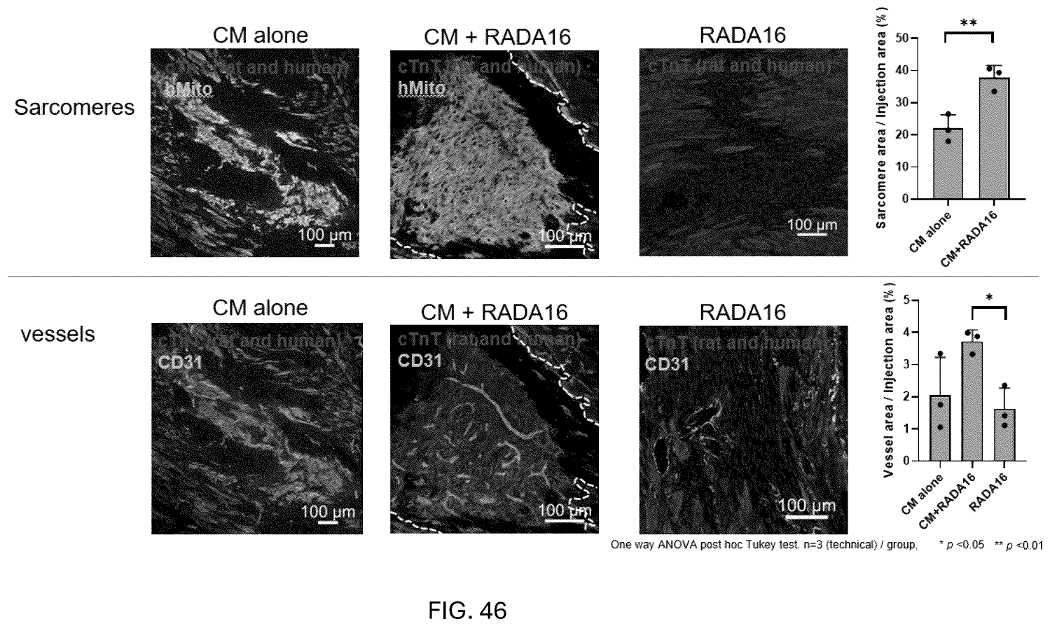

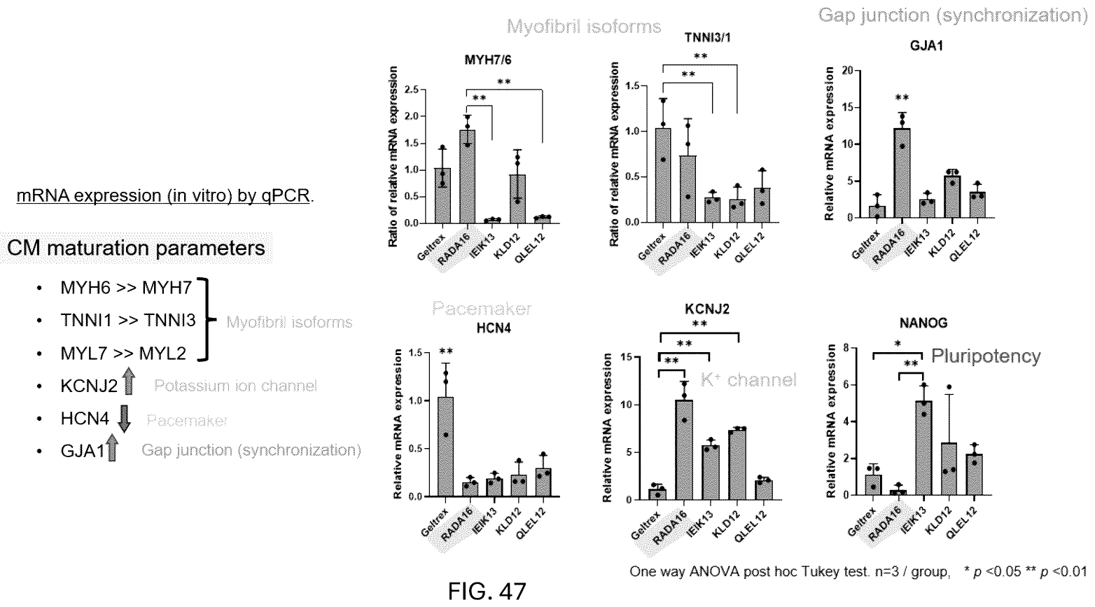

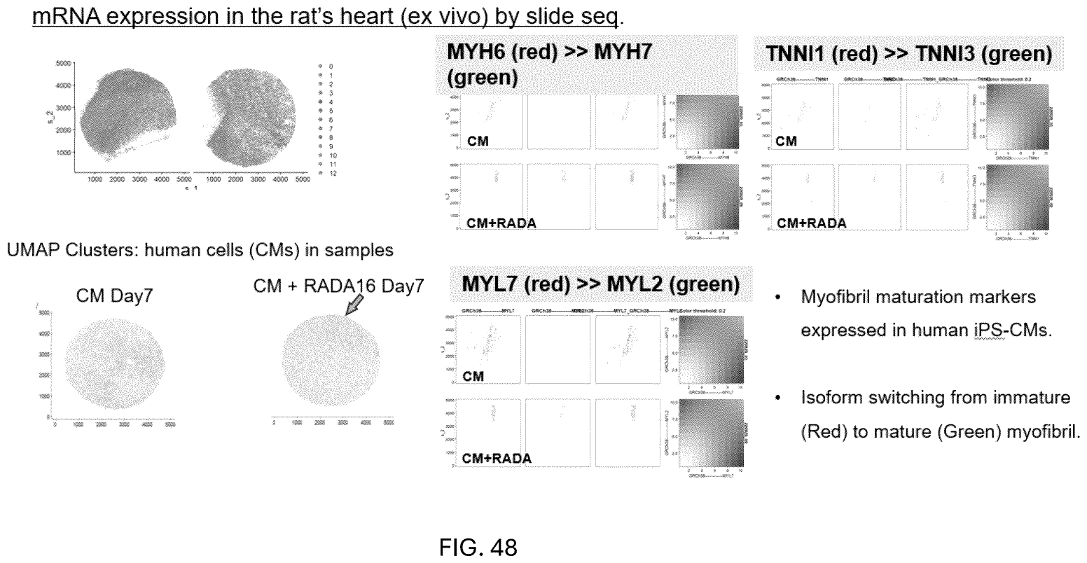

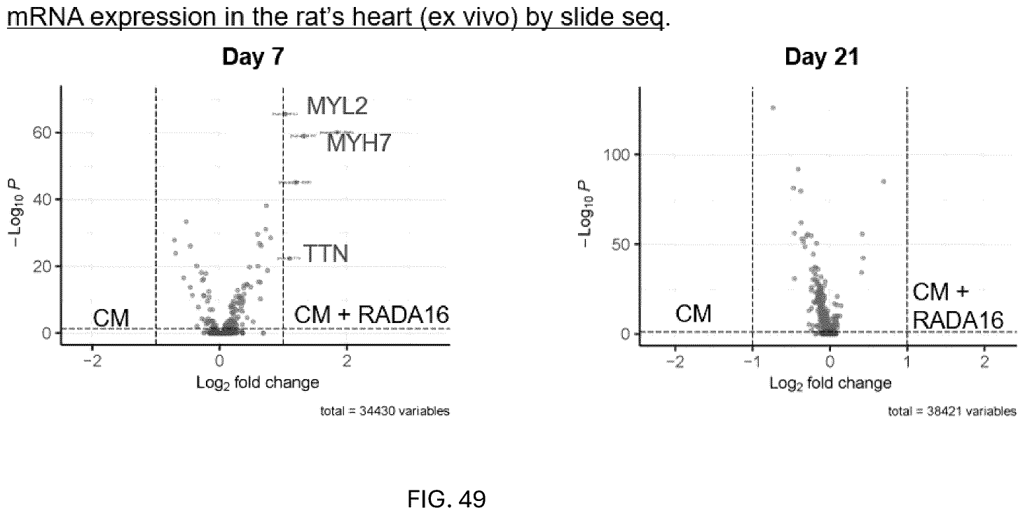

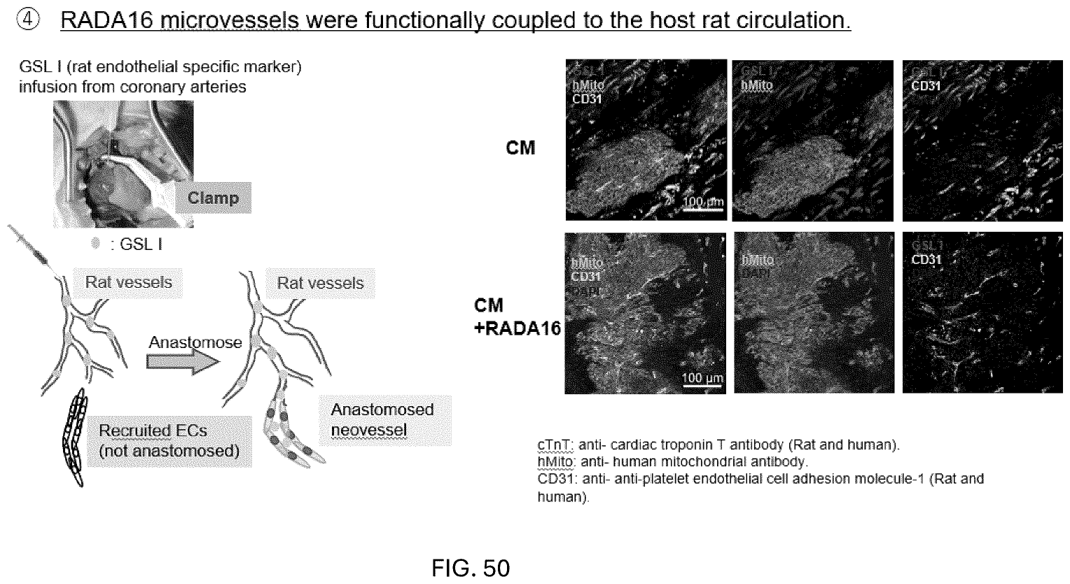

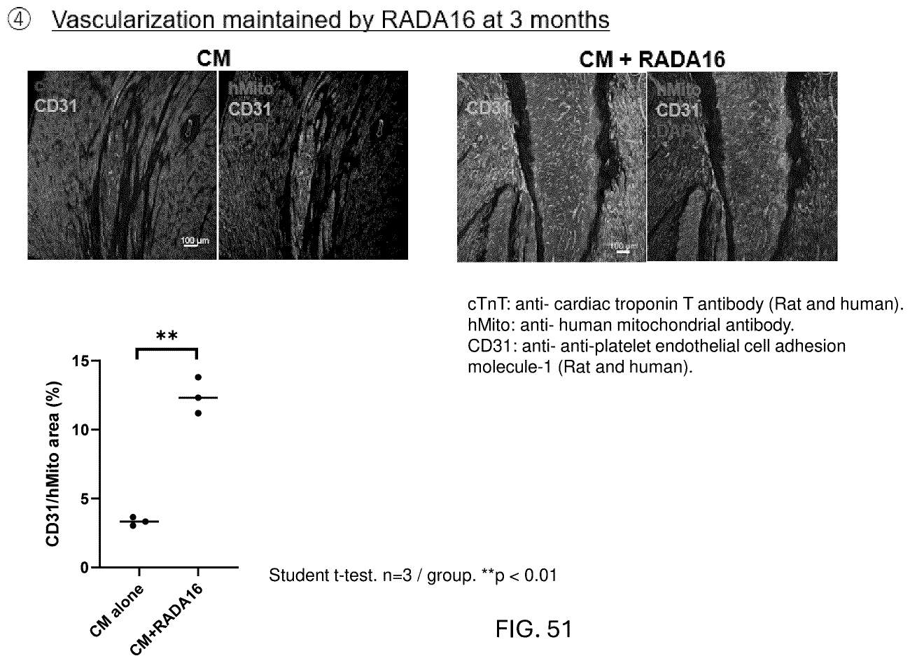

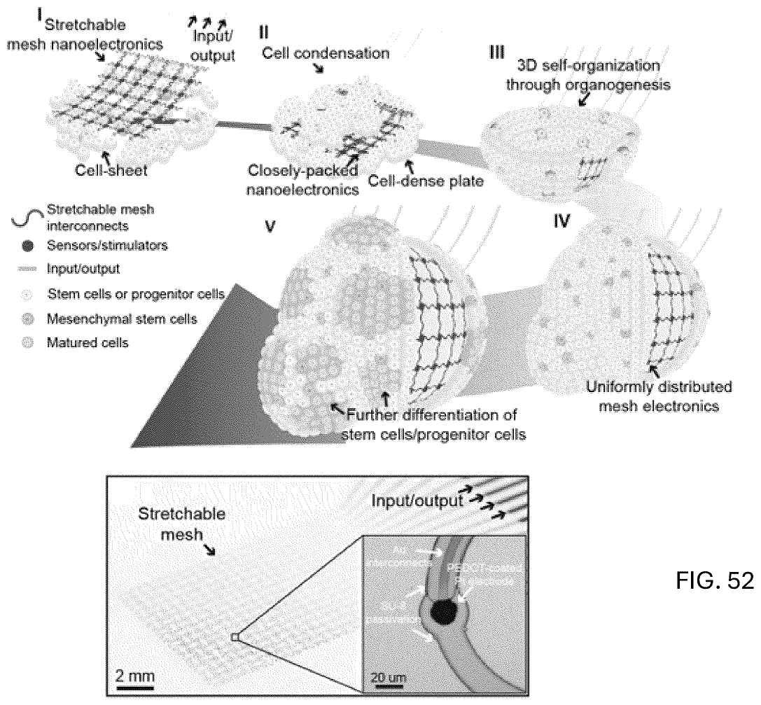

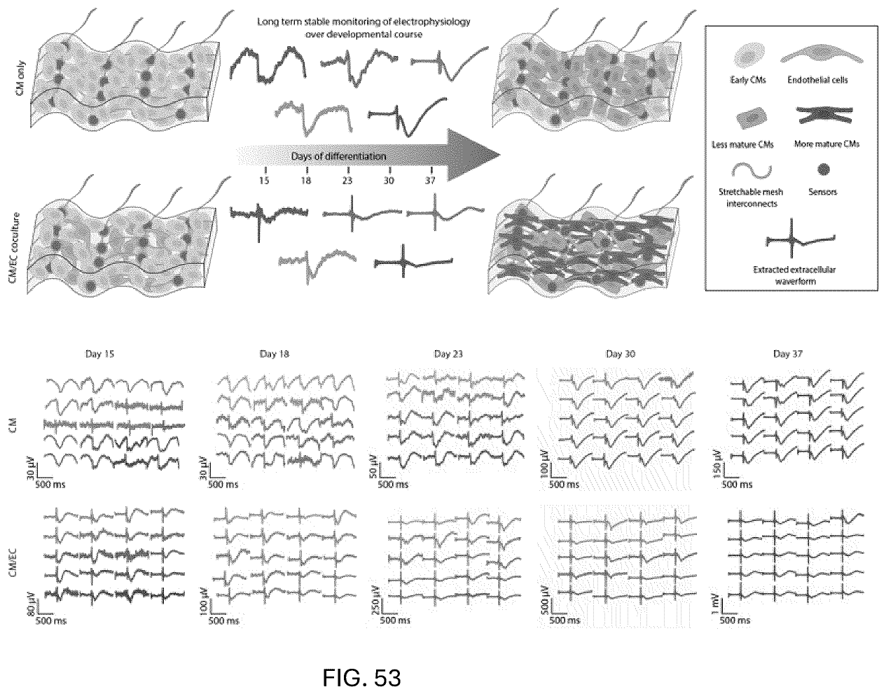

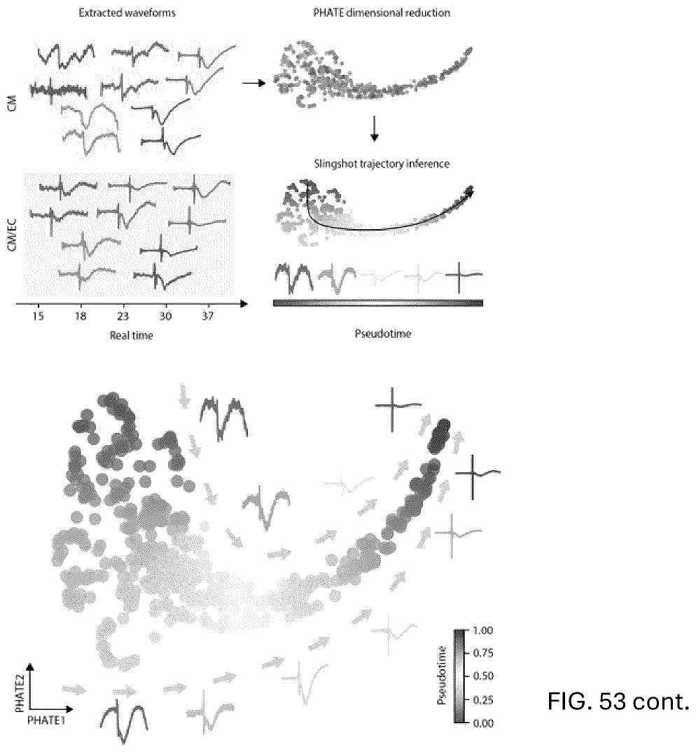

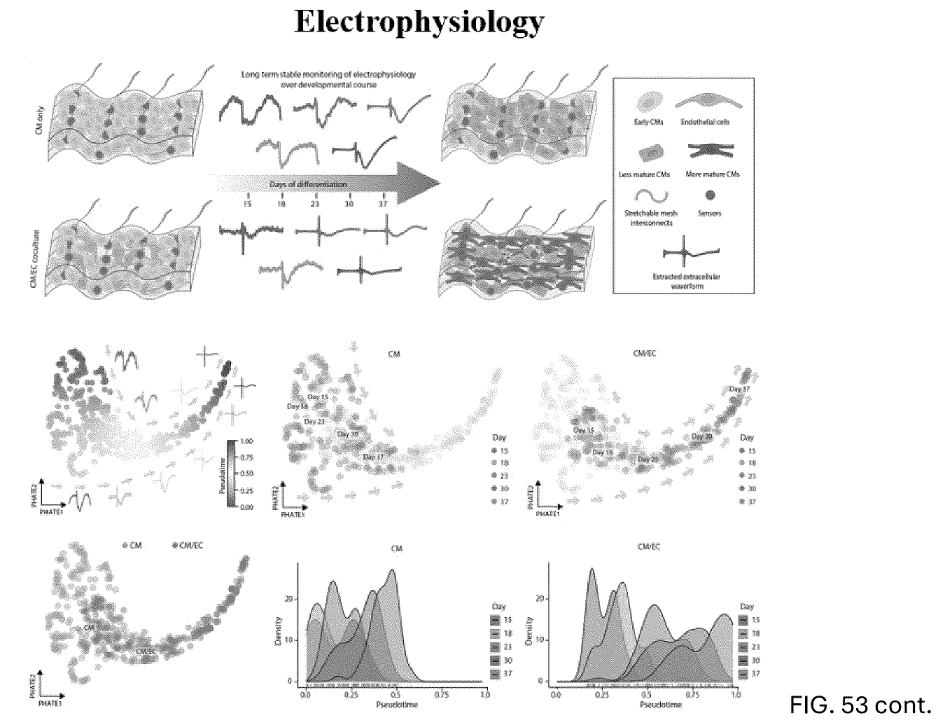

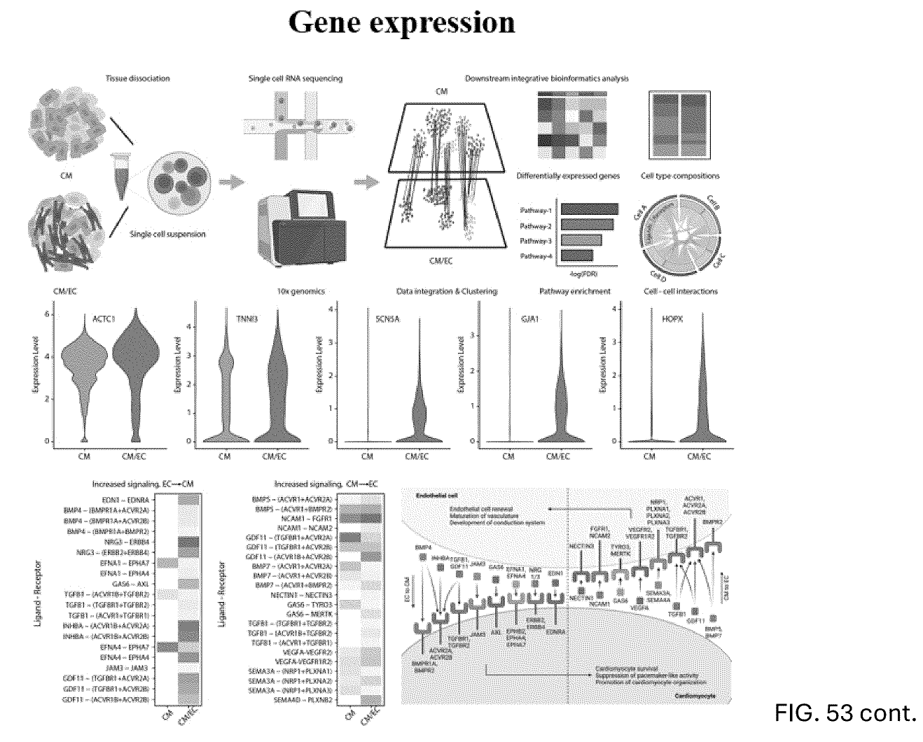

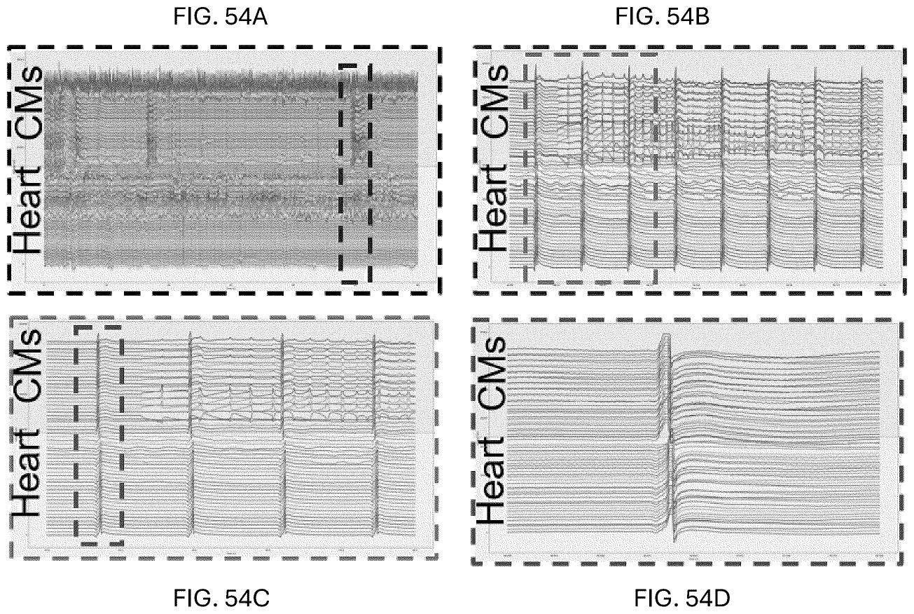

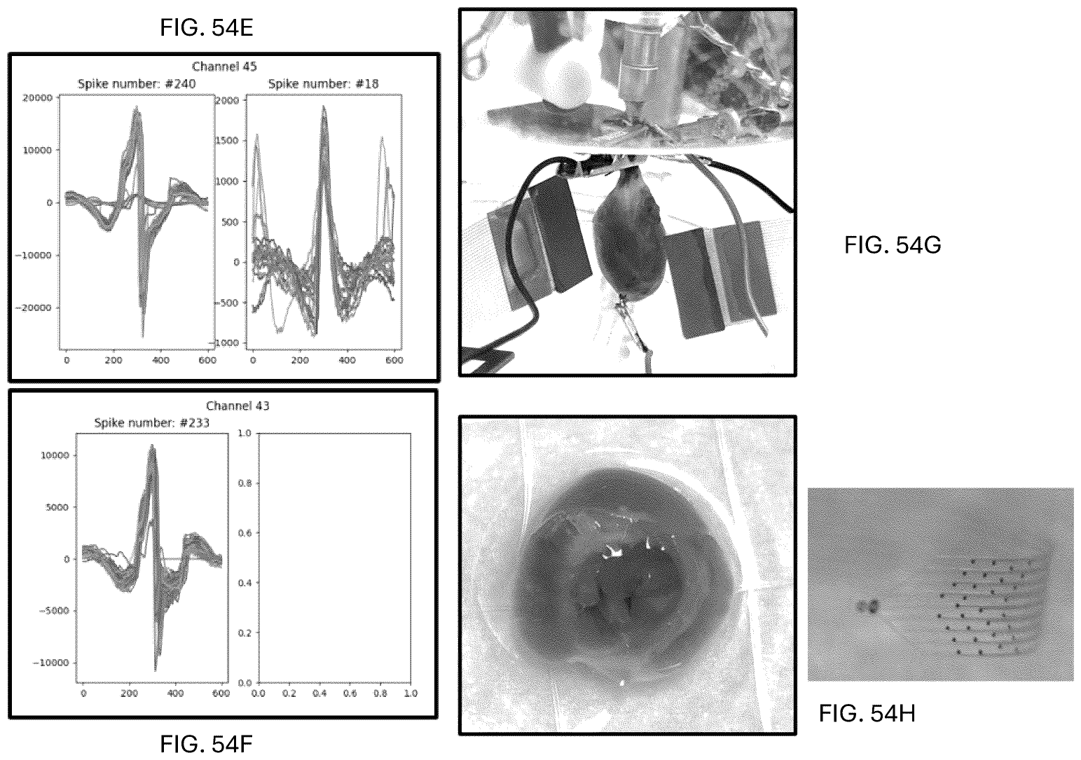

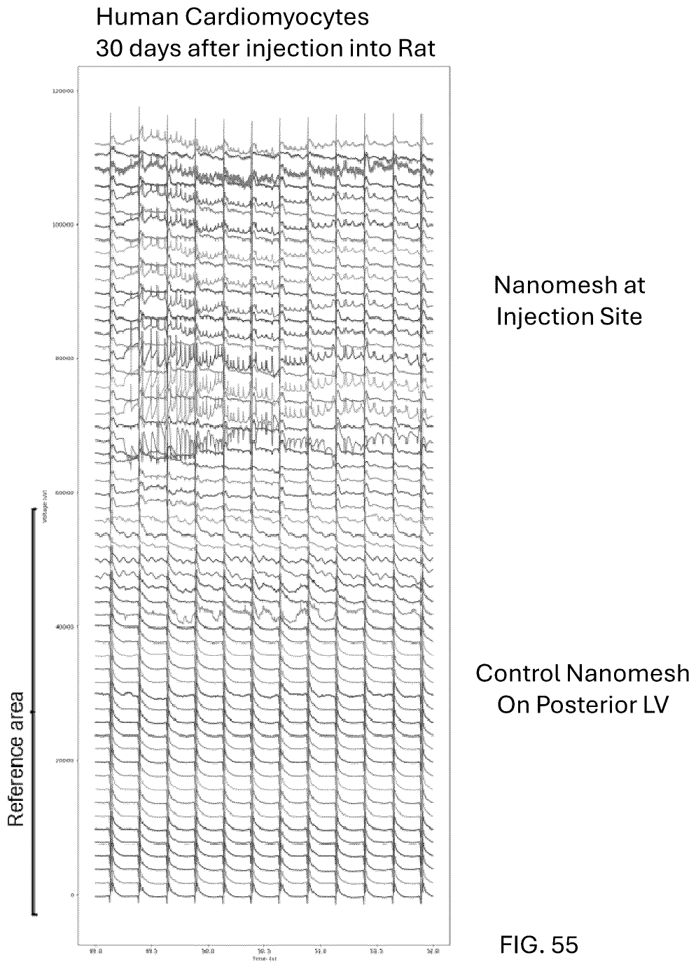

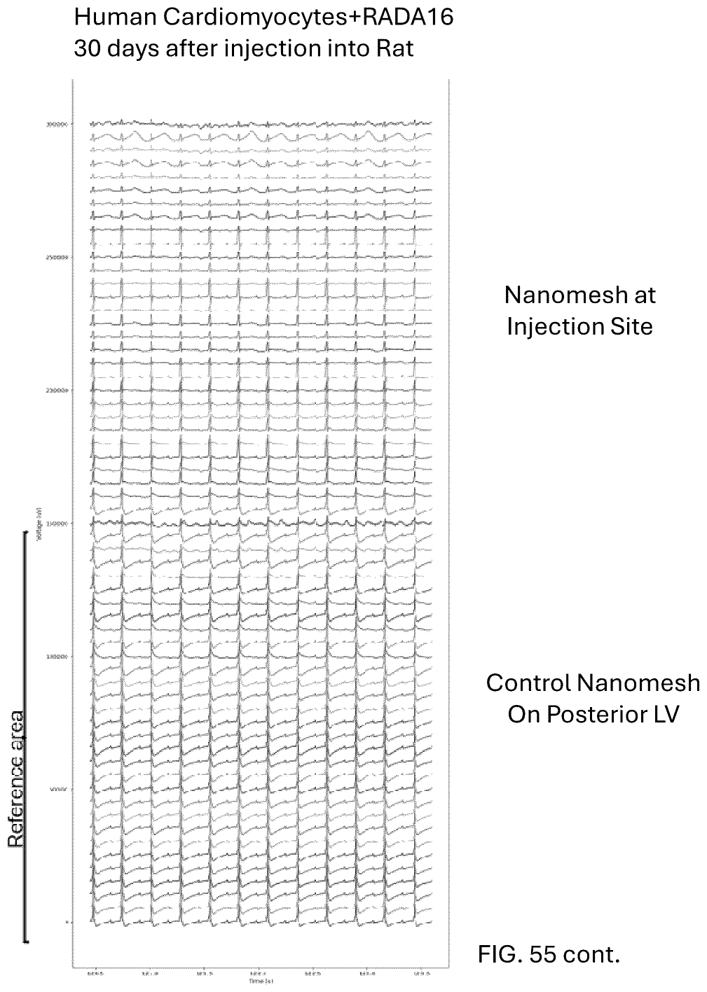

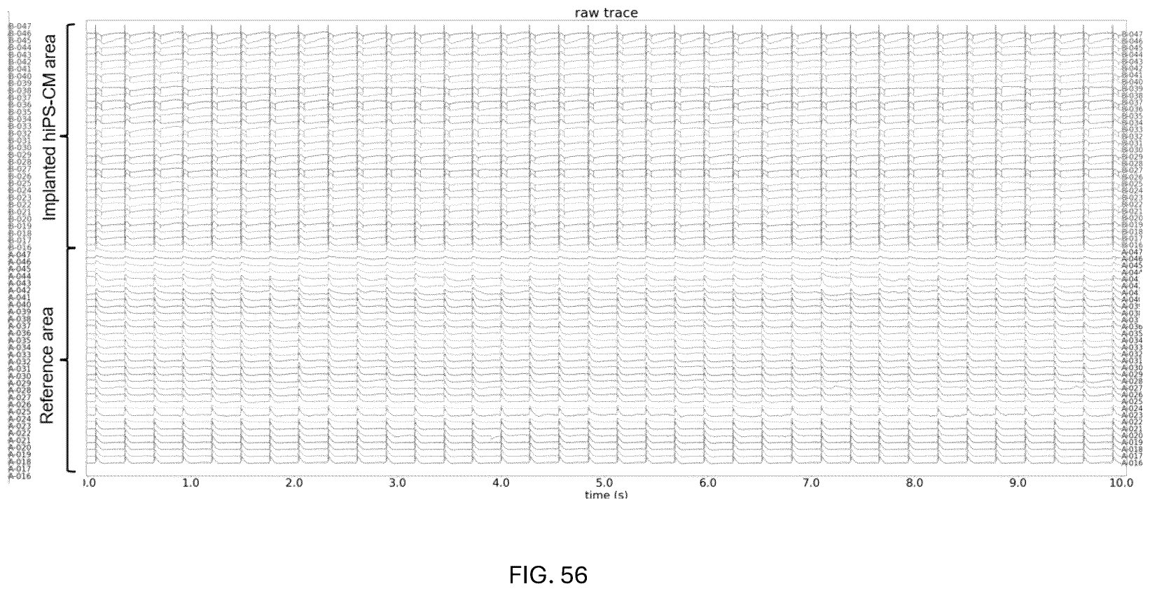

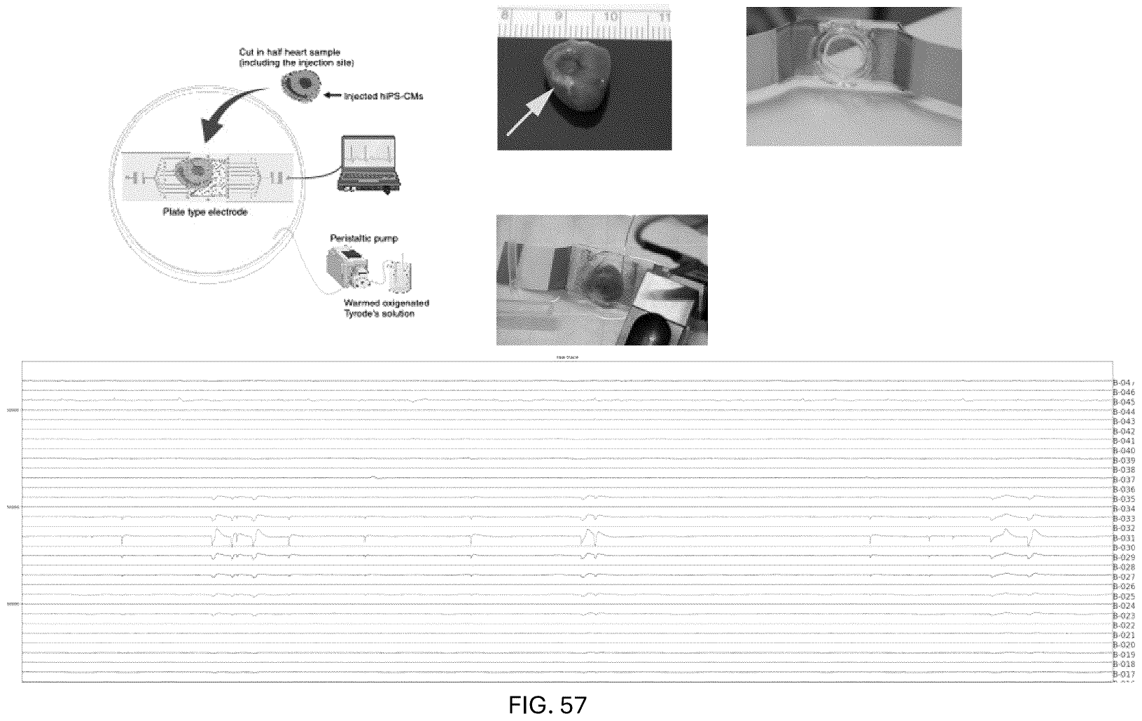

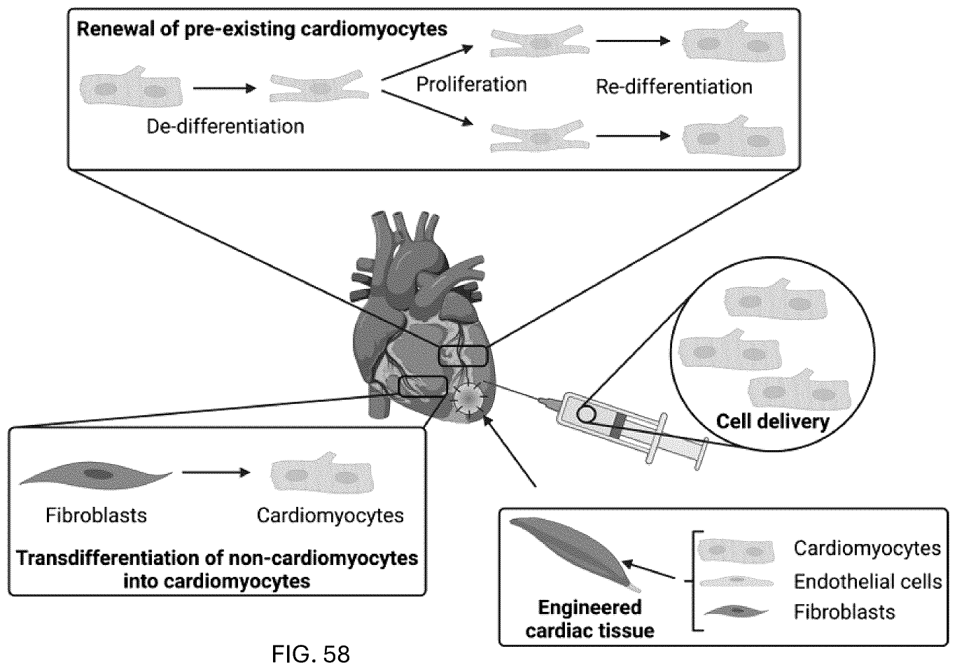

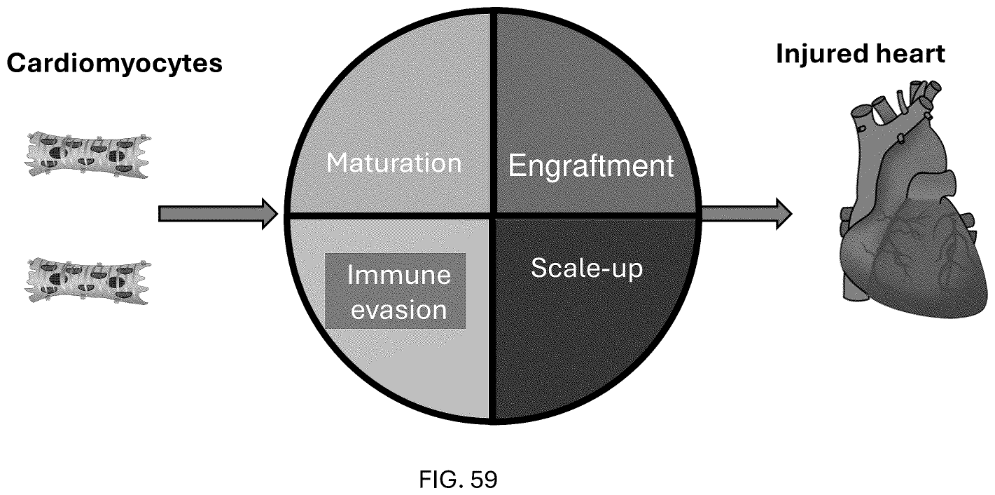

Importantly, the hiPSC-CM+RADA16 group demonstrated a more rapid reduction in the percentage of samples displaying automaticity over time compared to the hiPSC-CM Alone group (FIG. 5B, FIGS. 14-18). Analysis of inter-spike intervals (ISIs) (FIG. 19) showed much shorter ISIs of automaticity spikes in the hiPSC-CM Alone group, whereas the hiPSC-CM+RADA16 group exhibited automaticity spikes at a much slower rate, comparable to sinus spikes (FIGS. 5C-5E, FIGS. 20-25). The ratio of automaticity spike counts to sinus spike counts reduced more rapidly in the hiPSC-CM+RADA16 group, eventually reaching zero, in contrast to the hiPSC-CM Alone group (FIG. 5F). Additionally, power spectral density (PSD) analysis of raw voltage traces showed that by 135-day post-injection, the hiPSC-CM+RADA16 group exhibit electrophysiological behavior at the injection comparable to those recorded at the reference site (FIG. 5G).

### Discussion

While it is difficult and uncommon to detect arrhythmogenic automaticity in small animals (20), the use of innovative nanomesh electronics revealed transplanted hiPSC-CMs, when injected alone without RADA16, caused automaticity in rat hearts. Furthermore, electrophysiological data collected at nano-size resolution exhibited propagation of the automaticity in hiPSC-CMs to adjacent cells in rat hearts, which could lead to arrhythmias in the host heart. The comprehensive analysis of electrophysiological data from flexible mesh electronics demonstrated that co-injection with RADA16 prevents the arrhythmogenesis of hiPSC-CMs in the host heart.

Several methods, including long-time culture (21), co-culture (22, 23), electrical and mechanical stimulation (24, 25), and biochemical induction (26), have been reported to promote maturation of hiPSC-CMs (2), as evidenced by changes in gene expression and specific protein expression, as well as structural, metabolic, or cardiac function. Additionally, vascularization is a fundamental process that supplies oxygen and nutrients to transplanted hiPSC-CMs and promotes their maturation (23). While adult-like development and vascularization of hiPSC-CMs have been previously reported, the arrhythmogenic automaticity caused by transplanted hiPSC-CMs represents a key factor in future clinical translation of cardiac cell therapy. Previous studies have evaluated the vascularization of transplanted hiPSC-CMs through immunostaining with the anti-endothelial cell marker CD31 (27, 28), but the identification of CD31-positive cells as components of capillary networks does not suffice to prove functional vascularization. Here, the inventors instead directly infuse the rat endothelial marker into the host coronary arteries to demonstrate the anastomosis of neovessels in transplanted hiPSC-CMs with host vessels. This technique confirms that oxygen and nutrients were supplied to transplanted hiPSC-CMs, a requirement for engraftment, functional integration, and prolonged survival.

Previous electrophysiological studies have monitored action potentials with methods such as telemetric electrocardiogram (ECG), optical mapping, voltage mapping for the whole heart (20, 29-31), and patch clamp technique for in vitro whole-cell analysis (32, 33). The ECG approach enables the detection of automaticity and arrhythmias for the whole heart but does not allow for the detection of the origin of such signals. Optical and voltage mapping can detect the origin of arrhythmogenic automaticity and their propagation, but the resolution is low; the waveform derived from fluorescent intensity data does not preserve the signals of ECG waves.

To overcome these limitations, the inventors employed flexible mesh electronics containing 32 electrodes, each with a diameter of 25 μm and an inter-electrode spacing ranging from approximately 80 μm to 200 μm. This design enables stable and high spatiotemporal resolution detection and isolation of spike activity from hiPSC-CMs transplanted into native heart tissue, distinguishing it from native sinus rhythm at millisecond and cellular spatiotemporal resolution. Moreover, the inventors utilized a computational algorithm to differentiate different waves at millisecond resolution, enhancing the accuracy of signal interpretation.

The flexible electrodes demonstrated the capability to detect high-frequency waves that optical mapping would fail to detect. Furthermore, the voltage gradient of spikes recorded across adjacent electrodes revealed patterns of electrical propagation, allowing the inventors to predict the origin of arrhythmogenic automaticity and arrhythmias. This method provides unprecedented insights into the functional integration of hiPSC-CMs into host cardiac tissues.

In studies on hiPSC-CMs transplantation, larger animals tend to be used because the heart rates differ greatly between small animals and humans (34). Due to the refractory period, signals of automaticity caused by transplanted hiPSC-CMs may be hidden because the small animal's heart rate is much faster than that of hiPSC-CMs (20). However, due to the high resolution of electrodes used in this study, the inventors detected waves indicative of automaticity when hiPSC-CMs were injected alone. Of particular interest, the automaticity spikes at the injection site had a much higher frequency than expected or previously reported (5, 34, 35). It should be noted that the recorded high frequent spikes were only captured in the injection site (not the whole heart), which means the automaticity waves propagated in the injection site but not to all parts of the heart yet. A possible explanation is that these recorded automaticity spikes were initial arrhythmogenic spikes, such as multiple activation waves or microcircuits, that lead to major arrhythmias once these spikes are integrated.

These results show the potential of self-assembling peptide nanofiber technology for improving regenerative medicine strategies. Furthermore, these experiments show how flexible nanomesh technology can provide high resolution signals from transplanted hiPSC-CMs in the beating heart. The inventors suggest that this could provide an important safety measure for human cardiac cell therapy.

### REFERENCES

1. J. C. Garbern, R. T. Lee, Heart regeneration: 20 years of progress and renewed optimism. Dev Cell 57, 424-439 (2022).
2. P. Wu et al., Maturation strategies and limitations of induced pluripotent stem cell-derived cardiomyocytes. Biosci Rep 41, (2021).
3. E. Karbassi et al., Cardiomyocyte maturation: advances in knowledge and implications for regenerative medicine. Nat Rev Cardiol 17, 341-359 (2020).
4. Y. W. Liu et al., Human embryonic stem cell-derived cardiomyocytes restore function in infarcted hearts of non-human primates. Nat Biotechnol 36, 597-605 (2018).
5. R. Romagnuolo et al., Human Embryonic Stem Cell-Derived Cardiomyocytes Regenerate the Infarcted Pig Heart but Induce Ventricular Tachyarrhythmias. Stem Cell Reports 12, 967-981 (2019).
6. S. Marchiano et al., Gene editing to prevent ventricular arrhythmias associated with cardiomyocyte cell therapy. Cell Stem Cell 30, 741 (2023).
7. S. He et al., Advances in Injectable Hydrogel Strategies for Heart Failure Treatment. Adv Healthc Mater 12, e2300029 (2023).
8. M. H. Amer, F. Rose, K. M. Shakesheff, L. J. White, A biomaterials approach to influence stem cell fate in injectable cell-based therapies. Stem Cell Res Ther 9, 39 (2018).
9. S. Sankar et al., Clinical Use of the Self-Assembling Peptide RADA16: A Review of Current and Future Trends in Biomedicine. Front Bioeng Biotechnol 9, 679525 (2021).
10. X. Yao et al., Self-assembling peptide RADA16: a promising scaffold for tissue engineering and regenerative medicine. Nanomedicine (Lond), (2023).
11. Y. Guo, W. T. Pu, Cardiomyocyte Maturation: New Phase in Development. Circ Res 126, 1086-1106 (2020).
12. J. Wilen, J. A. Trieu, T. H. Baron, Endoscopic ultrasound-guided embolization of a gastric varix by injection of RADA16 self-assembling peptide and coils. Endoscopy 56, E266 (2024).
13. F. Branchi et al., PuraStat in gastrointestinal bleeding: results of a prospective multicentre observational pilot study. Surg Endosc 36, 2954-2961 (2022).
14. X. Liao et al., Injectable Hydrogel-Based Nanocomposites for Cardiovascular Diseases. Front Bioeng Biotechnol 8, 251 (2020).
15. J. Kurita et al., Enhanced vascularization by controlled release of platelet-rich plasma impregnated in biodegradable gelatin hydrogel. Ann Thorac Surg 92, 837-844; discussion 844 (2011).
16. S. G. Rodriques et al., Slide-seq: A scalable technology for measuring genome-wide expression at high spatial resolution. Science 363, 1463-1467 (2019).
17. M. D. Sutcliffe et al., High content analysis identifies unique morphological features of reprogrammed cardiomyocytes. Sci Rep 8, 1258 (2018).
18. R. E. Ahmed, T. Tokuyama, T. Anzai, N. Chanthra, H. Uosaki, Sarcomere maturation: function acquisition, molecular mechanism, and interplay with other organelles. Philos Trans R Soc Lond B Biol Sci 377, 20210325 (2022).
19. Z. Lin et al., Tissue-embedded stretchable nanoelectronics reveal endothelial cell-mediated electrical maturation of human 3D cardiac microtissues. Sci Adv 9, eade8513 (2023).
20. Y. Shiba et al., Human ES-cell-derived cardiomyocytes electrically couple and suppress arrhythmias in injured hearts. Nature 489, 322-325 (2012).
21. S. D. Lundy, W. Z. Zhu, M. Regnier, M. A. Laflamme, Structural and functional maturation of cardiomyocytes derived from human pluripotent stem cells. Stem Cells Dev 22, 1991-2002 (2013).
22. S. Yoshida et al., Maturation of Human Induced Pluripotent Stem Cell-Derived Cardiomyocytes by Soluble Factors from Human Mesenchymal Stem Cells. Mol Ther 26, 2681-2695 (2018).
23. H. Masumoto et al., The myocardial regenerative potential of three-dimensional engineered cardiac tissues composed of multiple human iPS cell-derived cardiovascular cell lineages. Sci Rep 6, 29933 (2016).
24. S. Yoshida, K. Sumomozawa, K. Nagamine, M. Nishizawa, Hydrogel Microchambers Integrated with Organic Electrodes for Efficient Electrical Stimulation of Human iPSC-Derived Cardiomyocytes. Macromol Biosci 19, e1900060 (2019).
25. J. L. Ruan et al., Mechanical Stress Conditioning and Electrical Stimulation Promote Contractility and Force Maturation of Induced Pluripotent Stem Cell-Derived Human Cardiac Tissue. Circulation 134, 1557-1567 (2016).
26. Y. Horikoshi et al., Fatty Acid-Treated Induced Pluripotent Stem Cell-Derived Human Cardiomyocytes Exhibit Adult Cardiomyocyte-Like Energy Metabolism Phenotypes. Cells 8, (2019).
27. H. Narita et al., Engraftment and morphological development of vascularized human iPS cell-derived 3D-cardiomyocyte tissue after xenotransplantation. Sci Rep 7, 13708 (2017).
28. J. M. Noh et al., The Activation of the LIMK/Cofilin Signaling Pathway via Extracellular Matrix-Integrin Interactions Is Critical for the Generation of Mature and Vascularized Cardiac Organoids. Cells 12, (2023).
29. J. J. Kim et al., Mechanism of automaticity in cardiomyocytes derived from human induced pluripotent stem cells. J Mol Cell Cardiol 81, 81-93 (2015).
30. W. Dhahri et al., In Vitro Matured Human Pluripotent Stem Cell-Derived Cardiomyocytes Form Grafts With Enhanced Structure and Function in Injured Hearts. Circulation 145, 1412-1426 (2022).
31. N. G. Kalkunte et al., Engineering Alignment Has Mixed Effects on Human Induced Pluripotent Stem Cell Differentiated Cardiomyocyte Maturation. Tissue Eng Part A 29, 322-332 (2023).
32. Q. Ding, X. Liu, Y. Qi, X. Yao, S. Y. Tsang, TRPAl promotes the maturation of embryonic stem cell-derived cardiomyocytes by regulating mitochondrial biogenesis and dynamics. Stem Cell Res Ther 14, 158 (2023).
33. P. Wu et al., Maturation of induced pluripotent stem cell-derived cardiomyocytes and its therapeutic effect on myocardial infarction in mouse. Bioact Mater 20, 286-305 (2023).
34. Y. Shiba et al., Allogeneic transplantation of iPS cell-derived cardiomyocytes regenerates primate hearts. Nature 538, 388-391 (2016).
35. L. Yap et al., Pluripotent stem cell-derived committed cardiac progenitors remuscularize damaged ischemic hearts and improve their function in pigs. NPJ Regen Med 8, 26 (2023).
36. X. Lian et al., Directed cardiomyocyte differentiation from human pluripotent stem cells by modulating Wnt/β-catenin signaling under fully defined conditions. Nat Protoc 8, 162-175 (2013).
37. M. A. Laflamme et al., Cardiomyocytes derived from human embryonic stem cells in pro-survival factors enhance function of infarcted rat hearts. Nat Biotechnol 25, 1015-1024 (2007).
38. P. C. Hsieh, M. E. Davis, J. Gannon, C. MacGillivray, R. T. Lee, Controlled delivery of PDGF-BB for myocardial protection using injectable self-assembling peptide nanofibers. J Clin Invest 116, 237-248 (2006).
39. X. T. Sun et al., Angiogenic synergistic effect of basic fibroblast growth factor and vascular endothelial growth factor in an in vitro quantitative microcarrier-based three-dimensional fibrin angiogenesis system. World J Gastroenterol 10, 2524-2528 (2004).
40. A. Maeda et al., Immunosuppression with FTY720 and cyclosporine A inhibits rejection of adult porcine islet xenografts in rats. Transplantation 75, 1409-1414 (2003).
41. L. Wennberg et al., Diabetic rats transplanted with adult porcine islets and immunosuppressed with cyclosporine A, mycophenolate mofetil, and leflunomide remain normoglycemic for up to 100 days. Transplantation 71, 1024-1033 (2001).
42. R. R. Stickels et al., Highly sensitive spatial transcriptomics at near-cellular resolution with Slide-seqV2. Nat Biotechnol 39, 313-319 (2021).
43. A. M. Galow et al., Quality control in scRNA-Seq can discriminate pacemaker cells: the mtRNA bias. Cell Mol Life Sci 78, 6585-6592 (2021).
44. R. Satija, J. A. Farrell, D. Gennert, A. F. Schier, A. Regev, Spatial reconstruction of single-cell gene expression data. Nat Biotechnol 33, 495-502 (2015).
45. Y. Tan et al., Silicon nanowire-induced maturation of cardiomyocytes derived from human induced pluripotent stem cells. Nano Lett 15, 2765-2772 (2015).
46. S. Zhao et al., Tracking neural activity from the same cells during the entire adult life of mice. Nat Neurosci 26, 696-710 (2023).
47. R. Liu et al., An AI-Cyborg System for Adaptive Intelligent Modulation of Organoid Maturation. bioRxiv, (2024).
48. Q. Li et al., Multimodal charting of molecular and functional cell states via in situ electro-sequencing. Cell 186, 2002-2017.e2021 (2023).

### Materials and Methods

### Human-Induced Pluripotent Stem Cell Maintenance:

This study used UCSD 142i-86-1 iPSCs (female donor, fibroblast-derived, generated by Dr. Kelly Frazer, University of California San Diego, distributed by WiCell). The Harvard University Area Institutional Review Board has reviewed this study and stated that the use of human materials (human induced pluripotent stem cells: hiPSCs) in this research does not constitute “human subjects” research. Cells were dethawed and cultured in StemFlex (Thermo Fisher Scientific, MA, USA) on Geltrex (Geltrex LDEV-Free, hESC-Qualified, Reduced Growth Factor Basement Membrane Matrix, Gibco, USA)-coated plates. StemFlex was changed every 2-3 days, and cells were passaged 1-2 times per week at 80-90% cell confluency, which was regularly monitored. When thawing cells and passaging, ROCK inhibitor (5 μM, Y-27632, Selleck Chemicals LLC, TX, USA) was utilized to promote cell survival. Cell culture supernatants were initially screened for mycoplasma contamination (IDEXX BioAnalytics, MO, USA. Case #25782-2019) and were routinely screened using the MycoStrip 50 Mycoplasma detection kit (InvivoGen, CA, USA. Catalog code: rep-mysnc-50).

Cardiomyocyte Differentiation from Human Induced Pluripotent Stem Cells

The inventors used a protocol previously published with minor modifications (36). When the hiPSCs reached ˜90% confluency, the inventors dissociated the hiPSCs from cell culture plates with a passaging reagent (ReLeSR, STEMCELL Technologies, USA), then put 150,000 cells into a 125 mL flask (Corning 125 mL Erlenmeyer Flask, USA) with 20 mL of StemScale PSC Suspension Medium (Thermo Fisher Scientific, MA, USA) (plus 5 M ROCK inhibitor for 24 hours). The flasks were put on a rotating shaker (CO₂ Resistant Shakers, cat. Number: 88881101, Thermo Scientific, USA) at 70 rounds per min. in the incubator (37 degrees Celsius, 5% CO₂). The media was replaced with StemScale without ROCK inhibitor and cells were cultured until the hiPSCs formed small embryoid bodies, at which point the inventors initiated cardiomyocyte differentiation. On day zero of differentiation, StemScale was replaced with fresh RPMI 1640 (ThermoFisher) plus B-27 supplement (ThermoFisher), 50 μg/mL ascorbic acid (AA) (RPMI/B27/AA: RBA), and 12 μM of CHIR99021 (Selleck Chemicals LLC, TX, USA). Twenty-four hours later, the media was replaced with fresh RBA without CHIR. On day 2, the medium was replaced with fresh RBA plus 5 μM of the Wnt antagonist IWP-4 (REPROCELL, Kanagawa, Japan) for 48 hours (from day 2 to 4). The media was again replaced with fresh RBA on day 4. On day 7, RBA was replaced with fresh RPMI 1640 plus B-27 supplement plus insulin at 1 μl/mL (RPMI/B27/Insulin: RBI). The RBI was changed every 2-3 days until the differentiated cardiomyocytes were used for the study, as described in the following sections.

Analysis of mRNA Expression:

At day 14 of cardiomyocyte differentiation, completion of the protocol was confirmed. Fully beating cardiomyocyte embryoid bodies were dissociated using the cardiomyocyte dissociation kit (STEMdiff Cardiomyocyte Dissociation Kit, STEMCELL Technologies, Canada) and reseeded (1.2 million cells/well) onto 6-well plates, which were prepared as follows. Each 6-well plate had 3 wells coated with Geltrex (control) and 3 wells coated with one of four self-assembling peptides (SAPs) at 0.25% concentration such that the differentiated cardiomyocytes would be cultured with the SAP. Specifically, the following SAPs were used in this study: RADA16, IEIK13, KLD12, and QLEL12. The cells were maintained for further in vitro analysis. Total RNA was extracted 7 days after initial exposure to each SAP to analyze messenger RNA (mRNA) expression of cardiomyocyte genes characteristics of an adult-like state. The inventors used Tryzol (TRIzol Reagent, Invitrogen, USA) to extract total RNA and a purification kit (E.Z.N.A. Total RNA kit I, Omega Bio-tec, Inc., Georgia, USA) according to the manufacturer's instructions. After quantification, RNA was reverse transcribed to cDNA using the High-Capacity cDNA Reverse Transcription Kit (Applied Biosystems) according to the manufacturer's instructions. Quantitative Polymerase Chain Reaction (qPCR) was performed in triplicate for each sample using the iTaq Universal SYBR Green Supermix (Bio-Rad) and the primer pairs (table S3) with a Bio-Rad CFX384 Real-Time thermal cycler. Double delta analyses were performed to compute the relative mRNA expression of each target gene using glyceraldehyde-3-phosphate dehydrogenase (GAPDH) as a housekeeping control gene.

hiPSC-CMs Preparation for In Vivo Delivery:
The differentiated hiPSC-CMs were used after confirming that the hiPSC-CMs were fully beating. The beating hiPSC-CMs were dissociated with dissociation medium (STEMdiff Cardiomyocyte Dissociation kit, STEMCELL Technologies, Canada) followed by resuspension with cardiomyocyte support medium and filtration (Cell Strainer, 40 μm Nylon, BD Falcon). Next, the inventors aliquoted the suspension medium containing 20 million hiPSC-CMs and centrifuged at 300 g for 3 minutes. Then, the inventors aspirated the supernatant and resuspended the cells in 50 μl of RBI plus pro-survival cocktails and endothelial growth factors (PVbBB): ZVAD (Z-VAD-FMK, 100 μM, Selleck Chemicals), Bcl-X_{L}. BH4 (Bcl-x_{L} BH44-23, Human, Cell-Permeable, 50 nM, Calbiochem), Cyclosporine A (200 nM, Wako Pure Chemicals), IGF-1 (Recombinant Human IGF-I, 100 ng/ml, PEPROTECH, USA), and Pinacidil (50 μM, Sigma) (37). In addition, the inventors added VEGF (Recombinant Human VEGF165, 50 ng/mL, PEPROTECH, USA), bFGF (Recombinant Human FGF basic/FGF2/bFGF Protein, 50 ng/mL, R&D System), PDGF-BB (Recombinant Human PDGF-BB, 1 μg/mL, PEPROTECH, USA) as RBI-PVbBB (38, 39). The cell suspension was kept on ice until the injection. Right before injection, additional RBI-PVbBB at 50 μl was added to the hiPSC-CMs suspension, and 50 μl of 0.5% RADA16 adjusted with 10% sucrose water-PVbBB was added to hiPSC-CMs with RADA16 suspension (both hiPSC-CMs at two hundred million cells/mL).

### Animals and Ethics Statement:

Eight-week-old RNU Nude rats were purchased from Charles River Laboratory (MA, USA) and held in the designated, pathogen-free animal facility of Harvard University. All animal experiments were performed according to the Guide for the Use and Care of Laboratory Animals and approved by the Institutional Animal Care and Use Committee (IACUC) of Harvard University Faculty of Arts and Sciences (protocol ID: 17-01-286-2).

### Ultrasound-Guided Cell Injection:

The inventors used RNU nude rats (8-10 weeks old, male, 250-350 g). Rats received cyclosporine injections (15 mg/kg) as an immunosuppressant a day before the injection and every day after surgery (40, 41). The rats were numbered and randomly divided into two groups. They were sedated using Isoflurane (initially at 3%, maintenance at 1-2%), secured to an electrode-heating platform, and had chest hair removed using depilatory cream. A long-axis heart image was obtained with an ultrasound transducer (Vevo 3100, FUJIFILM, Japan) applied to the chest. Using a syringe and 30-gauge needle attached to a mount/injection guide, 150 μL of cell suspension (hiPSC-CMs suspension or hiPSC-CMs with RADA16 suspension randomly) prepared in advance was injected (ultrasound-guided) into the anterior wall of the left ventricle for immunostaining (with six independent animals for each group), cell retention analysis (with four independent animals for each group), and Slide-seq (with five independent animals for each group). Additionally, the inventors injected the cell suspension around the pericardium and into the left ventricle wall for the electrophysiological study (with three to four independent animals for each group). After ultrasound-guided intramyocardial injection, rats were returned to their cages and allowed to recover. Cyclosporine (15 mg/kg) was administered intraperitoneally on a daily basis to suppress immune reactions until the heart was harvested.

### Immunofluorescence Staining:

The rats were euthanized on each applicable day (day 7 and day 84) with overdose (5%) carbon dioxide inhalation. After verifying the euthanasia, the chest wall was opened, followed by an infusion of heparin-PBS (Heparin sodium salt from porcine intestinal mucosa, CAS Number: H3393, Sigma-Aldrich, USA. 1000 U/kg) from the inferior vena cava (IVC) and right and left ventricle (LV) chamber. Then, the distal ascending aorta was ligated with a 4-0 silk suture, followed by heparin infusion from the proximal aorta to the coronary arteries directly with the right atrium (RA) open to infuse efficiently with decreased RA pressure. Next, the heart was fixed by incubating fresh 4% paraformaldehyde (PFA: PARAFORMALDEHYDE 16% SOLUTION, EM GRAFE, Electron Microscopy Sciences, USA) in PBS infused from the coronary arteries. After harvesting the heart, the inventors cut it into two pieces horizontally through the injection site and placed it in 10% sucrose (S9378, lot #SLBL4342V, Sigma) in PBS until it sank and then 20% sucrose in PBS overnight. The samples were embedded in an O.C.T. Compound (Tissue-Tec, SAKURA), put on dry ice for cryopreservation, and sectioned with the Cryostat (Leica CM 1950) at 18 μm.

For the samples stained with GSL I (Griffonia Simplicifolia Lectin I (GSL I) Isolectin B4, DyLight 649 (DL-1208-. 5)), the rats were euthanized on day 28 with an overdose of inhalated carbon dioxide (5%). After verifying euthanasia, the chest wall was opened, followed by an infusion of heparin (1000 U/kg) from the IVC and apex to the LV chamber. Then, the distal ascending aorta was ligated, followed by heparin infusion from the proximal aorta to the coronary arteries directly, with the RA open to infusion efficiently with decreased RA pressure. Then, the heart was fixed by incubation in fresh 4% PFA. The GSL I solution (50 μg/mL) was infused into the coronary arteries and then incubated for two hours at room temperature. After harvesting the heart, the inventors cut it into two pieces horizontally through the injection site and placed it in 10% sucrose in PBS until it sank and then 20% sucrose in PBS overnight. Then, the samples were embedded in an O.C.T. Compound, put on dry ice for cryopreservation, and sectioned with the Cryostat at 18 μm.

Immunostaining was performed for the cryosections. Briefly, the sections were permeabilized with 0.05% Tween 20 (Tween 20 Reagent Grade CAS #9005-64-5, VWR LIFE SCIENCE, USA) for 10 min. and 0.3% Triton X (Triton X-100, for molecular biology, Sigma) twice for 10 min., followed by 5% goat serum-PBS blocking for an hour at room temperature. Then, each sample was incubated overnight at four degrees Celsius with the primary antibody solution diluted with 1% goat serum-PBS. The primary antibodies were selected for each sample depending on the target proteins: anti-cardiac troponin T antibody (Alexa Fluor 647 Mouse Anti-Cardiac Troponin T (cat #565744): cTnT, BD Pharmagen. 1:200), anti-human mitochondria antibody (Anti-Mitochondria antibody [113-1]-BSA and Azide free (ab92824): hMito, Abcam. 6 μg/mL), anti-CD31 antibody (CD31/PECAM-1 Antibody (NB 100-2284): CD31, NOVUS BIOLOGICALS. 1:250), and anti-human troponin I antibody (Recombinant Anti-Cardiac Troponin I antibody [EP1106Y] (ab52862): hTnI, Abcam 1:250). The next day, the samples were washed with 0.05% Tween-20 for 10 min. three times, followed by incubation with secondary antibodies (Goat anti-Mouse IgG (H+L) Highly Cross-Adsorbed Secondary Antibody, Alexa Fluor Plus 555 (A32727) 1:1000, Thermo Fisher Scientific, MA, USA, Goat anti-Rabbit IgG (H+L) Cross-Adsorbed Secondary Antibody, Alexa Fluor 488 (A11008) 1:100 Thermo Fisher Scientific, MA, USA) diluted in 1% goat serum-PBS for an hour at room temperature. After washing the samples with 0.05% Tween-20, the samples were incubated with autofluorescence quenching regent (Vector True VIEW Autofluorescence Quenching Kit (SP-8400-15), vector LABORATORIES, USA) for six minutes. at room temperature. Then, the samples were shielded with a mounting medium (VECTASHIELD PLUS Antifade Mounting Medium with DAPI, vector LABORATORIES, USA) and observed with a confocal microscope (Zeiss LSM 880 Confocal Laser Scanning Microscope, Zeiss).

### Injected Cardiomyocytes and Vascularization Area

Immunofluorescence staining was performed for the cryosectioned samples on day 7 (n=6), day 28 (n=4), and day 84 (n=4) after injection. At least four representative sections were selected randomly from different levels of the injection area for each sample. A confocal microscope (Zeiss LSM 880 Confocal Laser Scanning Microscope, Zeiss) was used to observe the target objectives in each pseudo color. Each sliced sample was randomly assigned a number corresponding to its respective group, ensuring an unbiased selection process. Additionally, the samples were blinded, meaning that their group assignments remained concealed throughout the analysis to minimize potential biases and enhance the reliability of the results. The inventors used the latest available version of ImageJ Fiji to analyze the immunofluorescence images. Then, the area of injected hiPSC-CMs and vessels, positive for each specific antibody, was calculated with ImageJ, followed by obtaining the average of each area across sections. Regarding the hiPSC-CMs injected area analysis in FIG. 1D, the images of the double-positive cardiomyocytes for hMito and cTnT antibodies surrounded by the area with only DAPI-positive staining were first converted to black and white images. The pixels of the area were calculated by ImageJ and converted to millimeters square for each image according to the image size from the image software (ZEN blue edition and Lite, Zeiss). The area of the vessels (CD31 positive area or GSL I positive area) in the injected hiPSC-CM area was calculated by extracting the area of the injected hiPSC-CMs, then splitting the channel color and calculating the pixels of the vessel's area, converted into millimeters squared. The vessel area (CD31 positive area or GSL I area) ratio in the injected hiPSC-CMs area (cTnT or hMito positive area) was calculated according to the area calculated above (FIGS. 1E, 1G and FIG. 3H).

### Analysis of Cell Retention in the Left Ventricular Wall

Two weeks before imaging started, the rats were put on an alfalfa-free dict (5V5R-PicoLab Select Rodent 50 IF/6F, LabDiet, USA) to avoid autofluorescence from rat organs on the recording. The differentiated and beating hiPSC-CMs were dissociated with a dissociation medium and aliquoted at 20 million cells per sample, followed by centrifugation at 300 G for three minutes. Then, the cell pellet was resuspended with PBS and mixed with a near-infrared fluorescent lipophilic cell labeling dye (Vivo Track 680, IVISense 680 Fluorescent Cell Labeling Dye, NEV12000, Lot: 3095940, PerkinElmer, USA) according to the manufacturer's instructions. After labeling the cells with Vivo Track 680, cells were resuspended either with 100 μl of RBI-PVbBB as hiPSC-CM Alone group or 50 μl of RBI-PVbBB plus 50 μl of 0.5% RADA16 adjusted with 10% sucrose water-PVbBB as hiPSC-CM+RADA16 group, and 100 μl of RBI-PVbBB as a control group. Then, the labeled hiPSC-CMs suspensions were injected (ultrasound-guided) into the rat's anterior left ventricular wall. After day 7 of injection, the rats were sedated with isoflurane in the imaging chamber of the IVIS Spectrum in vivo imaging system (PerkinElmer Inc., USA), after which images were taken of the retained labeled hiPSC-CMs. The image was analyzed with the software (Living Image). For both in vivo samples and ex vivo samples from harvested hearts, the “Max Radiant Efficiency” value of the images was analyzed.

### Spatial Transcriptomics Analysis

Each rat was euthanized with an overdose of carbon dioxide (5%) on day 7 or 21 after the cell suspension injection, after which the chest wall was opened. Then, heparin-PBS solution (1000 U/kg) was injected into the inferior vena cava, right atrium, right ventricle, left ventricle, and coronary arteries to wash out blood cells (the same procedure performed when harvesting the heart for immunofluorescence staining). Unfixed rat hearts were frozen in liquid nitrogen immediately after harvest and sliced at a thickness of 10 μm. Sections were attached to pucks for Slide-seq analysis. Gene expression was analyzed with rat and human reference libraries.

### 1. Generation of Slide-Seq Transcriptomics Data

Slide-seq samples were prepared following the Slide-seqV2 protocol (42) derived from the original Slide-seq protocol (16). A 10 μm section was cut and the puck was adhered to the section to include the hiPSC-CMs injected zone. RNA hybridization and first strand synthesis were subsequently performed prior to tissue digestion and isolation of the beads. Subsequently, second strand synthesis was performed following Exol treatment and wash. PCR libraries were then generated following the Slide-seqV2 protocol before cleanup, tagmentation, and quantification via Bioanalyzer. A shallow sequencing run was initially performed on a Nextseq 1000 platform at 30 million reads per sample to assess tagmentation quality of libraries before a final deeper sequencing run is performed on a Novaseq600 platform (full sequencing metadata are found in table S4). The reads from the shallow and deep sequencing runs were subsequently pooled and mapped against the array barcodes per library. In total, 15 libraries were prepared per heart (n=3 for hiPSC-CM Alone harvested on day 7, n=5 for hiPSC-CM Alone harvested on day 21, n=4 for hiPSC-CM+RADA16 rats harvested on day 7, and n=4 for hiPSC-CM+RADA16 rats harvested on day 21). The raw sequencing data was then processed as described in the Slide-seqV2 protocol to align reads and encode spatial data for further processing.

### 2. Analysis of Slide-Seq Data

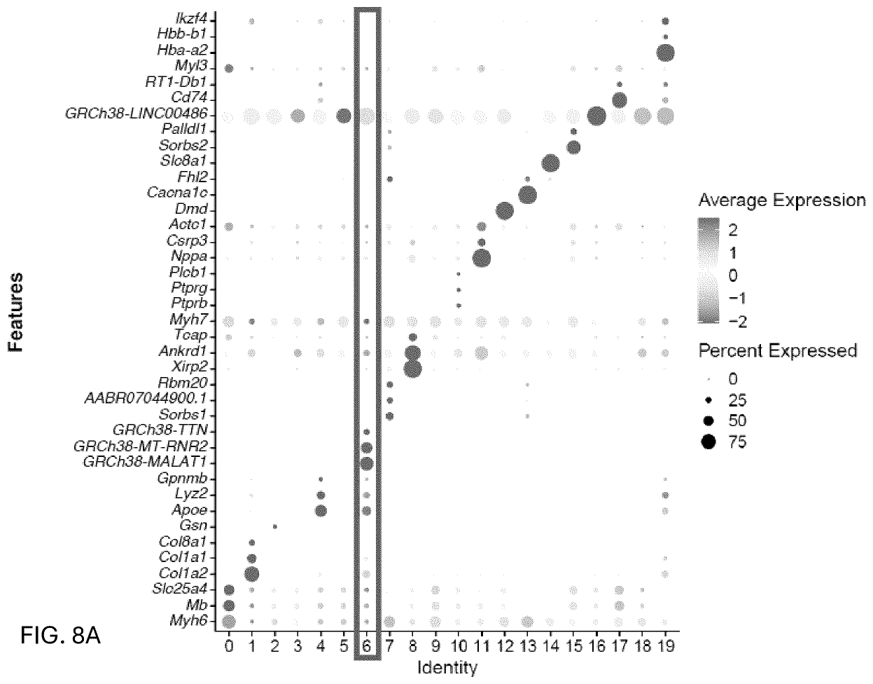

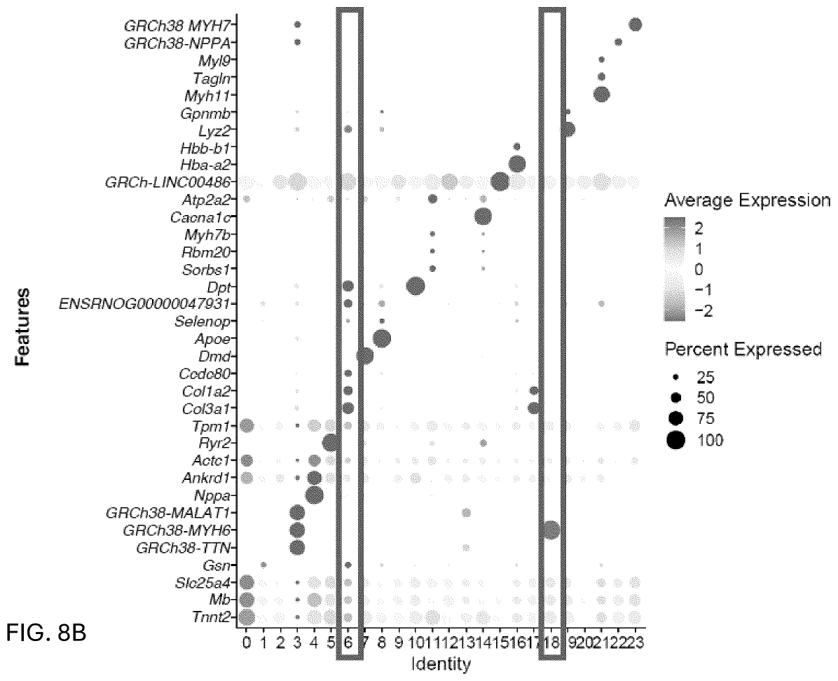

Sequenced libraries from slide-seq samples were loaded into the Seurat package in R, preserving both single-cell and spatial resolution. The data were filtered and normalized using the regularized negative binomial regression via the SCTransform function with 3000 cells used for subsampling to construct the negative binomial distribution. The data were not filtered by mitochondrial transcripts due to the documented relative abundance of mitochondrial activity in cardiac tissue to non-cardiac tissue (43). Dimensionality reduction and clustering were then performed using the standard Seurat spatial workflow for further analysis (44). Specifically, the inventors then performed dimensionality reduction via PCA and UMAP (utilizing the first 30 dimensions) before clustering via the FindNeighbors (on the first 30 dimensions) and FindClusters (with resolution of 0.5) in Seurat. When performing comparisons within harvest time (i.e. hiPSC-CM+RADA16 vs hiPSC-CM Alone at either 7-days or 21-days), only the compared datasets were analyzed and clustered together with hiPSC-CMs being identified by human titin (TTN) and other cardiomyocyte marker gene expression (FIG. 8).

To isolate hiPSC-CMs from rat cardiac cells, the inventors filtered all beads to focus on those expressing human TTN due to its role as a general cardiomyocyte marker and aggregated them by treatment and timepoint. Differential gene analysis was conducted in Seurat with this subset of cells to characterize the effects of RADA16 treatment on gene expression at different time points following cell suspension injection. Specifically, the inventors examined shifts in the expression of gene isoforms involved in myofibril assembly associated with adult-like cardiomyocytes (MYH6 to MYH7, TNNI1 to TNNI3, and MYL7 to MYL2) and changes in gap junction formation and ion channel expression (i.e., decreased automaticity ion channels, increased ventricular ion channels, calcium ion handling, and gap junction formation) (11). Differential gene expression analysis was performed utilizing the FindMarkers function in Seurat which utilizes a Wilcoxon Rank Sum test with Bonferroni multiple-comparison correction. Significantly differentially expressed genes were defined as those with an absolute value log 2 fold change greater than 2 and an adjusted p-value less than 0.05.

### Analysis of Sarcomere Organization

The inventors performed immunofluorescence staining for rat heart cryosections taken on day 7 and day 84 after cell suspension injections (hiPSC-CM Alone or hiPSC-CM+RADA16). The sections were stained with anti-cardiac troponin T antibody (Alexa Fluor 647 Mouse Anti-Cardiac Troponin T (cat #565744): cTnT, BD Pharmagen. 1:200), anti-human troponin I antibody (Recombinant Anti-Cardiac Troponin I antibody [EP1106Y] (ab52862): hTnI, Abcam 1:250), and DAPI as performed in the immunofluorescent staining. Then, the stained sections were observed with a confocal microscope (Zeiss LSM 880 Confocal Laser Scanning Microscope, Zeiss) using an oil immersion objective at 64× for the sarcomere organization analysis. In this study, the inventors applied SarcOmere Texture Analysis (SOTA) to quantify sarcomere organization (17). SOTA was conducted by independent investigators who had no prior knowledge of the sample information. This methodological approach minimized potential biases and enhanced the reliability of the results.

### 1. Segmentation of Myofilament Bundles

hiPSC-CMs were stained with hTnI to identify myofilaments. The inventors then manually segmented the largest myofibril bundle in twenty cells per image, selected at random. Myofibril bundles were identified as chains of laterally connected myofibrils with contiguous sarcomere orientation. Myofibril bundles with insufficient mean intensity for meaningful sarcomere analysis (<0.23, based on histogram across all samples) were excluded.

### 2. Sarcomere Length Measurement

Sarcomere length was measured as the distance between hTnI-stained Z-lines from cardiomyocytes with visible sarcomere structures. An average of 10 sarcomeres from each sample was calculated on an interpolated YZ projection to account for spatial distribution across different regions of the image (45). This manual image analysis was performed in ImageJ Fiji.

### 3. Sarcomere Organization Score Assessment

The inventors performed the automated analysis of sarcomere organization using SOTA, which was previously validated in endogenous, direct reprogrammed, and induced pluripotent-derived cardiomyocytes (17). SOTA calculates sarcomere organization using Haralick texture features, using correlations in pixel intensity correlations from 1 to 40-pixel offsets and angles from 0 to 180 degrees. From the resulting m×n Haralick correlation matrix, calculates the maximum peak prominence across all angles, resulting in the SOTA sarcomere organization score. Peaks at pixel offset >15 were excluded, as they did not exhibit the signature SOTA decaying sinusoidal pattern of verified sarcomeres. The inventors also calculated the primary sarcomere orientation using the SOTA angle of peak prominence. To assess the deviation in intercellular alignment of sarcomere orientation, the inventors calculated the difference from the mean orientation of analyzed myofibril bundles in that image.

### 4. Statistics

Analysis and final plots were conducted in R-studio and MATLAB. The number of samples and statistical analysis methods are stated in the figure legend. For sarcomere organization score analysis via SOTA, one-way ANOVA, followed by Games-Howell's post hoc test were performed. Similarly for sarcomere length analysis, two-way ANOVA with Tukey's multiple comparison post hoc test were performed where ns=not significant, *<0.05, **<0.01, ***<0.001, and <0.0001. The deviation in the intercellular alignment of sarcomere orientation was calculated via the difference from the mean orientation. The inventors excluded the samples where sarcomere orientation=zero. Bartlett's test was performed to compare variances or standard deviations from the control and RADA16-treated groups.

Electrophysiological Study with Flexible Mesh Nanoelectronics

### Fabrication of Flexible Mesh Nanoelectronics

The fabrication of flexible mesh nanoelectronics followed established protocols (46-48). A 4-inch SiO2/Si wafer was used as the substrate. It was cleaned with piranha solution, thoroughly rinsed with deionized (DI) water, and dried using nitrogen. To enhance adhesion, hexamethyldisilazane (HMDS) was spin-coated at 4000 rpm. A bilayer of LOR 3A (300 nm) and S1805 (500 nm) photoresists was then applied, followed by baking at 180° C. for 5 minutes and 115° C. for 1 minute. Nickel patterns were defined by exposing the substrate to ultraviolet (UV) light at 40 mJ/cm² using a mask aligner, and the patterns were developed with CD-26. Residues were removed with oxygen plasma treatment. A 100-nm nickel layer was deposited using thermal evaporation, followed by a lift-off process in remover PG. To create the encapsulation layer, SU-8 2002 was spin-coated at 4000 rpm and pre-baked at 65° C. and 95° C. for 2 minutes each. The layer was exposed to UV light at 200 mJ/cm2, post-baked under the same conditions, and developed with SU-8 developer. After rinsing with isopropyl alcohol (IPA) and drying with nitrogen, the encapsulated layer was hard baked at 180° C. for 40 minutes. Interconnect patterns were then defined using the same bilayer photoresist process, followed by deposition of 5/40/5 nm chromium/gold/chromium layers using an electron-beam evaporator and another lift-off procedure. Electrode arrays were patterned similarly, with 5/50 nm chromium/platinum deposited via electron-beam evaporation. Finally, a top SU-8 encapsulating layer was fabricated using the same method as for the bottom layer.

### Packaging of Flexible Mesh Nanoelectronics

The packaging process began with soldering a flexible flat cable (Molex) onto the input/output pads using a flip-chip bonder. Then, a biocompatible adhesive (Kwik-Sil) was used to fully enclose the device. Platinum black was electroplated onto the platinum electrodes to enhance conductivity. The entire device was rinsed with DI water for 30 seconds and dried with nitrogen.

To release the device, the surface was treated with light oxygen plasma, and nickel etchant (type TFB) was used to fully release the device from the substrate. After release, the device was prepared for sterilization under UV light before using it for recording.

Transplantation of iPSC-CMs with or without RADA16 in Rats

The inventors used RNU nude rats (8-10 weeks old, male, 250-350 g). Rats received cyclosporine injections (15 mg/kg) as an immunosuppressant a day before the injection and every day after surgery. The 150 μL of cell suspension (hiPSC-CMs with/without 0.25% RADA16 in PVbBB, hiPSC-CMs at two hundred million cells/mL), or as control; 150 μL of saline (SHAM), 150 μL of 0.25% RADA16-PVbBB (RADA16), or 150 μL of human fibroblast (at two hundred million cells/mL)—PVbBB suspension was injected around the pericardium and into the left ventricle wall via an ultrasound guide.

Electrophysiological Recording with Flexible Mesh Nanoelectronics

After at least five days of observation in healthy conditions, the rats were applied to the electrophysiological study. Considering the required period for the expression of ion channels, decrease in pacemaker cells, and formation of electrophysiological junctions between transplanted hiPSC-CMs and host cardiomyocytes, the electrophysiological study was done at the following time points: approximately two weeks, 30 days (27-31 days), 80 days (77-88 days), and 135-160 days. As a pre-surgical analgesic, buprenorphine (0.1 mg/kg) was administered to the rats before anesthesia. The rat was anesthetized with 5% isoflurane (inhalation), after which the chest wall was opened to infuse heparin solution (1000 U/kg) from the inferior vena cava, right ventricle, and left ventricle. The distal ascending aorta was ligated with a 4-0 silk suture, followed by infusion of heparin to coronary arteries, with the right atrium cut open to infuse efficiently with decreased right atrium pressure. Then, the heart was harvested, and the ascending aorta was connected to an aortic cannula (Aortic Cannula for Rat Heart to IH-SR, OD 2.3 mm. Item number: 73-2814, Harvard Apparatus, USA). The oxygenated (95% Oxygen and 5% Carbon dioxide) Tyrodes' solution (in mM, 128.2 NaCl (Sodium chloride for biotechnology, CAS Number: 7647-14-5, VWR, USA), 1.3 CaCl2 (Calcium chloride, Product number: C1016, Sigma-Aldrich, USA), 4.7 KCl (Potassium chloride, Product number: P9333, Sigma-Aldrich, USA), 1.19 NaH2PO4 (Sodium Phosphate, Cat #: P-907, Boston BioProducts, USA), 1.05 MgCl2 (Magnesium chloride, Product number: M8266, Sigma-Aldrich, USA), 20.0 NaHCO₃(Sodium bicarbonate, Product number: S5761, Sigma-Aldrich, USA), and 11.1 glucose (D-(+)-Glucose, Product number: G7021, Sigma-Aldrich, USA)) was perfused from the cannula for pre-circulation, after which the heart was connected to a Langendorff system (IH-SR Langendorff, Harvard Apparatus, USA). Pre-warmed oxygenated Tyrode's (32 degrees Celsius with a heating circulator (Thermo Haake C10-B3 Recirculating Water Bath, ARTISAN TECHNOLOGY GROUP, USA)) solution was perfused at approximately 9 mL/min with a peristaltic pump (Reglo Digital 4 Cassette Dispensing Pump, ISM597D, ISMATEC). The temperature of the circulated solution, the chamber of the Langendorff, and the heart rate were monitored with a data acquisition system (Bridge Amp (PowerLab 8/30, ADInstruments, USA), Bio Amp (Product code: FE231, ADInstruments, USA), Spring Clip Electrodes (Product code: MLA1214, AD ADInstruments, USA), and LabChart software v8.1.20) until the heart beating became stable. Once stable heart beating was confirmed, flexible mesh nanoelectronics were attached to the injection site on the left anterior ventricular wall (experiment area) and the right ventricular wall (reference site). The Langendorff technique (isolated heart perfusion) was utilized to record the action potentials of each rat heart sample. Samples were excluded from the analysis if spontaneous beating ceased within the first ten minutes of recording. These groups originally numbered 4 animals for each group but some ceased spontaneous beating during the recording, so had to be removed from the study.

### Electrophysiological Data Analysis

Independent investigators blinded to the sample information conducted recording and electrophysiological analysis. This approach minimized potential biases and improved the reliability and objectivity of the results. The inventors analyzed the electrophysiological data with a customized Python script. The inventors first preprocessed raw voltage traces by applying “bandpass_filter” to remove low and high-frequency noises via SpikeInterface package. The inventors then detected electrical spikes using “find_peaks” from Scipy. The inventors manually curated the detected spikes into host sinus rhythm, automaticity/arrhythmia rhythm, and noises.

Spikes were classified as host sinus rhythm based on the following criteria:
1. The spikes were recorded at both the injection and reference sites.
2. The spikes exhibit distinguishable QRS waves.

Spikes were classified as automaticity/arrhythmia rhythm based on the following criteria:
1. The spikes were observed exclusively from the recording channels at the injection site.
2. The spikes appeared on a smooth baseline.
3. The spikes occurred multiple times.
4. The spikes were captured within a biologically plausible propagation window by at least two adjacent channels.

Spikes were classified as noise based on the following criteria:
1. Noise spikes were sometimes captured at both injection and reference sites.
2. Noise spikes were irregular in electrical features such as frequency, waveform, etc.
3. Noise spikes sometimes manifested as small voltage fluctuations on top of baseline traces with low-frequency noise.

The inventors then computed the inter-spike intervals (ISIs) for the classified host heartbeat rhythm and automaticity/arrhythmia rhythm, respectively (FIG. 19). To compare ISI distribution across subjects, the inventors normalized the host heartbeat ISIs and automaticity ISIs collectively for each subject. Specifically, the inventors used the largest host heartbeat ISI as the denominator, such that all host heartbeat ISIs fall into the range 0-1 after normalization. Automaticity ISIs are divided by the same denominators, and their normalized values can exceed 1.

The inventors applied Welch's method with Scipy to conduct the power spectral density (PSD) analysis. The inventors applied the “find_peaks” method to extract the spike activation time and peak amplitudes. To smooth the detected value to compensate for losses caused by noises, the inventors averaged the values from adjacent channels to fill missing positions.

TABLE S1
Full list of differentially expressed genes

| | | | | | |
|---|---|---|---|---|---|
| Gene | p_val | avg_log2FC | pct. 1 | pct. 2 | p_val_adj |

Day 7 CM alone vs CM + RADA16

| | | | | | |
|---|---|---|---|---|---|
| GRCh38-------MYH7 | 1.14E−195 | 3.596626577 | 0.419 | 0.059 | 3.33E−191 |
| GRCh38-------MYL2 | 2.74E−183 | 5.190019445 | 0.324 | 0.012 | 8.02E−179 |
| GRCh38-------MT-RNR2 | 9.20E−152 | 2.264944436 | 0.688 | 0.504 | 2.69E−147 |
| GRCh38-------COL3A1 | 1.05E−135 | −2.874875694 | 0.047 | 0.307 | 3.06E−131 |
| GRCh38-------MT-RNR1 | 1.25E−128 | 2.324847696 | 0.493 | 0.202 | 3.64E−124 |
| GRCh38-------MYL3 | 8.01E−119 | 2.473977784 | 0.335 | 0.07 | 2.34E−114 |
| GRCh38-------COL1A2 | 1.97E−91 | −2.19297118 | 0.053 | 0.258 | 5.75E−87 |
| GRCh38-------NPPB | 3.31E−82 | 2.515710171 | 0.22 | 0.035 | 9.69E−78 |
| GRCh38-------C7 | 5.37E−80 | −3.301559316 | 0.019 | 0.172 | 1.57E−75 |
| Rattus-norvegicus-Cd74 | 8.78E−77 | −2.875056743 | 0.029 | 0.187 | 2.57E−72 |
| GRCh38-------CCDC80 | 1.77E−66 | −5.007630902 | 0.004 | 0.115 | 5.18E−62 |
| GRCh38-------FN1 | 1.18E−64 | −2.308497382 | 0.044 | 0.196 | 3.45E−60 |
| GRCh38-------EMC10 | 1.10E−54 | 2.156417533 | 0.212 | 0.062 | 3.21E−50 |
| Rattus-norvegicus-RT1-Db1 | 8.99E−53 | −3.336052759 | 0.012 | 0.114 | 2.63E−48 |
| GRCh38-------MYLK | 2.15E−52 | −3.805138038 | 0.008 | 0.104 | 6.29E−48 |
| GRCh38-------COL14A1 | 2.19E−48 | −5.343233934 | 0.002 | 0.081 | 6.39E−44 |
| GRCh38-------ITGA8 | 4.58E−42 | −6.099129257 | 0.001 | 0.068 | 1.34E−37 |
| GRCh38-------MYH11 | 8.68E−42 | −4.2446701 | 0.003 | 0.075 | 2.54E−37 |
| GRCh38-------MT-ND6 | 1.51E−41 | 2.557523117 | 0.123 | 0.022 | 4.42E−37 |
| GRCh38-------SORBS2 | 2.00E−41 | 2.450274862 | 0.138 | 0.031 | 5.85E−37 |
| GRCh38-------FBN2 | 3.72E−34 | 2.636690598 | 0.105 | 0.02 | 1.09E−29 |
| GRCh38-------FHL2 | 8.61E−33 | 2.54347878 | 0.101 | 0.02 | 2.52E−28 |
| GRCh38-------AC015522.1 | 6.79E−31 | 7.500163738 | 0.055 | 0 | 1.98E−26 |
| GRCh38-------PRDX1 | 9.93E−30 | −2.366786317 | 0.016 | 0.084 | 2.90E−25 |
| GRCh38-------NFIA | 2.34E−29 | −2.765060599 | 0.012 | 0.075 | 6.85E−25 |
| GRCh38-------ENO3 | 4.69E−29 | 2.146314169 | 0.102 | 0.024 | 1.37E−24 |
| GRCh38-------MB | 1.10E−28 | 2.82038349 | 0.075 | 0.01 | 3.20E−24 |
| GRCh38-------SULF1 | 1.98E−28 | −5.08291903 | 0.001 | 0.048 | 5.78E−24 |
| GRCh38-------FABP3 | 5.29E−28 | 2.192041442 | 0.094 | 0.021 | 1.55E−23 |
| Rattus-norvegicus-RT1-Ba | 1.64E−27 | −2.998642119 | 0.007 | 0.06 | 4.78E−23 |
| GRCh38-------MAGI2 | 1.92E−27 | −2.14033344 | 0.024 | 0.095 | 5.62E−23 |
| GRCh38-------COL6A1 | 1.52E−25 | −2.464979207 | 0.013 | 0.07 | 4.45E−21 |
| Rattus-norvegicus-RT1-Da | 2.70E−25 | −2.84413217 | 0.008 | 0.059 | 7.90E−21 |
| GRCh38-------RBP1 | 1.01E−24 | −2.247581695 | 0.016 | 0.075 | 2.95E−20 |
| GRCh38-------PRKG1 | 6.50E−24 | −2.138875436 | 0.016 | 0.073 | 1.90E−19 |
| GRCh38-------FBN1 | 4.99E−23 | −2.922742005 | 0.007 | 0.053 | 1.46E−18 |
| GRCh38-------LINC01411 | 4.12E−22 | 4.66479444 | 0.045 | 0.003 | 1.20E−17 |
| GRCh38-------MYO18B | 1.51E−21 | 2.160471363 | 0.085 | 0.023 | 4.41E−17 |
| GRCh38-------MASP1 | 2.37E−21 | 3.06651056 | 0.053 | 0.006 | 6.94E−17 |
| GRCh38-------LSAMP | 3.73E−19 | −2.2446701 | 0.008 | 0.049 | 1.09E−14 |
| GRCh38-------IGFBP5 | 2.15E−18 | 2.42854081 | 0.056 | 0.011 | 6.28E−14 |
| GRCh38-------MFAP4 | 3.29E−18 | −2.522204075 | 0.008 | 0.047 | 9.60E−14 |
| GRCh38-------MID1 | 3.11E−17 | 2.308273405 | 0.083 | 0.028 | 9.10E−13 |
| GRCh38-------CKM | 8.29E−17 | 2.66322202 | 0.046 | 0.007 | 2.42E−12 |
| GRCh38-------PDE3A | 9.53E−17 | −2.510131243 | 0.007 | 0.043 | 2.79E−12 |
| GRCh38-------PRSS35 | 2.73E−16 | 5.174763451 | 0.029 | 0 | 7.99E−12 |
| GRCh38-------AQP3 | 7.32E−16 | −6.107166576 | 0 | 0.024 | 2.14E−11 |
| GRCh38-------ROR1 | 1.58E−15 | 2.19083568 | 0.066 | 0.02 | 4.63E−11 |
| GRCh38-------ACTA1 | 1.88E−15 | 2.259155638 | 0.053 | 0.012 | 5.50E−11 |
| Rattus-norvegicus-C3 | 2.44E−15 | −3.744596497 | 0.001 | 0.027 | 7.14E−11 |
| GRCh38-------PCDH7 | 3.60E−15 | 2.578367645 | 0.051 | 0.011 | 1.05E−10 |
| Rattus-norvegicus-Penk | 5.05E−15 | 2.767543614 | 0.041 | 0.007 | 1.48E−10 |
| GRCh38-------LRRC17 | 2.37E−14 | −2.821764357 | 0.004 | 0.033 | 6.92E−10 |
| GRCh38-------ERBB4 | 4.32E−14 | 2.446970799 | 0.048 | 0.011 | 1.26E−09 |
| GRCh38-------RSPO3 | 4.88E−14 | 3.418633852 | 0.03 | 0.003 | 1.43E−09 |
| Rattus-norvegicus-C1qb | 5.98E−14 | −2.214631273 | 0.009 | 0.041 | 1.75E−09 |
| GRCh38-------EPHA4 | 9.61E−14 | 2.946565408 | 0.036 | 0.006 | 2.81E−09 |
| GRCh38-------PLAT | 1.48E−13 | −3.760990935 | 0.001 | 0.024 | 4.33E−09 |
| Rattus-norvegicus-Igfbp6 | 1.56E−13 | −2.411780086 | 0.006 | 0.035 | 4.57E−09 |
| GRCh38-------DOK4 | 6.60E−13 | 2.522190044 | 0.035 | 0.006 | 1.93E−08 |
| GRCh38-------SCN7A | 8.63E−13 | −3.685702807 | 0.001 | 0.023 | 2.52E−08 |
| GRCh38-------EPHA7 | 5.83E−12 | 3.887186861 | 0.023 | 0.001 | 1.70E−07 |
| GRCh38-------COLEC11 | 1.01E−11 | −5.557827985 | 0 | 0.017 | 2.95E−07 |
| GRCh38-------CACNA2D3 | 1.01E−11 | −5.592593403 | 0 | 0.017 | 2.95E−07 |
| GRCh38-------NSG1 | 1.01E−11 | −5.592593403 | 0 | 0.017 | 2.95E−07 |
| GRCh38-------CPXM1 | 1.20E−11 | −2.740844362 | 0.004 | 0.026 | 3.50E−07 |
| GRCh38-------ATP1B4 | 1.36E−11 | 5.581083733 | 0.019 | 0 | 3.99E−07 |
| GRCh38-------EFNA5 | 1.75E−11 | −2.169902331 | 0.008 | 0.034 | 5.12E−07 |
| GRCh38-------ANGPT1 | 1.83E−11 | −5.557827985 | 0 | 0.017 | 5.35E−07 |
| GRCh38-------ACSM3 | 1.83E−11 | −5.692129077 | 0 | 0.017 | 5.35E−07 |
| GRCh38-------HCN4 | 2.88E−11 | 2.462689032 | 0.032 | 0.006 | 8.43E−07 |
| GRCh38-------SESN3 | 3.46E−11 | −2.659707599 | 0.004 | 0.026 | 1.01E−06 |
| GRCh38-------SPARCL1 | 6.02E−11 | −5.522204075 | 0 | 0.016 | 1.76E−06 |
| Rattus-norvegicus- | 6.58E−11 | −2.809085223 | 0.003 | 0.024 | 1.92E−06 |
| NEWGENE-2134 | | | | | |
| Rattus-norvegicus-Islr | 6.80E−11 | −2.163750104 | 0.007 | 0.032 | 1.99E−06 |
| GRCh38-------SYT1 | 8.74E−11 | −4.785238481 | 0 | 0.017 | 2.55E−06 |
| GRCh38-------SULF2 | 9.36E−11 | −3.550773227 | 0.001 | 0.02 | 2.74E−06 |
| GRCh38-------GRIA4 | 1.98E−10 | −5.485678199 | 0 | 0.015 | 5.80E−06 |
| GRCh38-------TBX3 | 2.07E−10 | 5.354312871 | 0.017 | 0 | 6.06E−06 |
| GRCh38-------B4GALNT3 | 4.85E−10 | 2.30751866 | 0.029 | 0.006 | 1.42E−05 |
| GRCh38-------KCNQ5 | 5.01E−10 | 2.692808816 | 0.026 | 0.004 | 1.47E−05 |
| GRCh38-------NRK | 6.43E−10 | −2.84413217 | 0.003 | 0.021 | 1.88E−05 |
| GRCh38-------MMP16 | 6.87E−10 | −2.180098098 | 0.005 | 0.026 | 2.01E−05 |
| GRCh38-------GBP4 | 6.87E−10 | −3.937241574 | 0.001 | 0.016 | 2.01E−05 |
| GRCh38-------MYO15B | 9.68E−10 | −3.30461264 | 0.001 | 0.018 | 2.83E−05 |
| GRCh38-------PIEZO2 | 2.79E−09 | −2.382479312 | 0.004 | 0.023 | 8.16E−05 |
| Rattus-norvegicus-Col11a1 | 2.84E−09 | −2.20027598 | 0.006 | 0.026 | 8.32E−05 |
| GRCh38-------ENO2 | 4.79E−09 | 2.875012147 | 0.021 | 0.003 | 0.000140095 |
| Rattus-norvegicus-Dbp | 4.80E−09 | −2.154472291 | 0.006 | 0.025 | 0.00014044 |
| Rattus-norvegicus-Spon1 | 4.82E−09 | −2.307191184 | 0.004 | 0.022 | 0.000140887 |
| GRCh38-------ARHGAP28 | 5.51E−09 | −3.337779504 | 0.001 | 0.017 | 0.000161121 |
| GRCh38-------CALN1 | 5.76E−09 | −2.396673193 | 0.003 | 0.021 | 0.000168359 |
| GRCh38-------UPP1 | 8.31E−09 | 3.738323475 | 0.016 | 0.001 | 0.000242931 |
| Rattus-norvegicus-Wfdc1 | 1.08E−08 | −2.692129077 | 0.003 | 0.019 | 0.000315385 |
| GRCh38-------COL6A2 | 1.16E−08 | −2.007630902 | 0.006 | 0.025 | 0.000338196 |
| GRCh38-------ASPN | 1.24E−08 | −2.254723764 | 0.004 | 0.022 | 0.00036327 |
| GRCh38-------PTGIS | 1.29E−08 | −5.107166576 | 0 | 0.012 | 0.000378119 |
| GRCh38-------PDGFRB | 2.35E−08 | −5.058256975 | 0 | 0.011 | 0.00068754 |
| GRCh38-------TNFRSF12A | 2.74E−08 | 2.7472566 | 0.019 | 0.003 | 0.000801293 |
| GRCh38-------AC112487.1 | 3.15E−08 | −3.126275399 | 0.001 | 0.015 | 0.000921844 |
| GRCh38-------RALYL | 3.27E−08 | 4.323285976 | 0.014 | 0 | 0.000956293 |
| GRCh38-------ISLR | 3.29E−08 | −2.692129077 | 0.003 | 0.018 | 0.000961686 |
| GRCh38-------PPFIA2 | 3.30E−08 | −2.692129077 | 0.003 | 0.018 | 0.000963839 |
| GRCh38-------SHOX2 | 3.37E−08 | −2.819185813 | 0.002 | 0.017 | 0.000984757 |
| GRCh38-------MIR503HG | 4.28E−08 | −4.955163482 | 0 | 0.011 | 0.001250629 |
| GRCh38-------PLPP3 | 4.28E−08 | −4.955163482 | 0 | 0.011 | 0.001250629 |
| GRCh38-------SLC22A17 | 4.28E−08 | −4.955163482 | 0 | 0.011 | 0.001250629 |
| GRCh38-------LGI2 | 5.77E−08 | −2.592593403 | 0.003 | 0.017 | 0.001687113 |
| GRCh38-------TBX18 | 7.78E−08 | −4.900715698 | 0 | 0.011 | 0.002275896 |
| GRCh38-------CCDC3 | 7.78E−08 | −4.955163482 | 0 | 0.011 | 0.002275928 |
| Rattus-norvegicus-Cfb | 8.68E−08 | −2.370200982 | 0.003 | 0.019 | 0.002537126 |
| Rattus-norvegicus-Ccl5 | 9.43E−08 | −2.489782598 | 0.003 | 0.018 | 0.002756759 |
| GRCh38-------ITGB8 | 1.11E−07 | −4.058256975 | 0 | 0.011 | 0.003255865 |
| GRCh38-------LIPA | 1.42E−07 | −4.955163482 | 0 | 0.01 | 0.004143723 |
| GRCh38-------KDR | 2.42E−07 | 2.414433864 | 0.017 | 0.003 | 0.007069871 |
| GRCh38-------AC092691.1 | 2.58E−07 | −4.84413217 | 0 | 0.01 | 0.007548432 |
| GRCh38-------TMEM176A | 2.58E−07 | −4.84413217 | 0 | 0.01 | 0.007548432 |
| GRCh38-------CSGALNACT1 | 2.58E−07 | −4.900715698 | 0 | 0.01 | 0.007548513 |
| GRCh38-------SPOCK2 | 2.58E−07 | −4.900715698 | 0 | 0.01 | 0.007548513 |
| GRCh38-------SLC24A3 | 2.58E−07 | −4.955163482 | 0 | 0.01 | 0.007548586 |
| GRCh38-------NRP2 | 2.98E−07 | −2.832544196 | 0.001 | 0.013 | 0.008719152 |
| GRCh38-------IFI16 | 3.85E−07 | −2.232697458 | 0.004 | 0.018 | 0.011259714 |
| GRCh38-------SCUBE3 | 4.34E−07 | 2.414433864 | 0.018 | 0.003 | 0.012678401 |
| Rattus-norvegicus-Rarres1 | 4.62E−07 | −3.422668402 | 0.001 | 0.011 | 0.013500649 |
| GRCh38-------CDON | 5.28E−07 | −3.154472291 | 0.001 | 0.012 | 0.015444552 |
| GRCh38-------PLAC9 | 5.60E−07 | −2.56284606 | 0.002 | 0.015 | 0.016374049 |
| GRCh38-------IL33 | 5.66E−07 | −2.370200982 | 0.002 | 0.015 | 0.016560018 |
| GRCh38-------TMEM163 | 5.67E−07 | 3.092505769 | 0.015 | 0.002 | 0.016576378 |
| GRCh38-------RAB3C | 6.69E−07 | −3.955163482 | 0 | 0.01 | 0.019545176 |
| GRCh38-------BANCR | 7.55E−07 | 3.38468652 | 0.013 | 0.001 | 0.022081301 |
| Rattus-norvegicus-Cib1 | 8.34E−07 | −3.370200982 | 0.001 | 0.011 | 0.024391529 |
| GRCh38-------MAN1C1 | 9.59E−07 | 2.192041442 | 0.02 | 0.005 | 0.028025838 |
| GRCh38-------SEMA3D | 9.85E−07 | −2.409729346 | 0.002 | 0.014 | 0.028793199 |
| GRCh38-------VCAM1 | 1.01E−06 | 2.38468652 | 0.017 | 0.003 | 0.029518032 |
| GRCh38-------AC007563.2 | 1.19E−06 | 3.30222436 | 0.012 | 0.001 | 0.034686147 |
| GRCh38-------ADM | 1.64E−06 | 2.969649021 | 0.013 | 0.001 | 0.048069552 |
| GRCh38-------ELMO1 | 1.69E−06 | −3.2446701 | 0.001 | 0.011 | 0.049410446 |
| GRCh38-------KCNK6 | 1.70E−06 | −3.007630902 | 0.001 | 0.011 | 0.049622514 |
| GRCh38-------CDH10 | 1.85E−06 | 4.969649021 | 0.01 | 0 | 0.054182258 |
| GRCh38-------SLC27A6 | 2.15E−06 | 2.79972402 | 0.014 | 0.002 | 0.06285421 |
| GRCh38-------BIRC5 | 3.12E−06 | 2.418633852 | 0.015 | 0.003 | 0.091171008 |
| Rattus-norvegicus-C1qtnf5 | 3.14E−06 | −2.062772457 | 0.004 | 0.017 | 0.091910542 |
| Rattus-norvegicus-Tra2a | 4.18E−06 | −2.239804345 | 0.003 | 0.015 | 0.122071765 |
| GRCh38-------IQGAP3 | 4.59E−06 | 3.214761519 | 0.011 | 0.001 | 0.134106973 |
| GRCh38-------MTUS2 | 4.70E−06 | 2.032384776 | 0.023 | 0.007 | 0.137533802 |
| GRCh38-------ROR2 | 4.88E−06 | 2.006174897 | 0.02 | 0.005 | 0.142552075 |
| GRCh38-------PLXDC1 | 4.92E−06 | −3.259169669 | 0.001 | 0.01 | 0.143701099 |
| GRCh38-------GBP2 | 5.15E−06 | −2.392921058 | 0.002 | 0.013 | 0.150553254 |
| GRCh38-------CCSER1 | 5.39E−06 | 2.354312871 | 0.016 | 0.003 | 0.157650243 |
| GRCh38-------TLCD4 | 6.32E−06 | 2.240296611 | 0.014 | 0.002 | 0.184751934 |
| GRCh38-------TRABD2B | 9.52E−06 | 2.829471363 | 0.013 | 0.002 | 0.27824139 |
| GRCh38-------PAPPA | 9.57E−06 | −2.522204075 | 0.002 | 0.012 | 0.279647924 |
| GRCh38-------NYNRIN | 9.71E−06 | −2.84413217 | 0.001 | 0.01 | 0.283991166 |
| GRCh38-------DLGAP1 | 9.77E−06 | −2.659707599 | 0.001 | 0.01 | 0.28561077 |
| GRCh38-------HLA-B | 1.21E−05 | −2.159633996 | 0.003 | 0.014 | 0.352897075 |
| GRCh38-------EFNB2 | 1.38E−05 | −2.2446701 | 0.003 | 0.013 | 0.403098313 |
| GRCh38-------PHGDH | 1.38E−05 | −2.20027598 | 0.003 | 0.013 | 0.403922101 |
| GRCh38-------XIRP2 | 1.48E−05 | 2.314297193 | 0.013 | 0.002 | 0.434042909 |
| GRCh38-------RASL12 | 1.67E−05 | −2.473294475 | 0.002 | 0.011 | 0.488418435 |
| GRCh38-------PLEKHA2 | 1.72E−05 | 2.047651533 | 0.014 | 0.003 | 0.501822883 |
| GRCh38-------HEY1 | 1.73E−05 | −2.84413217 | 0.001 | 0.01 | 0.50668767 |
| GRCh38-------HDAC2-AS2 | 1.77E−05 | 3.168957829 | 0.01 | 0.001 | 0.518643738 |
| GRCh38-------MT-ATP8 | 1.78E−05 | 3.022116441 | 0.01 | 0.001 | 0.519322278 |
| GRCh38-------C12orf75 | 1.89E−05 | 2.157276024 | 0.014 | 0.003 | 0.551893251 |
| GRCh38-------ATP8A2 | 2.66E−05 | −2.250902053 | 0.002 | 0.012 | 0.777722612 |
| GRCh38-------DCLK1 | 2.91E−05 | −2.473294475 | 0.002 | 0.011 | 0.849949643 |
| GRCh38-------AL591519.1 | 3.45E−05 | 2.500163738 | 0.012 | 0.002 | 1 |
| GRCh38-------FDPS | 3.45E−05 | 2.462689032 | 0.012 | 0.002 | 1 |
| GRCh38-------BRCA2 | 3.69E−05 | 2.657705015 | 0.01 | 0.001 | 1 |
| GRCh38-------DEPDC1 | 4.30E−05 | 2.085126239 | 0.014 | 0.003 | 1 |
| GRCh38-------PCDH11X | 4.50E−05 | 2.870113348 | 0.011 | 0.002 | 1 |
| GRCh38-------CKS1B | 4.54E−05 | 2.477795925 | 0.011 | 0.002 | 1 |
| Rattus-norvegicus-RT1-Bb | 4.58E−05 | −2.20027598 | 0.002 | 0.011 | 1 |
| Rattus-norvegicus- | 4.59E−05 | 2.178235643 | 0.014 | 0.003 | 1 |
| AABR07044900.1 | | | | | |
| GRCh38-------EMILIN1 | 4.85E−05 | −2.007630902 | 0.003 | 0.014 | 1 |
| GRCh38-------CRACD | 5.06E−05 | −2.422668402 | 0.002 | 0.011 | 1 |
| Rattus-norvegicus-Ccn3 | 5.39E−05 | −2.785238481 | 0.001 | 0.01 | 1 |
| GRCh38-------CD44 | 5.42E−05 | −2.463310386 | 0.001 | 0.01 | 1 |
| GRCh38-------MED12L | 5.73E−05 | 2.657705015 | 0.01 | 0.001 | 1 |
| Rattus-norvegicus-Sacs | 5.73E−05 | 2.657705015 | 0.01 | 0.001 | 1 |
| GRCh38-------FEN1 | 5.74E−05 | 2.607078942 | 0.01 | 0.001 | 1 |
| GRCh38-------MT-ND4L | 7.01E−05 | 2.38468652 | 0.011 | 0.002 | 1 |
| Rattus-norvegicus- | 7.82E−05 | −2.250902053 | 0.002 | 0.011 | 1 |
| MGC108823 | | | | | |
| GRCh38-------CKAP2L | 8.13E−05 | 2.214761519 | 0.011 | 0.002 | 1 |
| GRCh38-------STYXL2 | 9.05E−05 | 2.079831939 | 0.012 | 0.003 | 1 |
| Rattus-norvegicus-Anks1b | 0.000165472 | 2.725723438 | 0.01 | 0.002 | 1 |
| GRCh38-------MPP7 | 0.000166036 | 2.38468652 | 0.01 | 0.002 | 1 |
| Rattus-norvegicus-Ptcd3 | 0.000175659 | 2.18438787 | 0.019 | 0.007 | 1 |
| Rattus-norvegicus-Pygm | 0.000189253 | 2.121652115 | 0.011 | 0.002 | 1 |
| GRCh38-------PDGFD | 0.000218824 | −2.087801251 | 0.003 | 0.012 | 1 |
| Rattus-norvegicus-Per3 | 0.000228107 | −2.14780856 | 0.002 | 0.01 | 1 |
| Rattus-norvegicus-Fmr1 | 0.000228495 | −2.036777248 | 0.002 | 0.01 | 1 |
| GRCh38-------ETV5 | 0.0002877 | 2.121652115 | 0.01 | 0.002 | 1 |
| Rattus-norvegicus-Obscn | 0.000335041 | 2.012717743 | 0.013 | 0.004 | 1 |
| GRCh38-------ENC1 | 0.000387131 | −2.036777248 | 0.002 | 0.01 | 1 |
| Rattus-norvegicus-Cdh13 | 0.000484067 | 2.047651533 | 0.011 | 0.003 | 1 |
| GRCh38-------COL2A1 | 0.000663069 | 2.022116441 | 0.01 | 0.002 | 1 |
| GRCh38-------TMEM223 | 0.000663069 | 2.022116441 | 0.01 | 0.002 | 1 |
| Rattus-norvegicus-Hba-a2 | 0.000730405 | −2.401909841 | 0.003 | 0.011 | 1 |

Day 21 CM alone vs CM + RADA16

| | | | | | |
|---|---|---|---|---|---|
| Rattus-norvegicus-Hba-a2 | 2.63E−226 | −4.30861 | 0.006 | 0.069 | 8.49E−222 |
| Rattus-norvegicus-Hbb-b1 | 2.42E−112 | −4.44404 | 0.002 | 0.033 | 7.82E−108 |
| Rattus-norvegicus-AC112568.1 | 1.25E−84 | −2.7967 | 0.007 | 0.035 | 4.04E−80 |
| GRCh38-------GRK5 | 2.36E−72 | −2.2001 | 0.008 | 0.036 | 7.63E−68 |
| GRCh38-------COX7A1 | 1.66E−71 | −3.76595 | 0.002 | 0.021 | 5.37E−67 |
| GRCh38-------DES | 8.47E−47 | −2.12176 | 0.005 | 0.022 | 2.73E−42 |
| GRCh38-------NPPB | 5.97E−44 | −3.21223 | 0.002 | 0.015 | 1.93E−39 |
| Rattus-norvegicus-Comp | 3.03E−38 | 2.137704 | 0.016 | 0.003 | 9.79E−34 |
| GRCh38-------AC107068.2 | 5.55E−36 | −2.15456 | 0.005 | 0.018 | 1.79E−31 |
| Rattus-norvegicus-Cxcl9 | 4.62E−28 | −2.89319 | 0.001 | 0.01 | 1.49E−23 |
| GRCh38-------DNAJA4 | 5.08E−26 | −2.2747 | 0.002 | 0.011 | 1.64E−21 |

TABLE S2
Sarcomere orientation
Summary of statistical measures of deviation in sarcomere orientation for
two treatments, CM alone and CM + RADA16, observed over 7 days and 3
months periods. The table includes the mean, median, standard deviation,
minimum, and maximum values for each treatment and observation period.

| Observation | | | | “Standard | | |
|---|---|---|---|---|---|---|
| Period | Treatment | Mean | Median | Deviation” | Min | Max |
| 7 days | CM alone | −4.47E−15 | −3.18 | 58.8 | −76.7 | 107 |
| | CM + RADA16 | −5.43E−15 | −22.9 | 56.7 | −65.4 | 109 |
| 3 months | CM alone | 7.85E−15 | −29 | 50.5 | −53.5 | 128 |
| | CM + RADA16 | 5.18E−15 | 4.84 | 40.6 | −90 | 100 |

TABLE S3
Primer Information

| Primers for qPCR | Source | Sequence |
|---|---|---|
| MYH6 forward | | GCCCTTTGACATTCGCACTG (SEQ ID NO: 1) |
| MYH6 reverse | | GGTTTCAGCAATGACCTTGCC (SEQ ID NO: 2) |
| MYH7 forward | | TCACCAACAACCCCTACGATT (SEQ ID NO: 3) |
| MYH7 reverse | | CTCCTCAGCGTCATCAATGGA (SEQ ID NO: 4) |
| TNNI1 forward | | CCGGAAGTCGAGAGAAAACCC (SEQ ID NO: 5) |
| TNNI1 reverse | | TCAATGTCGTATCGCTCCTCA (SEQ ID NO: 6) |
| TNNI3 forward | | TTTGACCTTCGAGGCAAGTTT (SEQ ID NO: 7) |
| TNNI3 reverse | | CCCGGTTTTCCTTCTCGGTG (SEQ ID NO: 8) |
| GJA1 forward | | GGTGACTGGAGCGCCTTAG (SEQ ID NO: 9) |
| GJA1 reverse | | GCGCACATGAGAGATTGGGA (SEQ ID NO: 10) |
| HCN4 forward | | TGGACACCGCTATCAAAGTGG (SEQ ID NO: 11) |
| HCN4 reverse | | CTGCCGAACATCCTTAGGGA (SEQ ID NO: 12) |
| KCNJ2 forward | | TGGATGCTGGTTATCTTCTGC (SEQ ID NO: 13) |
| KCNJ2 reverse | | AGCCTATGGTTGTCTGGGTCT (SEQ ID NO: 14) |
| NANOG forward | | GGACACTGGCTGAATCCTTCC (SEQ ID NO: 15) |
| NANOG reverse | | CTCGCTGATTAGGCTCCAACC (SEQ ID NO: 16) |
| GAPDH forward | | GGAGCGAGATCCCTCCAAAAT (SEQ ID NO: 17) |
| GAPDH reverse | | GGCTGTTGTCATACTTCTCATGG (SEQ ID NO: 18) |

TABLE S4
Full sequencing metadata

| | | | | Total reads mapped |
|---|---|---|---|---|
| | | | | against genome and |
| | Harvest | | | matched to array |
| Puck ID | Timepoint | Treatment | Total reads given | barcodes |
| Puck_220928_03 | Day 7 | CM alone | 386,386,538 | 90,011,603 |
| Puck_220928_09 | Day 7 | CM + RADA16 | 535,123,383 | 139,639,136 |
| Puck_220928_10 | Day 7 | CM + RADA16 | 393,997,777 | 92,640,743 |
| Puck_220928_12 | Day 7 | CM alone | 322,085,384 | 78,098,542 |
| Puck_220928_13 | Day 7 | CM alone | 284,889,901 | 65,705,917 |
| Puck_220928_19 | Day 21 | CM + RADA16 | 333,557,537 | 68,559,235 |
| Puck_220928_22 | Day 7 | CM + RADA16 | 450,089,418 | 54,250,698 |
| Puck_220928_23 | Day 7 | CM + RADA16 | 365,677,061 | 64,233,734 |
| Puck_220928_27 | Day 21 | CM + RADA16 | 371,175,139 | 66,106,173 |
| Puck_220928_29 | Day 21 | CM alone | 378,744,696 | 79,139,131 |
| Puck_220928_30 | Day 21 | CM alone | 368,331,722 | 109,614,778 |
| Puck_220928_32 | Day 21 | CM alone | 389,020,615 | 89,114,737 |
| Puck_220928_33 | Day 21 | CM + RADA16 | 412,285,993 | 79,417,588 |
| Puck_220928_34 | Day 21 | CM + RADA16 | 295,931,459 | 89,423,876 |
| Puck_220928_38 | Day 21 | CM alone | 355,736,984 | 82,377,841 |
| Puck_220928_39 | Day 21 | CM alone | 478,758,027 | 74,574,826 |

1. A method of maturing a population of cardiomyocytes derived from human-induced pluripotent stem cells (hiPSC-CMs) comprising contacting the one or more hiPSC-CMs with a self-assembling peptide (SAP).

2. The method of claim 1, wherein the SAP is selected from the group consisting of RADA16, IEIK13, KLD12, and QLEL12.

3. (canceled)

4. The method of claim 1, wherein the population of hiPSC-CMs contacted with the SAP express one or more of MYH7/6, TNNI3/1, GJA1, MYL2 and KCNJ2.

5.-8. (canceled)

9. The method of claim 1, wherein the population of hiPSC-CMs contacted with the SAP exhibit decreased expression of HCN4 as compared to control hiPSC-CMs.

10. The method of claim 1, wherein automaticity of the hiPSC-CMs is reduced after contact with the SAP.

11.-16. (canceled)

17. The method of claim 1, wherein the population of hiPSC-CMs and the SAP are co-administered to a subject via syringe or catheter.

18. (canceled)

19. (canceled)

20. The method of claim 17, wherein the SAP promotes engraftment and/or vascularization of the population of hiPSC-CMs in the subject, or
wherein the SAP promotes sarcomere organization of the population of hiPSC-CMs in the subject,
wherein the population of hiPSC-CMs contacted with the SAP exhibited increased sarcomere length upon administration to the subject, as compared to a population of hiPSC-CMs alone,
wherein the SAP promotes improved electrophysiological integration of the hiPSC-CMs upon administration to the subject, optionally wherein the electrophysiological integration of the hiPSC-CMs is monitored via mesh nanoelectronics.

21.-50. (canceled)

51. A pharmaceutical composition comprising a population of stem cell-derived cells or precursors thereof and a self-assembling peptide (SAP).

52. The pharmaceutical composition of claim 51, wherein the SAP is selected from the group consisting of RADA16, IEIK13, KLD12, and QLEL12.

53. The pharmaceutical composition of claim 51, wherein the SAP comprises RADA16.

54. The pharmaceutical composition of claim 51, further comprising a pharmaceutically acceptable carrier or excipient.

55. The pharmaceutical composition of claim 51, further comprising RBI-PVbBB.

56. A method of maturing a stem cell-derived cell or a precursor thereof comprising contacting the stem cell-derived cell or precursor thereof with at least one self-assembling peptide (SAP).

57. The method of claim 56, wherein the stem cell-derived cell is selected from the group consisting of beta cells, alpha cells, delta cells, enterochromaffin cells, endothelial cells, satellite cells, cardiomyocytes, dermal cells, hematopoietic cells, and precursors thereof.

58. The method of claim 56, wherein the self-assembling peptide is selected from the group consisting of RADA16, IEIK13, KLD12, and QLEL12.

59. The method of claim 56, wherein the stem cell-derived cell is contacted with the SAP upon co-administration of the stem cell-derived cell and the SAP to a subject.

60. The method of claim 56, wherein the stem cell-derived cell is contacted with the SAP in a suspension prior to administration to a subject.

61. The method of claim 59, wherein the subject is a mammal.

62. The method of claim 59, wherein the stem cell-derived cell and the SAP are co-administered to the subject via catheter or syringe.

63. The pharmaceutical composition of claim 51, wherein the population of stem cell-derived cells is selected from the group consisting of beta cells, alpha cells, delta cells, enterochromaffin cells, endothelial cells, satellite cells, cardiomyocytes, dermal cells, hematopoietic cells, and precursors thereof.


SEQUENCE LISTING (18 sequences)

SEQ ID NO: 1 - DNA, 20 bp, Synthetic construct
  source 1..20: mol_type = other DNA
gccctttgacattcgcactg

SEQ ID NO: 2 - DNA, 21 bp, Synthetic construct
  source 1..21: mol_type = other DNA
ggtttcagcaatgaccttgcc

SEQ ID NO: 3 - DNA, 21 bp, Synthetic construct
  source 1..21: mol_type = other DNA
tcaccaacaacccctacgatt

SEQ ID NO: 4 - DNA, 21 bp, Synthetic construct
  source 1..21: mol_type = other DNA
ctcctcagcgtcatcaatgga

SEQ ID NO: 5 - DNA, 21 bp, Synthetic construct
  source 1..21: mol_type = other DNA
ccggaagtcgagagaaaaccc

SEQ ID NO: 6 - DNA, 21 bp, Synthetic construct
  source 1..21: mol_type = other DNA
tcaatgtcgtatcgctcctca

SEQ ID NO: 7 - DNA, 21 bp, Synthetic construct
  source 1..21: mol_type = other DNA
tttgaccttcgaggcaagttt

SEQ ID NO: 8 - DNA, 20 bp, Synthetic construct
  source 1..20: mol_type = other DNA
cccggttttccttctcggtg

SEQ ID NO: 9 - DNA, 19 bp, Synthetic construct
  source 1..19: mol_type = other DNA
ggtgactggagcgccttag

SEQ ID NO: 10 - DNA, 20 bp, Synthetic construct
  source 1..20: mol_type = other DNA
gcgcacatgagagattggga

SEQ ID NO: 11 - DNA, 21 bp, Synthetic construct
  source 1..21: mol_type = other DNA
tggacaccgctatcaaagtgg

SEQ ID NO: 12 - DNA, 20 bp, Synthetic construct
  source 1..20: mol_type = other DNA
ctgccgaacatccttaggga

SEQ ID NO: 13 - DNA, 21 bp, Synthetic construct
  source 1..21: mol_type = other DNA
tggatgctggttatcttctgc

SEQ ID NO: 14 - DNA, 21 bp, Synthetic construct
  source 1..21: mol_type = other DNA
agcctatggttgtctgggtct

SEQ ID NO: 15 - DNA, 21 bp, Synthetic construct
  source 1..21: mol_type = other DNA
ggacactggctgaatccttcc

SEQ ID NO: 16 - DNA, 21 bp, Synthetic construct
  source 1..21: mol_type = other DNA
ctcgctgattaggctccaacc

SEQ ID NO: 17 - DNA, 21 bp, Synthetic construct
  source 1..21: mol_type = other DNA
ggagcgagatccctccaaaat

SEQ ID NO: 18 - DNA, 23 bp, Synthetic construct
  source 1..23: mol_type = other DNA
ggctgttgtcatacttctcatgg